(19) United States
(12) Patent Application Publication
(10) Pub. No.: US 2024/0006080 A1
(43) Pub. Date: Jan. 4, 2024

(54) TECHNIQUES FOR GENERATING PREDICTIVE OUTCOMES RELATING TO ONCOLOGICAL LINES OF THERAPY USING ARTIFICIAL INTELLIGENCE

(71) Applicant: Hoffmann-La Roche Inc., Little Falls, NJ (US)

(72) Inventors: Silvia Elena MOLERO LEON, La Aurora, Heredia (CR); Turap Tasoglu, Basel (CH)

(73) Assignee: Hoffmann-La Roche Inc., Little Falls, NJ (US)

(21) Appl. No.: 18/039,346

(22) PCT Filed: Oct. 6, 2021
(86) PCT No.: PCT/US2021/053764

§ 371 Date: May 30, 2023

(30) Foreign Application Priority Data
Dec. 7, 2020 (EP) 20212280.0

(51) Int. Cl. G16H 50/70; G16H 10/60; G16B 40/20

(52) U.S. Cl. CPC G16H 50/70; G16H 10/60; G16B 40/20

(57) ABSTRACT

Disclosed are techniques for using artificial intelligence (AI) to facilitate the selection of lines of therapy for subjects diagnosed with cancer. Methods and systems disclosed herein relate to techniques for using AI to predict therapeutic outcomes and cancer evolution in subjects based on mutation profiles of subjects across cancer types, to predict treatment survival prospects for subjects using enriched subject-specific data sets, and to automatically validate whether the reasons (e.g., represented by features in a subject record) that contributed to the selection of a particular line of therapy comply with oncological treatment guidelines.

### CROSS-REFERENCE TO RELATED APPLICATION

This application claims the benefit of and priority to European Patent Application No. 20212280.0, filed on Dec. 7, 2020, which is hereby incorporated by reference in its entirety for all purposes.

### FIELD

Methods and systems disclosed herein generally relate to techniques for using artificial intelligence (AI) to facilitate the selection of a line of therapy for a subject diagnosed with cancer. More specifically, methods and systems disclosed herein relate to techniques for using AI to: (1) predict therapeutic outcomes and cancer evolution in a subject based on mutational profiles of other subjects across cancer types; (2) predict subject-specific side effects of a candidate line of therapy for treating cancer; and/or (3) automatically validate whether the reasons (e.g., represented by certain features in a subject record) that contributed to the selection of a particular line of therapy comply with oncological treatment guidelines.

### BACKGROUND

Cancer is one of the leading causes of death globally. Cancers can develop at any location within the human body. There are, however, several common locations where cancer can develop. For example, leading cancer types include cancers of the breast, lung, colon, and blood. Regardless of the type, cancer involves the unconstrained division of some of the body's cells, which can potentially spread to other tissue around the body. In healthy individuals, cell divisions that create new cells are generally balanced with the death of older or damaged cells. In individuals diagnosed with cancer, however, this balance breaks down. Cancer causes the uncontrolled growth of abnormal cells in the body, even when new cells are not needed. The unrestricted growth of the abnormal cells can form a tumor in tissue of the body. In some cases, the abnormal cells can break away from the tumor, travel through the body's bloodstreams, and attach to tissue in new areas of the body to potentially form new tumors.

The uncontrolled growth of these abnormal cells is caused by genetic mutations in cellular deoxyribonucleic acid (DNA). Genetic mutations are often caused by inherited genetics. However, mutations can also be triggered by environmental factors. For example, toxic exposure (e.g., exposure to carcinogens, radiation, and tobacco), lifestyle-related factors (e.g., obesity, diet, and alcohol consumption), age, medications, hormones, random chance, and certain infections (e.g., hepatitis, human papilloma virus (HPV), and Epstein-Barr virus) can cause cancer-related genomic mutations in an otherwise healthy individual.

Oncology, which is the study and treatment of cancerous cells, presents several unique and significant challenges. First, certain cancers can be caused by a complex combination of multiple mutations across different genes. Modern cancer research suggests that the evolution of a cancer pathway in a subject involves complex dependencies and interactions between multiple genetic mutations. A cancer often develops when the protein produced by one mutation interacts with the protein produced by another mutation. For example, in certain blood cancers, subjects fare far worse when the primary mutation JAK2 V617F (the driving mutation) is activated before a secondary mutation, identified as TET2. Conversely, subjects who had the TET2 mutation activate before the JAK2 V617F driving mutation had much better clinical outcomes. Moreover, due to advances in genomic testing, a subject's specific molecular subsets can be identified and evaluated for selecting specific treatments, given their molecular characteristics. However, with these advances, many challenges have arisen, such as obtaining the correct genotyping of tumor samples. Thus, identifying lines of therapy for treating cancers is uniquely challenging over other diseases because targeting a primary mutation with, for example, genetic replacement therapy can activate or exacerbate the impact of a secondary mutation, which can make the cancer worse. Isolating causes of cancer can, therefore, be significantly challenging.

Second, oncological lines of therapy often involve levels of toxicity that can be harmful to subjects. For example, depending on subject-specific risk factors, certain chemotherapies and immunosuppressants can create a life-threatening side effect in the subject. The treatment selection for cancer is, therefore, heavily dependent on an individual's unique progression-free survival. Further, there is a wide and diverse spectrum of side effects in response to lines of therapy. Additionally, treatment selection varies depending on the subject's subjective risk tolerance. For example, if a group of subjects with the same cancer at the same stage has a three-year survival probability of 15%, subjects in the group would be willing to accept different aggressiveness of treatment, and a portion of the group may be willing to accept aggressive treatment, such as high-dose radiotherapy, whereas a different portion of the group may only be willing to accept less-aggressive treatment, such as combination therapy. Therefore, treatment selection and side-effect assessments are uniquely challenging in the oncological context.

Third, certain lines of therapy require authorization before being performed. For example, a physician seeking to perform a gene replacement therapy on a subject may need prior authorization if the therapy targets a different mutation than the mutation that is commonly targeted by other therapies. Associations such as the National Comprehensive Cancer Network (NCCN) and the American Society of Clinical Oncology (ASCO) have established guidelines for treating cancer. Identifying whether the reasons underlying the selection of lines of therapy for a subject comply with existing guidelines is difficult because it is challenging to identify the features that contributed to treatment selection. In some cases, a literature review may be needed. As treatments are often selected using the treating physician's knowledge base, objectively identifying the features that contribute to the selection of a treatment is difficult.

US 2020/0370124 discloses systems and methods for predicting the efficacy of a cancer therapy in a subject. The systems and methods disclosed are predicated on the determination that the number, percentage, or ratio of particular types of single nucleotide variations (SNVs) in the nucleic acid of a subject with cancer who responds to therapy is different to that of a subject who does not respond to therapy. SNVs identified in a nucleic acid molecule can be used to determine a plurality of metrics forming a profile whereupon subjects that are likely to respond to cancer therapy typically have a different profile to subjects that are unlikely to respond to cancer therapy. The plurality of metrics are then applied to a computational model where the computational model selected based on specific subject attributes. The computational model determines a therapy indicator, for example, a numerical percentage, based on the plurality of metrics where the therapy indicator is indicative of a predicted responsiveness to cancer therapy.

Thus, there is a need to improve personalized selection of lines of therapy for subjects diagnosed with cancer, personalized assessments of side effects, and verification that lines of therapy comply with existing guidelines, so as to improve treatment efficacy for individual subjects diagnosed with cancer.

### SUMMARY

In some embodiments, a computer-implemented method is provided for predicting subject-specific outcomes of oncological lines of therapy. The method can include identifying a particular subject having been diagnosed with a type of cancer and retrieving a genomic data set corresponding to the particular subject. A line of therapy can be proposed to be performed on the particular subject. The genomic data set can include a mutational profile, which can include the molecular characteristics of a subject's tumor, such the molecular pattern, a mutation order (e.g., indicating a series of multiple genetic mutations that mutated at different times), and so on. The computer-implemented method can also include identifying a set of other subjects having been diagnosed with the same type of cancer as the subject. Each other subject may have undergone the line of therapy and may be associated with a treatment outcome. The computer-implemented method can also include retrieving another genomic data set for each other subject of the set of other subjects. The other genomic data set can include another mutation profile. The computer-implemented method can include inputting, for each other subject of the set of other subjects, the mutational profile of the particular subject and the other mutational profile of the other subject into a trained similarity model. The trained similarity model may have been trained to generate a similarity weight representing a predicted degree to which the mutational profile of the particular subject is similar to the other mutational profile of the other subject. The computer-implemented method can include determining, based on the similarity weights outputted by the trained similarity model, a predicted treatment outcome of performing the line of therapy on the particular subject. Upon determining that at least one of the similarity weights outputted by the similarity model is within a threshold, the computer-implemented method can include identifying one of the other subjects based on the determination and assigning the treatment outcome of the identified other subject as the predicted treatment outcome for the particular subject. Upon determining that none of the similarity weights outputted by the similarity model is within the threshold, then the computer-implemented method can include identifying another set of subjects having been diagnosed with a different type of cancer than the particular subject to search for a mutational profile that is similar to the mutational profile of the particular subject.

In some embodiments, a system is provided that includes one or more data processors and a non-transitory, computer-readable storage medium containing instructions which, when executed on the one or more data processors, cause the one or more data processors to perform part or all of one or more methods disclosed herein.

In some embodiments, a computer-program product is provided that is tangibly embodied in a non-transitory, machine-readable storage medium and that includes instructions configured to cause one or more processors to perform part or all of one or more methods disclosed herein.

Some embodiments of the present disclosure include a system including one or more processors. In some embodiments, the system includes a non-transitory, computer-readable storage medium containing instructions which, when executed on the one or more processors, cause the one or more processors to perform part or all of one or more methods and/or part or all of one or more processes disclosed herein. Some embodiments of the present disclosure include a computer-program product tangibly embodied in a non-transitory, machine-readable storage medium, including instructions configured to cause one or more processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present disclosure is described in conjunction with the appended figures:

FIG. 1 illustrates a network environment in which the cloud-based application is hosted, according to some aspects of the present disclosure.

FIG. 2 is a flowchart illustrating an example of a process performed by the cloud-based application to distribute condensed subject records to user devices in association with a consult broadcast requesting assistance with treating a subject, according to some aspects of the present disclosure.

FIG. 3 is a flowchart illustrating an example of a process for monitoring the user integration of treatment-plan definitions (e.g., decision trees or treatment workflows) and automatically updating the treatment-plan definitions based on a result of the monitoring, according to some aspects of the present disclosure.

FIG. 4 is a flowchart illustrating an example of a process for recommending treatments for a subject, according to some aspects of the present disclosure.

FIG. 5 is a flowchart illustrating an example of a process for obfuscating query results to comply with data-privacy rules, according to some aspects of the present disclosure.

FIG. 6 is a flowchart illustrating an example of a process for communicating with users using bot scripts, such as a chatbot, according to some aspects of the present disclosure.

FIG. 7 is a block diagram illustrating an example of a network environment for deploying trained AI models to facilitate the subject-specific identification of treatments and treatment schedules for subjects diagnosed with cancer, according to some aspects of the present disclosure.

FIG. 8 is a block diagram illustrating an example of a network environment for deploying a trained AI model to predict the treatment outcomes and cancer evolution for subjects diagnosed with cancer, according to some aspects of the present disclosure.

FIG. 9 is a block diagram illustrating an example of a network environment for deploying a trained AI model to predict the subject-specific side effects of oncological lines of therapy, according to some aspects of the present disclosure.

FIG. 10 is a block diagram illustrating an example of a network environment for deploying a trained AI model to identify the factors that contribute to the selection of a given line of therapy, according to some aspects of the present disclosure.

FIG. 11 is a flowchart illustrating an example of a process for predicting the treatment outcomes and cancer evolution for subjects diagnosed with cancer, according to some aspects of the present disclosure.

FIG. 12 is a flowchart illustrating an example of a process for predicting the subject-specific side effects of mutation-targeting treatments, according to some aspects of the present disclosure.

FIG. 13 is a flowchart illustrating an example of a process for deploying AI models to identify the factors that contribute to the selection of a given treatment, according to some aspects of the present disclosure.

In the appended figures, similar components and/or features can have the same reference label. Further, various components of the same type can be distinguished by a dash following the reference label and by a second label that distinguishes among the similar components. If only the first reference label is used in the specification, the description is applicable to any one of the similar components having the same first reference label irrespective of the second reference label.

### DETAILED DESCRIPTION

### I. Overview

Cancer is an incredibly complex disease. It can develop anywhere in the human body. In some cases, cancer is hereditary, while in other cases, cancer can develop in response to environmental factors. Regardless of the origin of cancer's development, there is often a complex combination of genetic mutations along the evolution of cancer pathways. For instance, a tumor consists of billions of cells, and different mutations can exist in each cell individually. Monitoring and responding to the evolution of cancer is, therefore, an extremely challenging task because cancerous cells can evolve or adapt to lines of therapy.

In the oncological context, understanding the underlying mechanisms of cancer typically involves frequently obtaining genomic data of cancerous cells to detect changes in the cancerous cells. Modern oncological practices use genomic data to identify the specific genetic mutations that are contributing to the cancerous cell growth and the order of the genetic mutations. The mutational profile can include molecular characteristics of a tumor, such as the order in which individual genetic mutations activate (e.g., mutation order). In certain cases, cancer can develop after a specific group of gene mutations have activated according to a pattern indicated by a mutational profile. Therefore, using genomic data to facilitate the identification of mutations is beneficial. However, identifying the appropriate lines of therapy to treat the cancerous cells has another complicated web of considerations. Additionally, identifying oncological lines of therapy is particularly challenging due to the wide range of side effects exhibited across subjects diagnosed with cancer and the uncertainty of treatment outcomes.

Certain aspects of the present disclosure relate to deploying AI models trained to perform tasks that solve complex cancer-specific problems. AI techniques can yield predictive outcomes from dense or seemingly unconnected data sets to assist physicians with clinical decision making when treating subjects diagnosed with cancer. Certain aspects of the present disclosure provide a cloud-based oncology application configured with an AI system that can perform predictive functionality. AI-based techniques can be used to learn patterns and correlations across complex data sets of various data types (e.g., structured data sets, unstructured data sets, streaming data) from disparate sources. Even though oncological diseases are characterized by complexity and uncertainty, certain aspects of the present disclosure relate to executing specialized AI models to facilitate the selection of lines of therapy in a manner that is contextual to the genomic profile of an individual subject.

Certain aspects of the present disclosure relate to an AI system configured to perform certain predictive functionality, such as predicting therapeutic outcomes and subsequent cancer evolution for an individual subject (e.g., a patient) based on mutational profile of subjects across cancer types, predicting subject-specific side effects in response to lines of therapy, and automatically verifying whether the reasons for selecting a line of therapy (e.g., a specific target therapy for treating breast cancer) for an individual subject to comply with oncological guidelines.

Certain aspects of the present disclosure relate to a cloud-based oncology application configured to generate a prediction of a therapeutic outcome of a line of therapy proposed to be performed on an individual subject. The prediction can be based on the mutational profile of subjects having the same cancer or having a different cancer type as the individual subject. For example, a mutational profile represents, among other molecular characteristics, the order in which genes mutate over time (e.g., the mutation order or a pattern of mutations). The mutational profile can impact clinical decisions relating to diagnostics and selecting lines of therapy. Certain aspects of the present disclosure relate to executing specialized similarity-based AI models that have been trained to automatically identify, for example, when the mutational profile of a subject with breast cancer is similar to the mutational profile of another subject with lung cancer. For instance, the target therapy performed on the subject with lung cancer can be informative regarding the efficacy of certain lines of therapy for the subject with breast cancer. The specialized similarity-based AI models can be trained based on a training data set of pairs or mutational profiles (one mutational profile representing one subject and the other mutational profile representing another subject) of subjects with the same or different type of cancer. Each pair can be labeled as being similar or not similar. Learning algorithms can be executed to automatically learn which patterns indicated by mutational profiles are similar to each other. Once trained, the specialized similarity-based AI models can output a similarity weight, which is a value representing a degree to which one mutational profile of a subject is similar to another mutational profile of another subject.

Certain aspects of the present disclosure also relate to a cloud-based oncology application configured to generate a prediction of side effects of a line of therapy based on the context of the characteristics of a particular subject. The oncology application can be used to build a graphical mapping between lines of therapy and the various side effects associated with the lines of therapy. In some examples, the graphical mapping may represent an ontology, which describes the types of therapeutic lines, the properties of each therapeutic line (e.g., the side effects, the progression-free survival), and the relationship between the therapeutic lines and the properties. The graphical mapping can be stored as a knowledge graph, which is accessed each time a user requests a subject-specific prediction of the side effects of a line of therapy. When a user operates the oncology application to request a prediction of the subject-specific side effects of a line of therapy, the oncology application can query the knowledge graph using the subject features of the particular subject. A reasoning engine can perform a logical inference task that identifies which treatments and/or side effects in the knowledge graph are logically related to the subject features of the particular subject. The output of the reasoning engine represents the subject-specific side effects of the line of therapy. It will be appreciated that the present disclosure is not limited to mapping lines of therapy to their corresponding side effects. The progression-free survival of the lines of therapy or any other variables can be graphically mapped and stored as an ontology in a knowledge graph.

Certain aspects of the present disclosure also relate to a cloud-based oncology application configured to evaluate subject data of cancer subjects with certain cancer types and the treatments performed on those cancer subjects to automatically learn, using AI-based algorithms, the reasons why the treatment was assigned to each individual cancer subject. For example, the oncology application can automatically predict that the reason why certain lung cancer subjects are treated with a specific target therapy treatment is that those lung cancer subjects have a driver mutation in the HER2 gene. The oncology application can then compare the predicted reasons for various treatments against a set of guidelines or rules established by authoritative medical associations, such as the NCCN and the ASCO. Where no guidelines exist, the oncology application can also identify candidates for new guidelines based on the treatments performed to target specific mutations, the corresponding therapeutic outcomes of those treatments, and the progression-free survival of subjects after the treatments were performed.

An application (e.g., operating locally on a device and/or at least partly using results of computations performed at one or more remote and/or cloud servers) can be used by (for example) a subject who has cancer and/or a care provider caring for a subject that has cancer. The application can perform one or more operations disclosed herein. In some instances, one or more applications can facilitate communicate between a subject with cancer and a care provider. While the oncology application relates to oncology-specific treatment workflows, in some implementations, the application can relate to other specific cancer types, such as a cloud-based breast cancer application, a cloud-based lung cancer application, a cloud-based colon cancer application, a cloud-based hematological cancer application, and so on. Each application specific to a cancer type can be distinct from other applications, for example, based on the variables that the applications make available. Such communication may (for example) facilitate alerting a care provider of an abnormal symptom and/or may facilitate telemedicine (e.g., which may be particularly valuable when the subject or a portion of a local society has a communicable disease, when the subject has a locomotion disability, and/or when the subject is physically far from an office of the care provider).

### II. Summary of Cancer Sub-Types, Diagnosis Protocol, Pertinent Medical Tests, Progression Assessment, and Available Treatments

IIA. Cause of Cancer

According to the World Health Organization, about one in six deaths can be attributed to cancer, making it the second leading cause of death globally. Cancer is a group of diseases characterized by uncontrolled growth of abnormal cells in the body. This uncontrolled growth is caused by genetic changes, such as mutations, in cellular DNA. Although these mutations are often caused by inherited genetics or disposition, other factors, including environmental/toxic exposure (e.g., exposure to carcinogens, radiation, and tobacco), lifestyle-related factors (e.g., obesity, diet, and alcohol consumption), age, medications, hormones, random chance, and infections (e.g., hepatitis, HPV, and Epstein-Barr virus) can cause cancer-related genomic changes in an individual. Although progress has been made in screening, diagnosis, and treatment, cancer rates are increasing as more people live longer and engage in causative lifestyle behaviors.

II.B. Types of Cancer

There are more than one hundred types of cancer, including cancers that form solid tumors, such as breast, skin, lung, colon, and prostate cancer, to name a few. According to the American Institute for Cancer Research, there were an estimated 18 million cancer cases around the world in 2018. Of these, 9.5 million cases were men and 8.5 million were women. Lung and breast cancers were the most common cancers worldwide, each contributing to about 12.3% of the total number of new cases in 2018. Lung cancer was the most common cancer in men while breast cancer was the most common cancer in women worldwide. Colorectal cancer was the third most common cancer, with 1.8 million new cases in 2018, followed by prostate cancer as the fourth most common cancer, with more than 1.275 million new cases in 2018.

Cancers also include blood or hematological cancers which affect the production and function of blood cells. Examples include leukemias (e.g., acute leukemia, acute lymphocytic leukemia, acute myelocytic leukemias, and chronic lymphocytic leukemia (CLL)), lymphomas (e.g., Hodgkin's disease or non-Hodgkin's disease lymphomas (e.g., diffuse anaplastic lymphoma kinase (ALK) negative, large B-cell lymphoma (DLBCL); follicular lymphoma (FL); diffuse ALK positive DLBCL; ALK positive, ALK+anaplastic large-cell lymphoma (ALCL); acute myeloid lymphoma (AML)); and multiple myeloma.

II.B.1. Breast Cancer

Breast cancer is the most common invasive cancer in women, but it can also occur in men. Breast cancer often develops in cells from the lining of the milk ducts and the lobules that supply these ducts with milk. Cancers developing from the ducts are known as ductal carcinomas, while those developing from lobules are known as lobular carcinomas. Although rare, inflammatory breast cancer is another type of breast cancer that accounts for about 1-5% of all breast cancers. These cancers can be broadly divided into sub-groups depending on certain biomarkers that have been established to predict response to treatment: (1) hormone receptor (ER+ and/or PR+) positive and Her2 negative (Her2-breast cancer, (2) hormone receptor positive (ER+ and/or PR+) and Her2 positive (Her2+) breast cancer, (3) hormone receptor negative (ER−) and Her2 positive (Her2+) breast cancer, and (4) hormone receptor negative (ER−) and Her2 negative (Her2−) (triple negative) breast cancer.

II.B.1.i. Clinical Symptoms

Symptoms of breast cancer include a lump in the breast, bloody discharge from the nipple, thickening or swelling of the breast, breast pain, irritation or dimpling of breast skin, redness or flaky skin in the nipple or breast, nipple pain, itchiness, change in breast color, or a rash on the breast.

II.B.1.ii. Diagnosis

Although numerous clinical symptoms are associated with breast cancer, breast cancer is often identified through routine mammography screening. Breast cancer can be diagnosed through multiple tests, including a mammogram, ultrasound, magnetic resonance imaging (MRI), and a biopsy.

Genetic testing for mutations (for example, BRCA1 and BRCA2 mutations) associated with increased risk of breast cancer can also be performed after breast cancer is diagnosed, to determine the best course of treatment. Other diagnostic assays (for example, the VENTANA Her2Dual ISH test (Roche, Basel, Switzerland)) can be used to identify HER2 positive breast cancers for targeted therapy with trastuzumab (Herceptin, Roche, Basel, Switzerland).

There are generally four stages of breast cancer, characterized by the medical community as follows:

Stage 0 is the earliest stage of breast cancer. At this stage, there are abnormal cells present, but the cancer has not spread to other parts of the breast. This stage is often referred to a carcinoma in situ or non-invasive.

Stage 1 is the earliest stage of invasive breast cancer, meaning the cancer has grown or spread into nearby or surrounding breast tissue. The tumor is usually about 2 centimeters in size, or smaller. At this stage the cancer may or may not have spread into the lymph nodes.

Stage 2 is also indicative of invasive breast cancer, and at this stage the tumor may have grown to about 5 centimeters, and sometimes larger. The cancer may or may not have spread into the lymph nodes.

Stage 3 is a stage of invasive breast cancer where the cancer has usually spread to the lymph nodes. Inflammatory breast cancers start at Stage 3 since they involve the skin.

Stage 4 is often referred to as “metastatic” and means the cancer has spread beyond the breast and nearby lymph nodes to other parts of the body.

II.B.1.iii. Subtyping

Once breast cancer has been diagnosed, to determine the course of treatment, the breast cancer is often subtyped based on the hormone receptors expressed by the tumor cells. The four main female breast cancer subtypes, are as follows, in order of prevalence:

(1) hormone receptor (ER+ and/or PR+) positive and Her2 negative (Her2-breast cancer (luminal A breast cancer), (2) hormone receptor negative (ER−) and Her2 negative (Her2−) (triple negative) breast cancer, (3) hormone receptor positive (ER+ and/or PR+) and Her2 positive (Her2+) breast cancer (luminal B breast cancer), and (4) hormone receptor negative (ER−) and Her2 positive (Her2+) breast cancer (HER2-enriched breast cancer).

II.B.1.iv. Treatment

The standard of care for breast cancer is a multidisciplinary approach incorporating surgery, radiotherapy, and drug treatment. Standard of care for breast cancer is determined by both disease (e.g., tumor, stage, pace of disease) and patient characteristics (e.g., age, by biomarker expression and intrinsic phenotype). General guidance on treatment options is described in the NCCN Guidelines (e.g., NCCN Clinical Practice Guidelines in Oncology, Breast Cancer, version 2.2016, National Comprehensive Cancer Network, 2016, pp. 1-202), and in the ESMO Guidelines (e.g., Senkus, E., et al. Primary Breast Cancer: ESMO Clinical Practice Guidelines for diagnosis, treatment and follow-up. Annals of Oncology 2015; 26(Suppl. 5): v8-v30; and Cardoso F., et al. Locally recurrent or metastatic breast cancer: ESMO Clinical Practice Guidelines for diagnosis, treatment and follow-up. Annals of Oncology 2012; 23 (Suppl. 7):vii11-vii19.).

II.B.1.iv.a. Early or Non-Metastatic Breast Cancer

The standard of care for early or non-metastatic breast cancer is typically a mastectomy or breast-conserving surgery, followed by radiation therapy or systemic therapy.

If the subject is hormone receptor (ER+ and/or PR+) positive and Her2 negative (Her2−), endocrine therapy (e.g., tamoxifen, GnRH agonists, aromatase inhibitors), with or without chemotherapy, can be administered. When chemotherapy is administered, its type and dosage are selected depending on tumor burden and/or biomarker expression. Neoadjuvant therapy to reduce tumor burden prior to surgery can also be used. Exemplary neoadjuvant therapies include tamoxifen or an aromatase inhibitor, with or without chemotherapy.

If the subject is hormone receptor (ER+ and/or PR+) positive and Her2 positive (Her2+), hormone therapy and anti-Her2 therapy, with or without chemotherapy, can be administered. Exemplary treatments include administration of trastuzumab (Herceptin® (Roche, Basel, Switzerland)), chemotherapy and tamoxifen, or an aromatase inhibitor. Neoadjuvant therapy (e.g., administration of trastuzumab or pertuzamab, with chemotherapy) can also be used.

If the subject is hormone receptor negative (ER−) and Her2 positive (Her2+), anti-Her2 therapy and chemotherapy can be administered. Neoadjuvant therapy (for example, administration of trastuzumab or pertuzamab, with chemotherapy) can also be used.

If the subject is hormone receptor negative (ER−) and Her2 negative (Her2−), chemotherapy can be administered. Chemotherapy can also be administered as neoadjuvant therapy.

Numerous chemotherapeutic agents are available for the treatment of early or non-metastatic breast cancer, including, but not limited to, cyclophosphamide (Cytoxan), docetaxel (Taxotere), paclitaxel (Taxol), doxorubicin (Adriamycin), epirubicin (Ellence), and methotrexate (Maxtrex), which can be administered as single therapies or combination therapies. For example, for the treatment of Her2+ breast cancers, docetaxel, carboplatin, and trastuzumab can be administered in combination. Other examples include administration of trastuzumab and paclitaxel, or administration of doxorubicin and cyclophosphamide followed by administration of paclitaxel and trastuzumab.

II.B.1.iv.b. Advanced or Metastatic Breast Cancer

The standard of care for advanced or metastatic breast cancer is often surgery. In some instances, chemotherapy is administered before or after surgery. Radiation therapy and/or hormone therapy (for tumors that are ER+ positive) can be administered after surgery.

If the subject is hormone receptor (ER+ and/or PR+) positive and postmenopausal, hormone therapy can include tamoxifen, an aromatase inhibitor (anstrozole, letrozole, or exemestane), a cyclin-dependent kinase inhibitor (palbociclib), or fluvestrant (anti-estrogen therapy).

If the subject is hormone receptor (ER+ and/or PR+) positive and premenopausal, hormone therapy can include tamoxifen or an LHRH agonist. Targeted therapy such as trastuzumab (Herceptin (Roche, Basel, Switzerland)), bevacizumab (Avastin® (Roche, Basel, Switzerland)), lapatinib, pertuzumab, mTOR inhibitors, T-DM1 (trastuzumab emtansine), or palbociclib and letrozole can also be administered. In some instances, if the subject is Her2+, then (1) pertuzamab alone, (2) trastuzumab and pertuzumab, (3) trastuzumab and chemotherapy, or (4) lapatinib and chemotherapy are administered to the subject as first-line therapies. In some cases, Avastin® is administered in combination with paclitaxel to treat HER2-negative breast cancer in patients who have not yet received chemotherapy for metastatic breast cancer.

Numerous chemotherapeutic agents are available for the treatment of advanced or metastatic breast cancer including, but not limited to, capecitabine (Xeloda® (Roche, Basel, Switzerland)), gemcitabine (Cynzar), carboplatin (Paraplatin), cisplatin (Platinol), cyclophosphamide (C) (Cytoxen), docetaxel (T) (Taxotere), paclitaxel, (T) (Taxol), doxorubicin (A) (Adriamycin), epirubicin (E) (Ellence), eribulin (Halaven), 5-fluorouracil (5-FU, Adrucil), Ixabepilone (Ixempra), liposomal doxorubicin (doxil), methotrexate (M) (Maxtrex), albumin bound paclitaxel (Abraxane), and vinorelbine (Navelbine).

II.B.1.iv.c. Early or Non-Metastatic Breast Cancer

Standard of care for triple negative breast cancer (TNBC), is determined by both disease (stage, pace of disease, etc.) and patient (age, co-morbidities, symptoms, etc.) characteristics.

Patients with early and potentially resectable locally advanced TNBC (i.e., without distant metastatic disease) are managed with locoregional therapy (surgical resection with or without radiation therapy), with or without systemic chemotherapy.

Surgical treatment can be breast conserving (i.e., a lumpectomy, which focuses on removing the primary tumor with a margin), or can be more extensive (i.e., mastectomy, which aims for complete removal of all of the breast tissue). Radiation therapy is typically administered post-surgery to the breast/chest wall and/or regional lymph nodes, with the goal of killing microscopic cancer cells left post-surgery. In the case of breast-conserving surgery, radiation is administered to the remaining breast tissue and sometimes to the regional lymph nodes (including axillary lymph nodes). In the case of a mastectomy, radiation may still be administered if factors that predict higher risk of local recurrence are present.

Depending on tumor and patient characteristics, chemotherapy may be administered in the adjuvant (post-operative) or neoadjuvant (pre-operative) setting. Additional guidance for treating early and locally advanced TNBC is provided in Solin L J., Clin Br Cancer. 2009, 9:96-100; Freedman G M, et al. Cancer. 2009, 115:946-951; Heemskerk-Gerritsen B A M, et al. Ann Surg Oncol. 2007, 14:3335-3344; and Kell M R, et al. MBJ. 2007, 334:437-438.

Systemic chemotherapy is the standard treatment for patients with metastatic TNBC, although no standard regimen or sequence exists and options for cytotoxic chemotherapy are the same as those for other subtypes. Single-agent cytotoxic chemotherapeutic agents such as anthracyclines (e.g., doxorubucin, epirubicine), taxanes (e.g., paclitaxel, docetaxel), anti-metabolites (e.g., capecitabine, gemcitabine), non-taxane microtubule inhibitors (e.g., vinorelbine, eribulin, exabepilone), platinum (e.g., cisplatin, carboplatin), and aklylating agents (e.g., cyclophosphamide) are generally regarded as the primary option for patients with metastatic TNBC, although combination chemotherapy regimens may be used when there is aggressive disease and visceral involvement. Treatment may also involve sequential rounds of different single-agent treatments. Palliative surgery and radiation may be utilized, as appropriate, to manage local complications.

II.B.2. Colorectal Cancer

Colorectal cancer, also known as bowel cancer or colon cancer, is any cancer that affects the colon and/or rectum. Colorectal cancer begins in the large intestine (colon). Although colon cancer typically affects older adults, it can happen at any age. It usually begins as small, noncancerous clumps of cells, called polyps, that form on the inside of the colon. Over time, some of these polyps can become colon cancers.

II.B.2.i. Clinical Symptoms

Symptoms of colon cancer include rectal bleeding or blood in stool, cramps, gas, abdominal pain, a persistent change in bowel habits, including diarrhea or constipation, weakness or fatigue, and unexplained weight loss. Many people with colon cancer experience no symptoms in the early stages of the disease. When symptoms appear, they will likely vary depending on the cancer's size and location in the large intestine.

II.B.2.ii. Diagnosis

Physicians recommend screening tests for healthy subjects, with no signs or symptoms of colon cancer, to look for signs of colon cancer or noncancerous colon polyps. Doctors generally recommend that people with an average risk of colon cancer begin screening around age 50. Finding colon cancer at its earliest stage provides the greatest chance for successful treatment.

In addition to a physical examination, one or more of the following tests may be used to diagnose colorectal cancer: colonoscopy, biopsy, molecular testing of a tumor, blood test, computed tomography (CT or CAT) scan, MRI, proctoscopy, ultrasound, and X-ray. In many cases, if a suspected colorectal cancer is found by any screening or diagnostic test, it is biopsied during a colonoscopy.

When a biopsy indicates the presence of colon cancer, additional genetic tests may be performed to further classify the colon cancer. For example, changes in any of the mismatch repair genes (MLH1, MSH2, MSH6, and PMS2) can be detected to identify subjects with Lynch syndrome, a hereditary disorder that increases a person's risk of developing colon cancer.

The stages of colon cancer have been characterized by the medical community as follows:

Stage 0 is the earliest stage of colon cancer. This stage is also known as carcinoma in situ or intramucosal carcinoma (Tis). At this stage, the cancer has not grown beyond the inner layer (mucosa) of the colon or rectum.

Stage I is characterized by cancer growth through the muscularis mucosa into the submucosa, and it may also have grown into the muscularis propria. It has not spread to nearby lymph nodes or to distant sites.

Stage IIA is characterized by cancer growth into the outermost layers of the colon or rectum but has not gone through them. At this stage, the cancer has not spread to nearby lymph nodes or to distant sites. Stage II colon cancer can be subdivided into three stages:
Stage IIA-Cancer has spread to the serosa or outer colon wall, but not beyond that outer barrier.
Stage IIB-Cancer has spread past the serosa but has not affected nearby organs.
Stage IIC-Cancer has affected the serosa and the nearby organs.

Stage III is characterized by cancer growth past the lining of the colon that has affected the lymph nodes. In this stage, even though the lymph nodes are affected, the cancer has not yet affected other organs in the body. This stage is further divided into three categories: IIIA-IIIC. Where the cancer is staged in these categories depends on a complex combination of which layers of the colon wall are affected and how many lymph nodes have been attacked.

Stage IV is characterized by metastatic growth that has spread to other organs in the body through the blood and lymph nodes.

II.B.2.iii. Treatment

The standard of care for colon cancer depends on the stage of colon cancer. Stages 0-III colon cancers are typically treated with surgery.

Treatment for Stage 0 colon cancer is usually a polypectomy, performed during a colonoscopy. During this procedure, a physician may remove all of the malignant cells. If the cells have affected a larger area, an excision may be performed during the colonoscopy.

For Stage I colon cancer patients, a partial colectomy is performed to remove the affected area. This surgical procedure may involve rejoining the parts of the colon that are still healthy.

Stage II cancers are treated with surgery to remove the affected areas. Chemotherapy may also be recommended in some cases. High-grade or abnormal cancer cells or tumors that have caused a blockage or perforation of the colon may warrant further treatment. If the surgeon is unable to remove all of the cancer cells, radiation may also be recommended to kill any remaining cancer cells and reduce the risk of a recurrence.

All categories of Stage III colon cancer involve surgery to remove the affected areas. Optionally, chemotherapy and/or radiation therapy can be administered. In some instances, radiation therapy may also be recommended for patients who are not healthy enough for surgery or for patients who may still have cancer cells in their bodies after surgery has taken place.

Patients with Stage IV colon cancer may undergo surgery to remove small areas, or metastases, in the organs that have been affected. In many cases, however, the areas are too large to be removed. Therefore, targeted therapies, usually in combination with chemotherapy, are used to treat Stage IV/metastatic cancers (mCRC).

Although there is no single standard of care for mCRC, common first-line treatment regimens include administration of a fluoropyrimidine (e.g., fluorouracil (5-FU) or capecitabine) in various combinations and schedules with irinotecan and/or oxaliplatin. Bevacizumab (Avastin®) cetuximab or panitumumab may be combined with any of the first-line chemotherapy treatments, for example, with Xeloda. In some cases, maintenance therapy is administered. Administration of maintenance therapy will depend on the selection of first-line chemotherapy, but is often a combination of a fluoropyrimidine and bevacizumab.

Second-line therapies can also be used. Further to the treatments listed above, depending on first-line therapy choice, aflibercept or ramucirumab can be used in combination with FOLFIRI (fluorouracil+leucovorin+irinotecan).

Third-line therapies can also be used. For example, if the cancer is RAS wild-type and has not been previously treated with EGFR antibodies, cetuximab or panitumumab can be administered, optionally, in combination with chemotherapy. Regorafenib, or a combination of trifluridine and tipiracil, can be also be used as third-line therapies. In some cases, colorectal patients who are not likely to respond to anti-EGFR monoclonal antibody therapies can be identified using the Cobas® KRAS Mutation Test or Cobas® KRAS Mutation Test v2 (Roche, Basel, Switzerland), which detects mutations in codons 12, 13, and 61 in the KRAS gene, in formalin-fixed, paraffin-embedded tissue, from colorectal cancer patients.

II.B.3. Lung Cancer

Lung cancer typically starts in the cells lining the bronchi and parts of the lung, such as the bronchioles or alveoli. About 80-85% of lung cancers are non-small-cell lung cancer (NSCLC), which can be divided into the following subtypes: adenocarcinoma, squamous cell carcinoma, and large-cell carcinoma. These subtypes are often grouped together as NSCLC because their treatment and prognoses are often similar. About 10-15% of all lung cancers are small-cell lung carcinoma (SCLC), which tends to grow and spread faster than NSCLC.

II.B.3.i. Clinical Symptoms

Symptoms of lung cancer include a persistent cough, coughing up blood, chest pain, hoarseness, loss of appetite, unexplained weight loss, shortness of breath, fatigue, infections that do not resolve, and wheezing.

II.B.3.ii. Diagnosis

Lung cancer can be detected using imaging tests (e.g., an X-ray, CT scan, or MRI), sputum cytology, and/or a tissue biopsy. Biopsies can be performed using bronchoscopy, mediastinoscopy, or a needle biopsy. A biopsy sample can also be obtained from lymph nodes or from tissues where the cancer may have spread from, for example, the liver.

Once a lung cancer diagnosis is made, the type and stage of the lung cancer are determined. Staging tests may include imaging procedures that allow a physician to determine whether the cancer has spread beyond the lungs. These tests include CT, MRI, positron emission tomography (PET), and bone scans.

Several diagnostic assays for stratification and typing of lung cancer are available. For example, the VENTANA ROS1 (SP384) Rabbit Monoclonal Primary Antibody assay (Roche, Basel, Switzerland) is available for identification of ROS-1 positive cancer, an aggressive form of cancer, that occurs in about 1-2% of NSCLC patients. The VENT AN A ALK (D5F3) CDx assay (Roche, Basel, Switzerland) is available as an aid in identifying NSCLC patients eligible for treatment with XALKORI® (crizotinib), ZYKADIA® (ceritinib), or ALECENSA® (alectinib). The p40 (BC28) Mouse Monoclonal Primary Antibody assay (Roche, Basel, Switzerland), the TTF-1 (SP141) Rabbit Monoclonal Primary Antibody assay (Roche, Basel, Switzerland), the Cytokeratin 5/6 (D5/16B4) Mouse Monoclonal Primary Antibody assay (Roche, Basel, Switzerland), and the Napsin A (MRQ-60) Mouse Monoclonal Primary Antibody assay (Roche, Basel, Switzerland) can also be used to stratify lung cancers.

II.B.3.ii.a. NSCLC

The stages of NSCLC are as follows:

Stage 0 is also known as carcinoma in situ. At this stage, the cancer is small in size and has not spread into deeper lung tissue or outside the lungs.

Stage I is characterized by cancer that is in a single lung, which may be present in the underlying lung tissue but has not spread to the lymph nodes. This stage is divided into Stages Ia and Ib. In Stage Ia, the tumor is 3 centimeters or smaller. In Stage Ib, the tumor is between 3 and centimeters in size, or the tumor is 4 centimeters or smaller and one or more of the following is found: (1) cancer has spread to the main bronchus but has not spread to the carina; (2) cancer has spread to the innermost layer of the membrane that covers the lung; and/or (3) part of the lung or the whole lung has collapsed or has developed pneumonitis.

Stage II involves possible spread to the nearby lymph nodes and into the chest wall. This stage is divided into Stages IIa and IIb. A Stage IIa cancer describes a tumor larger than 4 centimeters but 5 centimeters or less in size that has not spread to the nearby lymph nodes. A Stage IIb lung cancer describes a tumor that is 5 centimeters or less in size that has spread to the lymph nodes. A Stage IIb cancer can also be a tumor more than five centimeters wide that has not spread to the lymph nodes.

Stage III involves continued spread from the lungs to the lymph nodes. If the cancer has spread only to lymph nodes on the same side of the chest where the cancer started, it is called Stage IIIa. If the cancer has spread to the lymph nodes on the opposite side of the chest, or above the collar bone, it is called Stage IIIb.

Stage IV is the most advanced, metastatic stage of the disease. At this stage, the cancer has metastasized beyond the lungs into other areas of the body. About 40% of NSCLC patients are diagnosed when they are in Stage IV, with a five-year survival rate of less than 10%.

II.B.3.ii.b. SCLC

The stages of SCLC have been characterized by the medical community as follows:

The limited stage or Stage 1 of SCLC is a lung cancer that has only developed on one side of the chest and involves a single area of the lung, lymph nodes, or both.

The extensive stage or Stage 2 of SCLC is a lung cancer that has spread to the opposite side of the chest, outside the chest, or to other parts of the body.

II.B.3.iii. Treatment

II.B.3.iii.a. NSCLC

Surgery is often recommended for patients with Stage I or II NSCLC and may provide the best possibility for a cure. Surgery (or radiation if the patient is not a surgical candidate), with or without adjuvant chemotherapy, based on risk factors is generally appropriate for Stages Ib and II.

Standard of care for NSCLC Stage I and II is surgery with adjuvant chemotherapy. For example, a platinum chemotherapeutic, such as cisplatin or carboplatin, can be administered in combination with vinorelbine, etoposide, vinblastine, gemcitabine, docetaxel, pemetrexed, or paclitaxel.

Standard of care for locally advanced disease (Stage IIIa or IIIb) is chemoradiation therapy. Treatment recommendations include the use of concurrent chemotherapy and radiation, or sequential chemotherapy and radiation. Selected patients (predominantly those with Stage IIIa) may be surgical candidates; these patients may receive chemotherapy alone or chemotherapy with radiation before surgical resection. Stage IIIa and IIb disease are typically treated with a combination of chemotherapy and radiation if the patient is not a surgical candidate.

Chemotherapy and radiation therapy are preferably given concurrently, but in patients with poor performance status, these therapies may be given sequentially. The decision to treat the patient with concurrent chemoradiation rather than surgery, radiation, or chemotherapy individually should be made by a multidisciplinary team that includes a medical oncologist, a radiation therapist, and a thoracic surgeon.

Patients with metastatic disease (Stage IV) or recurrent disease after primary therapy (e.g., surgery and/or radiation) should be considered for first-line chemotherapy in order to improve quality of life, palliate symptoms, and improve overall survival. For example, a platinum chemotherapeutic, such as cisplatin or carboplatin, can be administered in combination with vinorelbine, etoposide, vinblastine, gemcitabine, docetaxel, pemetrexed, or paclitaxel.

Single-agent therapy with, for example, paclitaxel, docetaxel, gemcitabine, vinorelbine, or pemetrexeb is a reasonable first-line option in patents with good performance status or in the elderly.

Second-line chemotherapy can be administered for metastatic or recurrent disease after disease progression following first-line therapy. Exemplary second-line regimens are as follows: nivolumab; pembrolizumab in tumors that are PD-L1 positive (patients with EGFR or ALK genomic tumor aberrations should have disease progression prior to receiving pembrolizumab); docetaxel and ramucirumab; nintedanib and docetaxel; erlotinib (Tarceva® (Roche, Basel, Switzerland)); and afatinib. Erlotinib alone, in second-line settings, remains the standard of care.

Third-line chemotherapy is given for advanced or recurrent NSCLC, after disease progression following first-line and second-line therapy. Options include erlotinib, ramucirumab, and nivolumab.

Maintenance chemotherapy for metastatic or recurrent disease, in the form of switch maintenance chemotherapy or continuation maintenance therapy, may be considered for patients with advanced (Stage IV) disease who have a disease response or stable disease after completing first-line chemotherapy.

Switch maintenance chemotherapy involves administering chemotherapy with agents that are different from those used in first-line therapy. Continuation maintenance therapy involves giving chemotherapy that includes an agent that was part of the first-line therapy, after completion of four to six cycles of first-line therapy.

II.B.3.iii.b. SCLC

SCLC of any stage is typically initially responsive to treatment, but responses are usually short-lived. Chemotherapy, with or without radiation therapy, is given depending on the stage of disease. In many patients, chemotherapy prolongs survival and improves quality of life enough to warrant its use. Surgery generally plays no role in treatment of SCLC, although it may be curative in the rare patient who has a small focal tumor without spread (such as a solitary pulmonary nodule) and who underwent surgical resection before the tumor was identified as SCLC.

Limited-stage SCLC is generally treated with combinations of chemotherapy drugs. For example, a platinum chemotherapeutic, such as cisplatin or carboplatin, can be administered in combination with vinorelbine, etoposide, vinblastine, gemcitabine, docetaxel, pemetrexed, or paclitaxel.

For extensive-stage SCLC, chemotherapy alone, either as single agent therapy or combination therapy, is often used. Irinotecan, topotecan, vinca alkaloids (e.g., vinblastine, vincristine, vinorelbine), alkylating agents (e.g., cyclophosphamide, ifosfamide), doxorubicin, taxanes (e.g., docetaxel, paclitaxel), and gemcitabine are examples of such chemotherapeutic agents. Some combinations include a platinum chemotherapeutic, such as cisplatin or carboplatin, in combination with etoposide, irinotecan, topotecan, and gemcitabine. In some instances, cyclophosphamide, doxorubicin, and vincristine are administered as first-line chemotherapy.

Patients who have relapsed disease more than six months after completing first-line chemotherapy can be treated with the original first-line regimen (typically a platinum-based combination) again.

II.B.4. Hematologic Cancer

Most hematologic or blood cancers start in the bone marrow, where blood cells are made. Blood cancers occur when abnormal blood cells grow out of control and interrupt the function of normal blood cells. There are three primary types of blood cancers, as set forth below.

Leukemias occur when the body creates too many abnormal white blood cells and interferes with the bone marrow's ability to make red blood cells and platelets.

Lymphomas are blood cancers that affect the lymphatic system. In lymphomas, abnormal, mutated lymphocytes grow of control and produce more abnormal lymphocytes. Over time, these abnormal lymphocytes become lymphoma cells which damage the immune system.

Myelomas are cancers of the plasma cells. Plasma cells are white blood cells that produce disease- and infection-fighting antibodies. Myeloma cells prevent the normal production of antibodies, thus leaving the body's immune system weakened and susceptible to infection.

II.B.4.i. Clinical Symptoms

Symptoms of blood cancer include anemia, poor blood clotting, unusual bruising, bleeding gums, rash, heavy periods, bowel movements that are black or streaked with red, fever, night sweats, lumps in the neck or armpit, unexplained weight loss, and bone pain.

II.B.4.ii. Diagnosis

For diagnosing leukemia, a physical exam and a complete blood count (CBC) test, which can identify abnormal levels of white blood cells relative to red blood cells and platelets, are performed. In some cases, a bone marrow biopsy is performed to diagnose and/or identify the type of leukemia. Once a diagnosis is made, the leukemia can also be staged. For example, the stages of CLL, the most common type of leukemia in adults older than 19 years of age, are as follows:

Stage 0 is when the blood has too many white blood cells (lymphocytes), but other blood counts are close to normal. There are usually no other symptoms of leukemia. The cancer is slow growing, and this stage is low risk.

Stage I is a medium-risk stage when the blood has too many lymphocytes. At this stage, the lymph nodes are larger than normal, although other organs are normal size. Typically, the red blood cell and platelet counts are close to normal, too.

Stage II is a medium-risk stage when the blood has too many lymphocytes and the spleen is swollen or enlarged. The lymph nodes may also be larger than normal. Red blood cell and platelet counts are close to normal.

Stage III is a high-risk stage when the blood has too many lymphocytes and the patient is anemic (i.e., too few red blood cells). In addition, the lymph nodes, liver, or spleen may be larger than normal. Platelet counts are close to normal.

Stage IV is a high-risk stage when the blood has too many lymphocytes and also has too few platelets. At this stage, the lymph nodes, liver, or spleen may be larger than normal and the patient may be anemic.

Diagnosing lymphoma usually involves a lymph node biopsy. In some cases, an X-ray, blood tests, a CT scan, and/or a PET scan can be used to detect swollen lymph nodes. Once a diagnosis is made, the lymphoma can also be staged. The stages for lymphoma are as follows:

Stage 1 involves only one region or site, such as the lymph nodes or lymph structure.

Stage 2 involves two or more lymph node regions or two or more lymph node structures. At this stage, the involved areas are on the same side of the body.

Stage 3 involves lymph node regions, and structures are on both sides of the body.

Stage 4 involves other organs besides the lymph nodes, and lymph structures are involved throughout the body. These organs may include bone marrow, liver, or lungs.

For diagnosis of myeloma, one or more of a CBC test, blood test, urine test, bone marrow biopsy, X-ray, MRI, PET, and CT scan can be used to confirm the presence and extent of myeloma.

II.B.4.iii. Treatment

Treatment for blood cancer will depend on the type and stage of cancer, as well as the spread of the disease and other basic health parameters. Treatment options include radiation therapy, chemotherapy, immunotherapy, and stem cell transplant.

II.B.4.iii.a. B-Cell lymphomas

B-cell lymphomas make up most (about 85%) of the non-Hodgkin's lymphomas (NHL) in the United States. DLBCL, FL, and CLL are among the most common types of B-cell lymphoma.

II.B.4.iii.b. Diffuse Large B-Cell Lymphoma (DLBCL)

Although treatment of DLBCL will vary depending on the stage and sub-indication of DLBCL, the standard of care for most patients is R-CHOP (rituximab (Mabthera/Rituxan (Roche, Basel, Switzerland)), cyclophosphamide, hydroxydaunorubicin, vincristine and prenisolone) chemotherapy.

Therapies for a first relapse of DLBCL are typically based on whether the intention is to proceed to autologous-stem cell transplant. For patients where the intention is to transplant, typical regimens are R-ICE (rituximab, ifosfamide, carboplatin, and etoposide) and R-DHAP (rituximab, dexamethasone, high-dose cytarabine, and cisplatin) or less commonly R-ESHAP (rituximab, etoposide, solu-medrone, high dose cytarabine, and cisplatin). Other regimens (R-Benda (rituximab and bendamustine) and R-Borte (rituximab and bortezomib)) are typically reserved for patients who are not eligible for a transplant due to factors such as age and presence of co-morbid conditions. In some cases, polaztuzumab vedotin (Polivy® (Roche, Basel, Switzerland)), in combination with bendamustine, plus rituximab, are administered to adult patients with relapsed or refractory DLBCL who are not candidates for a stem cell transplant. If there is a second relapse of DLBCL, R-ICE, R-ESHAP, BR, R-Benda, R-DHAP, or R-Hyper-CVAD (rituximab, hyperfractionated cyclophosphamide, doxorubicin, vincristine, and dexamethasone) can be administered.

II.B.4.iii.c. Follicular Lymphoma (FL)

Although treatment of FL will vary depending on the sub-indication of FL, standard of care, first-line chemotherapy treatments include rituximab (R), R-CHOP (rituximab, cyclophosphamide, hydroxydaunorubicin, vincristine, and prenisolone) chemotherapy, R-Benda, and R-CVP (rituximab, cyclophosphamide, vincristine, and prednisolone). First-line maintenance therapy for FL is usually rituximab.

If a first relapse of FL occurs, patients typically receive a regimen, for example, R-CHOP, R-CVP, R-Benda, or R-DHP, that is different from the first-line therapy. If a second relapse occurs, R-Benda, R-ICE, or idelalisib can be administered to the patient.

In some cases, tazemetostat can be administered to patients with relapsed or refractory FL whose tumors are positive for an enhancer of zeste homolog 2 (EZH2) gene mutation, and who have received at least two prior systemic therapies. FDA-approved tests for detection of an EZH2 mutation are available; for example, the Cobas® EZH2 mutation test (Roche, Basel, Switzerland) can be used to identify mutations in DNA extracted from formalin-fixed paraffin embedded human FL tumor tissue.

II.B.4.iii.d. Chronic Lymphocytic Leukemia (CLL)

CLL is commonly diagnosed in the elderly, with the median age at diagnosis being 72 years. Due to this, at the International Workshop for CLL in 2013, the fitness of patients with CLL was proposed to be a better determinant for patient selection and for identifying treatment goals. Said classification of fitness is necessary because it can: (1) accurately categorize a patient's life expectancy unrelated to CLL (i.e., other health problems); (2) determine the patient's ability to tolerate aggressive chemotherapy, which includes the prediction of treatment modifications and discontinuation; and (3) allow for more consistent stratification and selection of patients across clinical trials. Researchers now recognize the wide heterogeneity of the disease due to the underlying tumor biology (e.g., deletions of 17p and 11q). (Fit vs. Frail Assessment Strategies in CLL, New Evidence Oncology Issue-October 2015). For CLL, patients are treated according to their health condition (fit or unfit), whether they carry certain mutations, and whether they are treated for the first occurrence of the disease or a relapse.

Although treatment of CLL will vary, often a patient's condition will be monitored without administering treatment, until signs or symptoms appear or change. Once the decision is made to administer treatment, options include radiation therapy, chemotherapy, and targeted therapy.

Depending on the sub-indication of CLL, FCR (fludarabine, cyclophosphamide, and rituximab) is often used as a standard-of-care, first-line chemotherapy regimen for fit patients. For patients with a history of previous infections, Benda-R can be used. An alternative first-line option, for those less fit, is a combination of chlorambucil and an anti-CD20 antibody (e.g., rituximab, ofatumumab, or obinutuzumab). For patients with a TP53 mutation or a del(17p) mutation, a BCR receptor antagonist with or without rituxamib can be administered. Alternatively, a hematopoietic stem cell transplant can be considered for patients in remission.

If a patient has relapsed or refractory CLL, a BCL2 antagonist with or without rituximab can be administered to the patient. Alternatively, R-Benda or FCR can be administered to the patient. Other regimens for a relapsed CLL include ibrutinib, idelalisib and rituximab, or an allogeneic hematopoieitic stem cell transplant. In cases where a patient has relapsed CLL and has a TP53 mutation or a del(17p) mutation, a BCL2 antagonist with or without rituximab can be administered to the patient. Alternatively, other regimens include ibrutinib, idelalisib and rituximab, or an allogeneic hematopoieitic stem cell transplant.

Supportive care regimens can also be administered to patients being treated or who have been treated for cancer. These include medications for chemotherapy- and/or radiotherapy-induced nausea and vomiting (e.g., Kytril® (Roche, Basel, Switzerland)); anti-anemia medications (e.g., NeoRecorman (Roche, Basel, Switzerland)); medications to treat or prevent bone metastasis (e.g., Bondronat® (Roche, Basel, Switzerland)); and treatment for neutropenia (e.g., Neupogen® (Roche, Basel, Switzerland)), to name a few.

### III. Overview of Cloud-Based Network Architecture for Deploying Intelligent Functionality

Techniques relate to configuring a server to execute code that enables a user (e.g., a physician) of an entity to execute machine-learning or AI techniques using subject records. Subject records include a complex combination of data elements that characterize subjects. As an illustrative example, a subject record may include a combination of thousands of data fields. Some data fields may contain fixed non-numerical values (e.g., a subject's ethnicity), other data fields may contain unstructured text data (e.g., notes prepared by a physician), other data fields may include a time-variant series of collected measurements (e.g., glycosylated hemoglobin measurements taken two to four times a year), and other data fields may include images (e.g., MRI of a subject's brain). The complexity and variance of data types and formats in subject records make processing subject records technically challenging, if not impossible, because machine-learning and AI models are often configured to process data in numerical or vector form. In light of this objective technical problem, certain aspects and features of the present disclosure relate to transforming subject records into transformed representations, such as vector representations, that characterize the various data elements of the subject records.

Techniques relate to transforming the non-numerical values included in subject records into numerical representations (e.g., feature vectors) that can be inputted into machine-learning or AI models to generate predictive outputs. The server executing the code provides a technical effect, which solves the objective technical problem, by transforming the subject records into transformed representations that are consumable by machine-learning or AI models. “Consumable” may refer to data that is in a format or form that machine-learning or AI models are configured to process to generate predictive outputs. Machine-learning or AI models are not configured to process subject records (as they exist in their stored state in the data registries) due to the complex combinations of data elements in multiple data formats and data types contained in each individual subject record. To illustrate, for a given subject record, a data element may include a longitudinal sequence of events (e.g., an immunization record), another data element may include measurements taken from a subject (e.g., vitals), yet another data element may include text entered by the user (e.g., notes taken by the physician), and yet another data element may be an image (e.g., an X-ray). A limited or simplistic analysis may be performed on subject records (before any transformations), such as grouping subjects based on a value of a data element (e.g., age group). However, the limited or simplistic analysis becomes problematic or infeasible as the complexity and size of subject records reaches a big-data scale. To process and extract analytical assessments from the subject records at a big-data scale, machine-learning or AI techniques can be used for data mining the subject records. Machine-learning or AI models, however, are configured to receive numerical or vector inputs. For example, clustering operations, such as k-means clustering, are configured to receive vectors as inputs. Thus, to perform the clustering operation on subject records, the present disclosure provides a technical effect, which solves the objective technical problem by transforming the subject records into transformed representations, such as numerical vector representations, that are consumable by machine-learning or AI models. An intelligent analysis can be performed on subject records in their transformed representation state. Non-limiting examples of intelligent analysis (performed upon the server executing code) may include automatically detecting subject groups using clustering techniques, generating outputs predictive of certain outcomes based on the values of data elements in subject records, and identifying existing subject records that are similar to a given or new subject record.

To illustrate and only as a non-limiting example, a subject record of a subject includes four data elements. The first data element contains a unique code that represents a diagnosis of a condition. The second data element contains an MRI of the subject's brain. The third data element contains a time-variant series of measurements, such as blood pressure readings, over the course of one year. The fourth data element contains unstructured notes, for example, notes of a condition detected by examining or running one or more tests. According to certain implementations, each of the first data element, the second data element, the third data element, and the fourth data element may be transformed into a transformed representation (e.g., a vector). The techniques used for transforming the values contained within the four data elements may depend on the type of data contained in a data element. For the first data element, for example, the unique code that represents a diagnosis can be represented as a fixed-length vector, such that the size of the vector is determined by a size of a vocabulary of codes, and that each code in the vocabulary is represented by a vector element of the fixed-length vector. The one or more unique codes contained within the first data element may be compared with the vocabulary of codes. If a unique code matches a code of the vocabulary, then a “1” may be assigned to the vector element at the position of the vector that corresponds to the unique code and a “0” may be assigned to all remaining vector elements of the vector. In light of the above, a first vector may be generated to represent the value of the first data element. As another example, for the second data element, a latent-space representation of the image may be generated using a trained auto-encoder neural network. The latent-space representation of the input image may be a reduced-dimensionality version of the input image. The trained auto-encoder neural network may include two models: an encoder model and a decoder model. The encoder model may be trained to extract a subset of salient features from the set of features detected within the image. A salient feature (e.g., a key point) may be a region of high intensity within the image (e.g., an edge of an object). The output of the encoder model may be a latent-space representation of the input image. The latent-space representation may be outputted by a hidden layer of the trained auto-encoder model, and thus, the latent-space representation may only be interpretable by the server. The decoder model may be trained to reconstruct the original input image from the extracted subset of salient features. The output of the encoder model may be used as the feature vector that represents the pixel values of the image included in the second data element. In light of the above, a second vector (e.g., the latent-space representation) may be generated to represent the image contained in the second data element. As another example, for the third data element, the time-variant sequence of measurements can be represented numerically. In some implementations, the time-variant sequence can be represented by a total of the instances a measurement was taken from a subject. In other implementations, the time-variant sequence can be represented numerically using an average, mean, or median of the values of the measurements taken across the instances of measurements that occurred during a time period (e.g., one year). In other implementations, a frequency of measurements can be calculated and used to numerically represent the time-variant sequence of measurements. In light of the above, a third vector may be generated to represent the time-variant sequence of values contained within the third data element. As yet another example, for the fourth data element, the notes inputted by the user may be processed and vectorized using any number of natural-language processing (NLP) text vectorization techniques. In some implementations, a word-to-vector machine-learning model, such as a Word2Vec model, may be executed to transform the notes contained in the fourth data element into a single vector representation. In other implementations, a convolutional neural network may be trained to detect words or numbers within text that indicate symptoms, treatments, or diagnoses from the notes contained in the fourth data element. In light of the above, a fourth vector may be generated to represent the text of the notes contained in the fourth data element as a vector representation. Thus, the final feature vector that represents the entire subject record may be a vector of vectors, including a concatenation of the first vector, the second vector, the third vector, and the fourth vector. In other examples, an average of the first vector, the second vector, the third vector, and the fourth vector may be used to numerically represent the entire subject record. Other combinations of the first vector, second vector, third vector, and fourth vector may be used to generate the final feature vector that numerically represents the entire subject record.

In some implementations, instead of generating a vector to numerically represent each data element of a subject record, techniques may be executed to reduce the dimensionality of the subject record by identifying and selecting a subset of data elements from the set of data elements. The subset of data elements may represent the “important” data elements, where “importance” of a data element is determined based on a prediction using feature extraction techniques, such as singular value decomposition (SVD). For example, transforming a subject record into a transformed representation that is consumable by machine-learning and AI models may include performing one or more feature extraction techniques on the non-numerical values included in the data elements of a subject record to generate a feature vector that numerically represents a decomposed version of the non-numerical values. In some implementations, feature extraction techniques may include, for example, reducing the dimensionality of a set of data elements of a subject record (e.g., each data element representing a feature or dimension of a subject) into an optimal subset of features that can be used to, for example, predict an outcome or event. Reducing the dimensionality of the set of data elements may include reducing N data elements into a subset of M elements, where M is smaller than N. In these implementations, each element of the subset of M elements may be transformed into a numerical value. In some implementations, a feature vector may be generated to represent the N data elements of a subject record. The feature vector may include a vector for each data element of the set of data elements. For example, the feature vector may be a numerical representation of the complex combinations of data elements of a subject record. Each non-numerical value in a data element of a subject record can be vectorized to generate a representative vector. The vectors representing the set of data elements in a subject record may be concatenated or combined (e.g., as an average or weighted average) to generate the feature vector that numerically characterizes the entire set of data elements of the subject record. The feature vector is consumable by a trained machine-learning or AI model. Once the feature vector for a subject record is generated, the subject record can be evaluated individually or in groups of other subject records using machine-learning and AI techniques. After the feature vector that represents each subject record has been generated and stored, the feature vectors of the subject records stored in a central data store can be inputted into machine-learning or AI models, or other enhanced analyses can be performed on the numerical representations of the subject records. For example, two different subject records can be compared with respect to one or more dimensions. A dimension may represent a feature or data element of a subject record, along which a comparison between two or more subject records is made. To illustrate, a data element of a first subject record contains text inputted by a first user (e.g., a doctor) describing symptoms of a first subject. The text (e.g., the value of the data element of the first subject record) can be vectorized using the text vectorization techniques (e.g., Word2Vec) described above to generate a first vector to numerically represent the text associated with the data element. The text vectorization technique may generate an N-dimensional word vector for each word included in the text. The matching data element of a second subject record (e.g., the data element of another subject record that also contains text inputted by a physician describing symptoms of another subject) may contain text inputted by a second user describing the symptoms of a second subject. The text (e.g., the value of the data element of the second subject record) can be vectorized using the text vectorization techniques described above to generate a second vector (e.g., an N-dimension word vector) to represent the text associated with the data element. A server may compare the first vector with the second vector in a Euclidean or cosine space to quantify a similarity or dissimilarity between the first subject record and the second subject record, at least with respect to the dimension of a subject's presentation of symptoms. If the first vector and the second vector are near each other (or within a threshold distance) in the Euclidean space (i.e., if the Euclidean distance between the first vector and the second vector is small), then the symptoms experienced by the first subject (as described in the text of the data element) are likely similar to the symptoms experienced by the second subject (as described in the text of the data elements). However, if the Euclidean distance between the first vector and the second vector is large or above the threshold distance (e.g., or if the Euclidean distance is above a threshold), then the symptoms experienced by the first subject can be predicted to be different from the symptoms experienced by the second subject.

In some implementations, a server may be configured to execute an application that enables a user of an entity to build data registries that serve to store subject records for subsequent processing. The data of a subject record may include unstructured data, such as electronic copies of physician notes and/or responses to open-ended questions. The unstructured data can be ingested into the data registries by mapping portions of the unstructured data to fixed parts (e.g., data elements) of structured data records. The structure of the structured data records may be defined using, for example, specifications from a module that corresponds to a particular use case (e.g., particular disease, particular trial). For example, each word of the unstructured note data (i.e., text) may be transformed into a numerical representation and the various numerical representations associated with the unstructured note data can be decomposed (e.g., using SVD) to detect words describing a particular set of symptoms that the subject has exhibited. The decomposition of the numerical representations of the unstructured note data may remove non-informative words, such as “and,” “the,” “or,” and so on. The remaining words represent the particular set of symptoms. Some portions of the note data may be irrelevant with regard to data elements in the structured data and/or may be more or less specific than data contained in data elements. In some instances, various mapping (e.g., mapping a “poor balance” symptom to a “neurological” symptom), NLP, or interface-based approaches (e.g., that requests new information from a user) can be used to obtain structured data records. An interface may also be used to receive input that identifies new information about anew or existing subject, and the interface may include input components and selection options that map to a structure of data records.

Further, techniques relate to configuring a cloud-based application to transform non-numerical values contained in data elements of subject records into numerical representations, so that the cloud-based application can execute intelligent analytical functionality using the numerical representations (e.g., the transformed representations) of the subject records stored in the data registries. The transformation of non-numerical values of data elements of subject records to numerical representations may be dependent on the type of data contained in a data element. For example, for data elements that include text, such as notes taken by a user, the text may be transformed into numerical representations of the text using NLP techniques, such as Word2Vec or other text vectorization techniques. As another example, for data elements that include images (e.g., MRIs) or image frames of a video (e.g., a video of an ultrasound), each image or image frame may be transformed into a numerical representation (e.g., vector) using a trained auto-encoder neural network, which is trained to generate a latent-space representation of an input image. The condensed representation of the input image (e.g., the latent-space representation) may serve as the vector that numerically represents the input image. As yet another example, for data elements that include a time-variant sequence of information (e.g., events occurring over a period of time), the time-variant information can be represented as a numerical representation using several exemplary transformations. In some instances, the count of events may be used as the vector representing the time-variant information. In other instances, the frequency or rate of events occurring (e.g., per week, per month, per year) may be used as the vector representing the time-variant information. In still other instances, an average or combination of the measurement values associated with each event in the time-variant information can be used as the vector representing the time-variant information. The present disclosure is not limited to these examples, and thus, other numerical representations of time-variant information can be used as the vector that represents the numerical representation. Intelligent analytical functionality may be performed by executing trained machine-learning or AI models using data records. The model outputs may be used to indicate certain analytics extracted from the data records.

In some instances, transmission of data from a subject record may be provided to develop a treatment plan for an individual subject. For example, subject-record information (e.g., that complies with data-privacy restrictions via, for example, select omission and/or obscuring of data) may be broadcast and/or transmitted to a select group of user devices. For example, a broadcast may be transmitted to user devices associated with similar data records in response to input from the user corresponding to a request to initiate a consult with a user associated with a similar subject. If a user receiving the broadcast accepts a consultation request (via provision of corresponding input), a secure data channel may be established between the users, and potentially more of the subject record may be shared (e.g., while conforming to data-privacy restrictions applicable to the two users). Subject records that are similar to a given subject may be identified by performing a nearest-neighbor technique using the vector representations of two or more subject records. Nearest-neighbor techniques may be performed by comparing vectors of individual data elements across multiple subject records (e.g., the nearest neighbor may be determined in association with a dimension or feature of the subject records). Alternatively, the nearest-neighbor techniques may be performed by comparing the overall vector that characterizes the entire subject record with the overall vector that characterizes another entire subject record. An overall vector may be a concatenation of individual vectors representing the values of the data elements, or may be an average or combination of the individual vectors representing the values of the data elements.

As another example, one or more processed data records may be returned in response to a query for subject records matching particular constraints. In some instances, a first user may submit a query that identifies a first subject record. The query may correspond to a request to identify other subject records that are similar to the first subject record. A server may transform the first subject record into a transformed representation using certain transformation techniques, discussed above and herein. Alternatively, the transformed representation of the first subject record may have previously been generated and stored in a database. Regardless of whether the transformed representation of the first subject record is generated before or after the query is received, transforming the first subject record into a transformed representation of the first subject record may include generating a vectorization of one or more non-numerical values of data elements of the first subject record. Vectorizing the one or more non-numerical values contained within the first subject record may include generating a numerical vector representation for each value (e.g., for non-numerical text, such as notes) included in each data element of the first subject record. The various vector representations may be concatenated or otherwise combined (e.g., an average may be computed) to generate the feature vector that represents the entire first subject record. The vector representation that numerically represents the first subject record may be compared in a domain space (e.g., Euclidean space or cosine space) to vector representations of other subject records. When the Euclidean distance, for example, between two vector representations is within a threshold distance, then the two subject records associated with the two vector representations may be interpreted (e.g., by a server) as being similar, at least with respect to one or more dimensions.

For each data element in a subject record, the technique used to generate the vector representation of the value associated with the data element may depend on the type of data associated with the data element. In some examples, the data element of a subject record may be associated with one or more images, such as X-rays of the subject. Feature extraction techniques may be executed to generate a vector representation of each image associated with the data element. For example, a server may be configured to execute a trained auto-encoder neural network to generate a reduced-dimensionality version of the image. The trained auto-encoder neural network may include two models: an encoder model and a decoder model. The encoder model may be trained to extract a subset of salient features from the set of features detected within the image. A salient feature (e.g., a key point) may be a region of high intensity within the image (e.g., an edge of an object). The output of the encoder model may be a latent-space representation of the input image. The latent-space representation may be outputted by a hidden layer of the trained auto-encoder model, and thus, the latent-space representation may only be interpretable by the server. The subset of salient features of the latent-space representation that characterizes the subject record can be compared against the subset of salient features of the latent-space representation that characterizes another subject record to yield certain analytical insights. The decoder model may be trained to reconstruct the original input image from the extract subset of salient features. The output of the encoder model may be the vector representation of the data element associated with the image included the subject record. In other examples, key point matching techniques may be executed to match key points of an image contained in a data element of a first subject record to key points of another image contained in a data element of a second subject record. The vector representation (e.g., the latent-space representation) of the input image is consumable by machine-learning or AI models, and thus, two different subject records (each including an image) may be compared against each other to determine a similarity or a dissimilarity between the two different subject records.

To illustrate and only as a non-limiting example, a magnetic resonance image (MRI) of a subject's brain is captured. The MRI is stored in the subject record associated with the subject. The server is configured to generate a transformed representation, such as a vector representation, of the MRI contained in the subject record using feature extraction techniques, such as key point detection, auto-encoding to latent-space representations, SVD, and other suitable computer-vision techniques. The vector representation of the data element that contains the MRI is concatenated or otherwise combined (e.g., averaged) with the vector representations of each remaining data element of the set of data elements to generate the feature vector that characterizes the entire subject record. A user may access an application to query a database of other subject records to retrieve a subset of other subject records that contain MRIs that are similar to the MRI of the subject's brain. Identifying other subject records that are similar to the subject record (at least with respect to similarity between MRIs) may involve calculating the k-nearest neighbors of the subject record. For example, the transformed representation may be plotted (visually or internally by a computing system) on a domain space, such as a Euclidean space or cosine space. The transformed representation of each other subject record may also be plotted (visually or internally by a computing system). A nearest-neighbor technique may be executed to compare the vector representation of the subject record with the vector representations of the other subject records to identify the k-nearest neighbors to the subject vector. The k-nearest neighbors that are identified may be predicted to have MRIs that are similar to the MRI of the subject's brain. Each other subject record that is identified as a nearest neighbor may be identified and retrieved for further evaluation or processing using the application.

In some implementations, a computing system may perform a data-processing technique (e.g., nearest-neighbor technique) to identify similar subject records. Various data elements may be differentially weighted in this search (e.g., in accordance with predefined data element weightings, user input that indicates an importance of matching various data elements, and/or a prevalence of particular data element values across a subject record set). When searching across a set of records for potential matches, some records may lack values for various data elements. In these cases, it may be determined that (for example) the data element values do not match and/or the data element may be unweighted when evaluating the potential match. Handling of the missing value may depend on a distribution of values for the data element across the set of records and/or the value for the data element in the query.

Further, some techniques relate to defining and using a set of rules used to identify potential treatment regimens for a subject given a set of symptoms identified in the subject record. To illustrate, a target subject record may represent a target subject who recently experienced three symptoms: an upper respiratory infection, a fever, and a sore throat. The three symptoms may be written as text within a data element of the target subject record (e.g., the separation between words being marked by a tag, such as a semicolon). A server, such as cloud server 135, may individually input the text “upper respiratory infection,” “fever,” and “sore throat” into a trained Word2Vec model or other text-to-vector model, such as vocabulary mapping. The Word2Vec model may be trained to generate a vector representation for each word that represents a symptom. The vector representations for the three symptoms may be averaged to generate a single vector representation for the “symptoms” data element of the target subject record. The single vector representation for the “symptoms” data element of the target subject record may be processed to identify other subject records that include similar words in the “symptoms” data element. Each subject record stored in the database may be associated with an existing “symptoms” data element that has been transformed into a numerical representation, such as a vector. The vector for the “symptoms” data element may be plotted and compared against the vector for the “symptoms” data element of the target subject record. The server may identify the nearest vector to the vector characterizing the “symptoms” data element. The vector of the “symptoms” data element nearest the vector of the target subject record may be predicted to be similar to the subject. The subject record associated with the nearest vector to the vector of the target subject record may be identified and further evaluated to determine the treatment regimen provided to that subject. The treatments that were provided to the subject associated with the vector nearest the vector for the target subject record may be used as potential treatment regimens to treat the target subject. Additionally, each potential treatment regimen may be weighted by the responsiveness experienced by other subject. The potential treatment regimens may be sorted according to the responsiveness that the other subject experienced.

A set of rules may be defined based on a user interaction with a user interface, which may include specifications of particular criteria and an associated particular medical treatment and/or selection of one or more previously defined rules (that specify criteria and a treatment). For example, one or more existing rules may be presented via an interface, and a user may select rules to incorporate into a rule base associated with an account associated with the user. The one or more rules may be selected from amongst a set of rules defined by multiple users (e.g., associated with one or more institutions) and/or may be generated based on rules generated by multiple users. When a user selects a rule for incorporating into a rule base, the application may generate a feedback signal to cloud server 135. The feedback signal may include metadata associated with the user's selection. The metadata may indicate whether the rule was incorporated into the rule base without modification or with modification. If the rule base was modified, then the metadata would indicate which modification was made to the rule. The metadata may also indicate whether the rule was rejected, deleted, or otherwise determined not to be useful to the user. To illustrate and as a non-limiting example, a computing system may detect that rules that relate one or more particular types of symptoms and/or test results to a given treatment are relatively frequently defined and/or selected by users, and the computing system may then generate a general rule pertaining to the particular types of symptoms and/or test results and to the treatment. The general rule may be defined to have (for example) a most restrictive, most inclusive, or median criteria. In some instances, a rule base of a user can be processed to detect any criteria overlap between rules. Upon identifying an overlap, an alert may be presented that identifies the overlap. A rule of a rule base may be used to evaluate a subject record to classify to define a population associated with the subject record. Evaluating the subject record using the rule may be performed as a decision tree, for example, in that a first criterion of the rule is compared against the attributes included in the subject record. If the first criterion is satisfied, then the next criterion is compared against the attributes included in the subject record. If the next criterion is satisfied, then the comparisons continue for each criterion included in the rule. The comparisons may continue even if the next criterion is not satisfied. In this case, the non-satisfaction of the criterion (and any others included in the rule) is stored and presented to a user device, along with the criteria that were satisfied.

Accordingly, embodiments of the present disclosure provide a cloud-based application configured to exchange subject information with external entities without violating data-privacy rules. The cloud-based application is configured to automatically assess data-privacy rules involved in sharing subject information across various jurisdictions. The cloud-based application is configured to execute protocols that obfuscate or otherwise modify the subject information, thereby algorithmically ensuring compliance with the data-privacy rules.

### IV. Network Environment for Hosting the Cloud-Based Application Configured with Intelligent Functionality

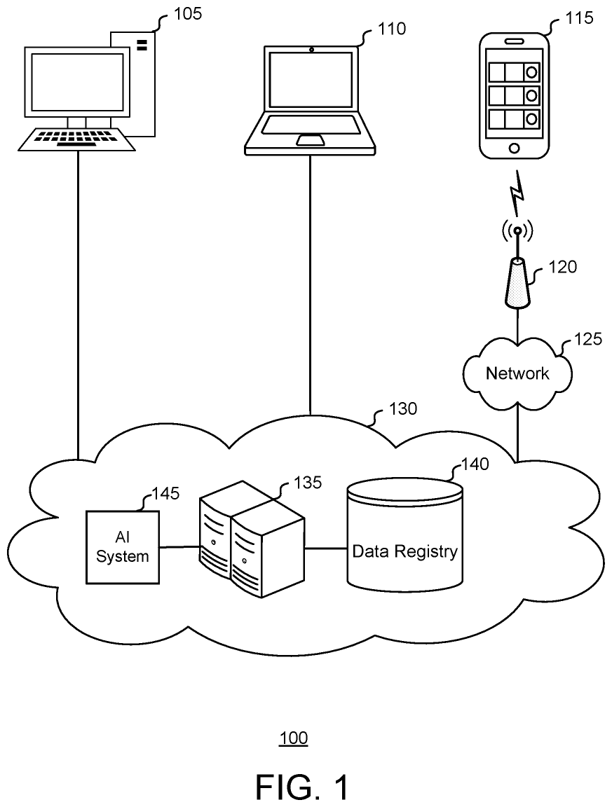

FIG. 1 illustrates network environment 100, in which an embodiment of the cloud-based application is hosted. Network environment 100 may include cloud network 130, which includes cloud server 135, data registry 140, and AI system 145. Cloud server 135 may execute the source code underlying the cloud-based application. Data registry 140 may store the data records ingested from or identified using one or more user devices, such as computer 105, laptop 110, and mobile device 115.

The data records stored in data registry 140 may be structured according to a skeleton structure of fixed parts (e.g., data elements). Computer 105, laptop 110, and mobile device 115 may each be operated by various users. For example, computer 105 may be operated by a physician, laptop 110 may be operated by an administrator of an entity, and mobile device 115 may be operated by a subject. Mobile device 115 may connect to cloud network 130 using gateway 120 and network 125. In some examples, each of computer 105, laptop 110, and mobile device 115 is associated with the same entity (e.g., the same hospital). In other examples, computer 105, laptop 110, and mobile device 115 are associated with different entities (e.g., different hospitals). The user devices of computer 105, laptop 110, and mobile device 115 are examples for the purpose of illustration, and thus, the present disclosure is not limited thereto. Network environment 100 may include any number or configuration of user devices of any device type.

In some embodiments, cloud server 135 may obtain data (e.g., subject records) for storing in data registry 140 by interacting with any of computer 105, laptop 110, or mobile device 115. For example, computer 105 interacts with cloud server 135 by using an interface to select subject records or other data records stored locally (e.g., stored in a network local to computer 105) for ingesting into data registry 140. As another example, computer 105 interacts with an interface to provide cloud server 135 with an address (e.g., a network location) of a database storing subject records or other data records. Cloud server 135 then retrieves the data records from the database and ingests the data records into data registry 140.

In some embodiments, computer 105, laptop 110, and mobile device 115 are associated with different entities (e.g., medical centers). The data records that cloud server 135 obtains from computer 105, laptop 110, and mobile device 115 may be stored in different data registries. While the data records from each of computer 105, laptop 110, and mobile device 115 may be stored within cloud network 130, the data records are not intermingled. For example, computer 105 cannot access the data records obtained from laptop 110 due to the constraints imposed by data-privacy rules. However, cloud server 135 may be configured to automatically obfuscate, obscure, or mask portions of the data records when those data records are queried by a different entity. Thus, the data records ingested from an entity may be exposed to a different entity in an obfuscated, obscured, or masked form to comply with data-privacy rules.

Once the data records are collected from computer 105, laptop 110, and mobile device 115, the data records may be used as training data to train machine-learning or AI models to provide the intelligent analytical functionality described herein. The data records may also be available for querying by any entity, given that when a user device associated with an entity queries data registry 140 and the query results include data records originating from a different entity, those data records may be provided or exposed to the user device in an obfuscated form, which complies with data-privacy rules.

Cloud server 135 may be configured in a specialized manner to execute code that, when executed, causes intelligent functionality to be performed using transformed representations of subject records (e.g., a vector that numerically represents the information stored in a subject record). For example, intelligent functionality may be performed by executing code using cloud server 135. The executed code may represent a trained neural network model. The neural network model may have been trained to perform intelligent functions, such as predicting a subject's responsiveness to a treatment regimen, identifying similar patients, generating a recommendation of a treatment regimen for a patient, and other intelligent functionality. The neural network model may be trained using a training data set that includes subject records of subjects who have previously been treated for a condition and experienced an outcome (e.g., overcoming a condition, increasing a severity of a condition, reducing a severity of a condition, and so on). Additionally, the executed code may be configured to cause cloud server 135 to transform non-numerical values of existing subject records into numerical representations (e.g., a transformed representation), which can be processed by the trained neural network model. For example, the code executed by cloud server 135 can be configured to receive as input each subject record of a set of subject records, and for each subject record, the code, when executed, can cause cloud server 135 to perform the operations described herein for transforming each data element of each subject record into a transformed representation, such as a vector representation. Executing intelligent functionality may include inputting at least a portion of the data records stored in data registry 140 into a trained machine-learning or AI models to generate outputs for further analysis. In some embodiments, the outputs can be used to extract patterns within the data records or to predict values or outcomes associated with data fields of the data records. Various embodiments of the intelligent functionality executed by cloud server 135 are described below.

In some embodiments, cloud server 135 is configured to enable a user device (e.g., operated by a doctor) to access the cloud-based application to transmit consult broadcasts to a set of destination devices. A consult broadcast may be a request for support or assistance regarding the treatment of a subject associated with a subject record. A destination device may be a user device operated by another user associated with another entity (e.g., a doctor at another medical center). If a destination device accepts the request for assistance associated with the consult broadcast, the cloud-based application may generate a condensed representation of the subject record that omits or obscures certain data fields of the subject record. The condensed representation may comply with data-privacy rules, and thus, the condensed representation of the subject record cannot be used to uniquely identify the subject associated by the subject record. The cloud-based application may transmit the condensed representation of the subject record to the destination device that accepted the request for assistance. The user operating the destination device may evaluate the condensed representation and communicate with the user device using a communication channel to discuss options for treating the subject. For example, the communication channel may be configured as a secure chatroom that enables the user device (e.g., operated by the doctor requesting the consult) to securely communicate with the destination device (e.g., operated by the other doctor providing the consult).

In some embodiments, cloud server 135 is configured to provide a treatment-plan definition interface to user devices. The treatment-plan definition interface enables user devices to define a treatment plan for a condition. For example, a treatment plan may be a workflow for treating a subject with the condition. A workflow may include one or more criteria for defining a population of subjects as having the condition. The workflow may also include a particular type of treatment for the condition. The cloud server 135 receives and stores treatment-plan definitions for a particular condition from each user device of a set of user devices. The cloud-based application may distribute a treatment plan for a given condition to a set of user devices. Two or more user devices of the set of user devices may be associated with different entities. Each of the two or more users devices may be provided with the option to integrate any portion or the entire treatment plan into a customer rule set. Cloud server 135 can monitor whether user devices integrate the shared treatment plan in full or integrate part of the treatment plan. The interactions between the user devices and the shared treatment plan can be used to determine whether to update the treatment plan or a rule created based on the treatment plan.

In some embodiments, cloud server 135 enables a user operating a user device to access the cloud-based application to determine a proposed treatment for a subject with a condition. The user device loads an interface associated with the cloud-based application. The interface enables the user operating the user device to select a subject record associated with a subject being treated by the user. The cloud-based application may evaluate other subject records to identify a previously treated subject who is similar to the subject being treated by the user. The similarity between subjects, for example, may be determined using an array representation of the subject records. An array representation (e.g., a transformed representation, such as a vector, an N-dimensional matrix, or any numerical representation of a non-numerical value) may be any numerical and/or categorical representation of the values of data fields of a subject record. For example, an array representation of a subject record may be a vector representation of the subject record in a domain space, such as in a Euclidean space. In some instances, cloud server 135 may be configured to transform an entire subject record into a numerical representation, such as a vector. For a given subject record, cloud server 135 may evaluate each data element to determine the type of data contained or included in that data element. The type of data may inform the cloud server 135 as to which process or technique to perform to transform the numerical or non-numerical values of that data element into a numerical representation. As an illustrative example, cloud server 135 may transform non-numerical values (e.g., the text of a physician's notes) of a data element of a subject record into a numerical representation (e.g., a vector). The transformation may include using NLP techniques, such as Word2Vec or other text vectorization techniques, to generate a numerical value that represents each word of text. The generated numerical value may serve as a vector that can be inputted into a trained neural network to perform intelligent analysis. As another illustrative example, for data elements that include images (e.g., MRI data) or image frames of a video (e.g., a video data of an ultrasound), each image or image frame may be transformed into a numerical representation (e.g., vector) using a trained auto-encoder neural network, which is trained to generate a latent-space representation of an input image. The condensed representation of the input image (e.g., the latent-space representation) may serve as the numerical representation of the input image. This numerical representation can be inputted into a neural network or other machine-learning model to perform intelligent analysis of the associated subject record. As yet another example, for data elements that include a time-variant sequence of information (e.g., events occurring or measurements taken from a subject over a period of time), the time-variant information can be represented as a numerical representation using several exemplary transformations. In some instances, the count of events may be used as the vector representing the time-variant information. For example, if a measurement was taken with respect to a subject four times in one year, the numerical representation may be “4.” In other instances, the frequency or rate of events occurring (e.g., per week, per month, per year) may be used as the vector representing the time-variant information. In still other instances, an average or combination of the measurement values associated with each event in the time-variant information can be used as the vector representing the time-variant information. The present disclosure is not limited to these examples, and thus, other numerical representations of time-variant information can be used as the vector that represents the numerical representation.

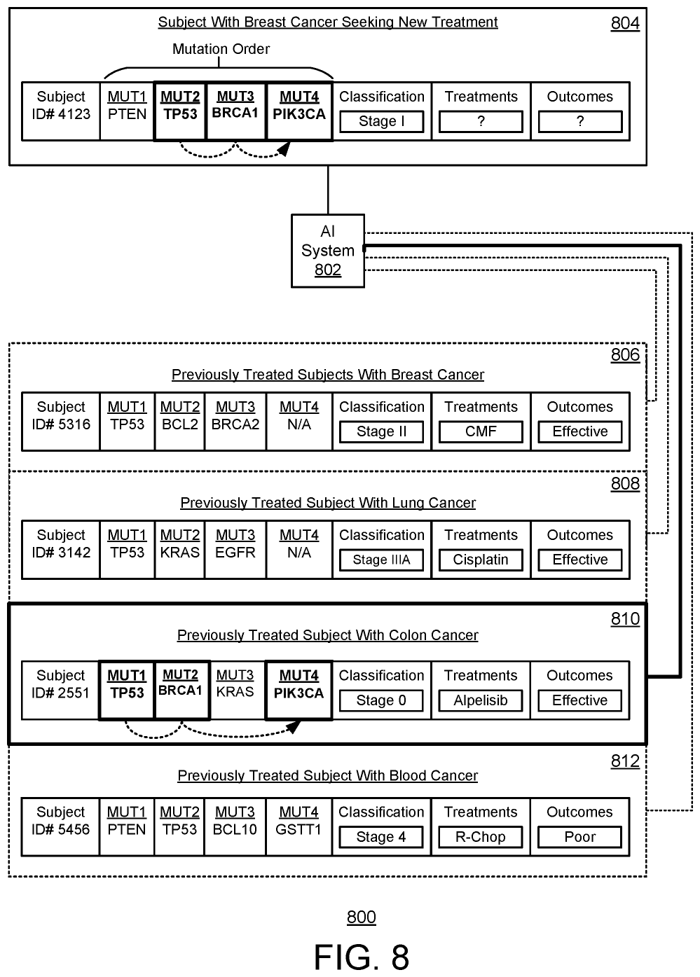

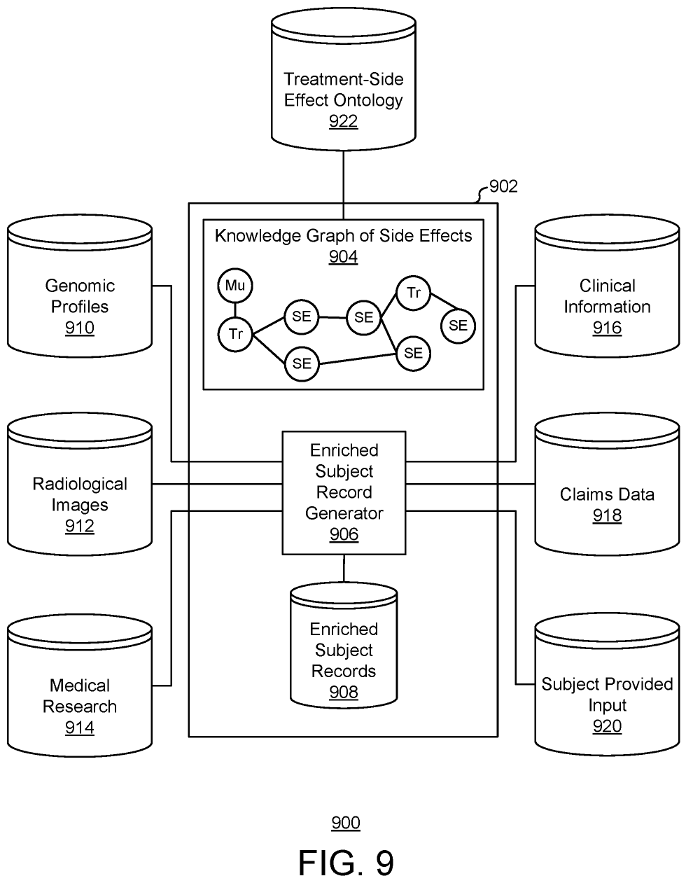

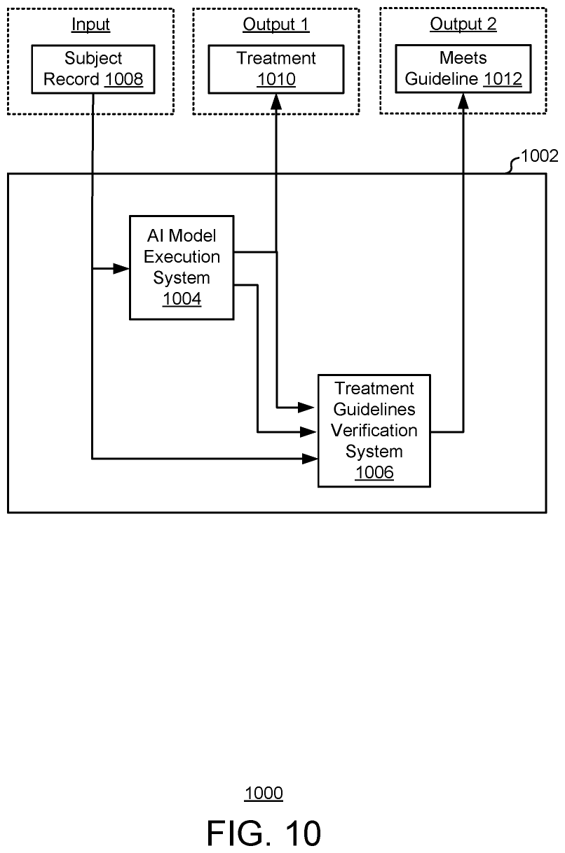

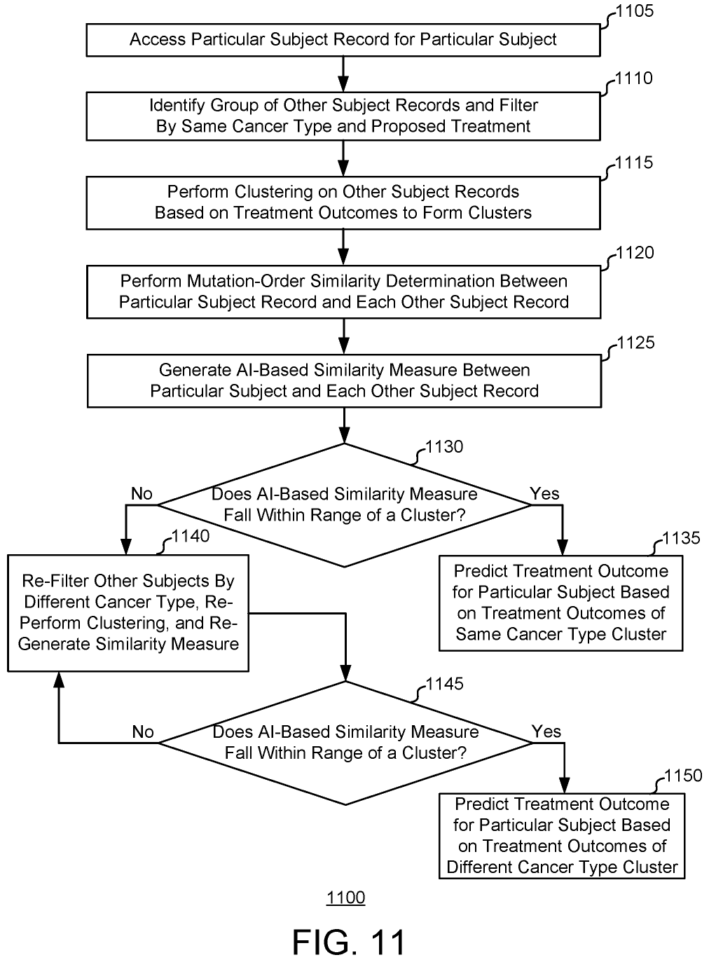

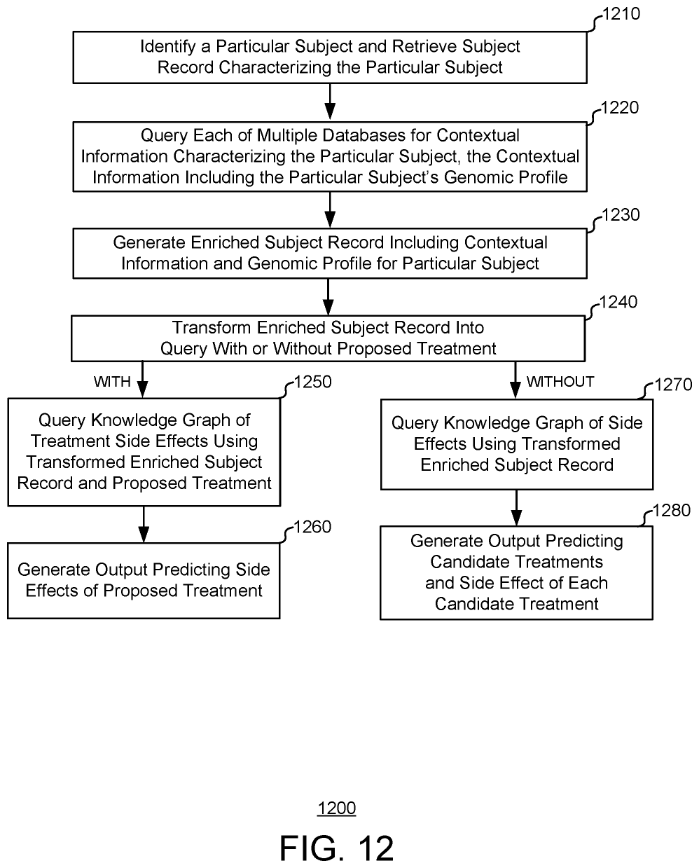

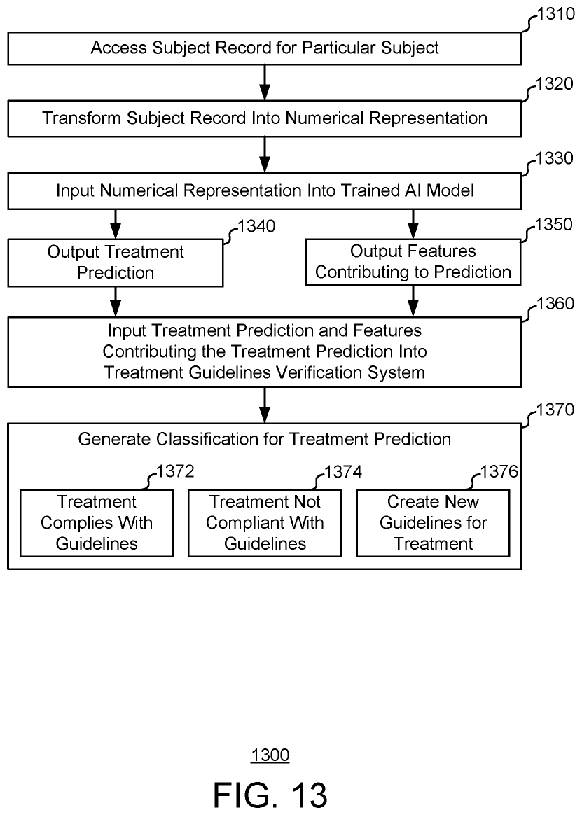

AI system 145 can be configured to collect data sets at a big-data scale, transform the collected data sets into curated training data, execute learning algorithms using the curated training data, and store the detected patterns, correlations, and/or relationships of the training data in one or more trained AI models. In some implementations, AI system 145 can be configured to perform certain predictive functionality, such as predicting therapeutic outcomes and cancer evolution in a particular subject based on mutational profile of subjects across cancer types, predicting treatment survival prospects for a particular subject using enriched subject-specific data sets, and automatically validating whether the features that contribute to the selection of treatments follow oncological guidelines. In some implementations, as described in greater detail with respect to FIGS. 8 and 11, the output of AI system 145 can be predictive of the therapeutic outcomes and/or cancer evolution in a particular subject. In other implementations, as described in greater detail with respect to FIGS. 9 and 12, the output of AI system 145 can be predictive of treatment survival prospects for a particular subject. In other implementations, as described in greater detail with respect to FIGS. 10 and 13, the output of AI system 145 can classify whether the features of a subject that contributed to the selection of a treatment follow existing oncological guidelines.

In some instances, multiple values in an array representation correspond to a single field. For example, a value of a data element may be represented by multiple binary values generated via one-hot encoding. As another example, each value of the multiple values in a single data element of a subject record may be individually transformed into a numerical representation, as described above. The numerical representation that represents each value of the multiple values can be combined into a single numerical representation that corresponds to the data element. Combining multiple numerical representations may be performed using any vector combination techniques, such as averaging vector magnitudes, adding vectors, or concatenating multiple vectors into a single vector. In some instances, the cloud-based application may generate array representations for each subject record of a group of subject records. Similarity between two subject records may be represented by comparing the two array representations to determine a distance between them. Subject records can also be compared along a dimension (e.g., a data element), instead of comparing a numerical representation of an entire subject record with another numerical representation of another subject record. For example, comparing two subject records along a dimension may include comparing the numerical representation of a data element of a subject record with another numerical representation of a matching data element of another subject record. Further, the cloud-based application may be configured to identify a subject who is a nearest neighbor to the subject record selected by the user device using the interface. The nearest neighbor may be determined by comparing the numerical representations of the various subject records with the numerical representation of a target subject record. The cloud-based application may identify treatments previously performed on the subject who is the nearest neighbor. The cloud-based application may avail on the interface the previously performed treatments on the nearest neighbor.

In some embodiments, cloud server 135 is configured to create queries that search a database of previously treated subjects. Cloud server 135 may execute the queries and retrieve subject records that satisfy the constraints of the query. In presenting the query results, however, the cloud-based application may only present the subject record in full for subjects who have been or who are being treated by the user who created the query. The cloud-based application masks or otherwise obfuscates portions of subject records for subjects who are not being treated by the user creating the query. The masking or obfuscation of portions of subject records that are included in the query results enables the user to comply with data-privacy rules. In some embodiments, the query results (regardless of whether the query results are obfuscated or not) can be automatically evaluated for patterns or common attributes within the subject records.

In some embodiments, cloud server 135 embeds a chatbot into the cloud-based application. The chatbot is configured to automatically communicate with user devices. The chatbot can communicate with a user device in a communication session, in which messages are exchanged between the user device and the chatbot. A chatbot may be configured to select answers to questions received from user devices. The chatbot may select answers from a knowledge base accessible to the cloud-based application. When a user device transmits a question to the chatbot and that chatbot does not have a pre-existing answer stored in the knowledge base, then a different representation of the question for which there is a pre-existing answer stored in the knowledge base is presented. The user communicating with the chatbot can be prompted as to whether the answer provided by the chatbot is accurate or helpful.

It will be appreciated that any machine-learning or AI algorithms may be executed to generate any of the trained machine-learning models described herein. Various types and technologies of AI-based and machine-learning models may be trained and then executed to generate one or more outputs predictive of user outcomes for performing a protocol or function. Non-limiting examples of models include Naïve Bayes models, random forest or gradient boosting models, logistic regression models, deep-learning neural networks, ensemble models, supervised learning models, unsupervised learning models, collaborative filtering models, and any other suitable machine-learning or AI models.

It will be appreciated that the cloud-based application can be configured to perform intelligent functionality with respect to consulting external physicians, determining diagnosis, and proposing treatment for any disease, condition, area of study, or disorder, including, but not limited to, COVID-19; oncology, including the following cancers: lung, breast, colorectal, prostate, stomach, liver, cervix uteri (cervical), esophagus, bladder, kidney, pancreas, endometrium, oral, thyroid, brain, ovary, skin, and gall bladder; solid tumors, such as sarcomas and carcinomas; cancers of the immune system, including lymphomas (such as Hodgkin's or non-Hodgkin's); and cancers of the blood (hematological cancers) and bone marrow, such as leukemias (such as acute lymphocytic leukemia (ALL) and acute myeloid leukemia (AML)), lymphomas, and myeloma. Additional disorders include blood disorders such as anemia; bleeding disorders such as hemophilia; blood clots; ophthalmology disorders, including diabetic retinopathy, glaucoma, and macular degeneration; neurological disorders, including multiple sclerosis, Parkinson's, disease, spinal muscular atrophy, Huntington's Disease, amyotrophic lateral sclerosis (ALS), and Alzheimer's disease; and autoimmune disorders, including multiple sclerosis, diabetes, systemic lupus erythematosus, myasthenia gravis, inflammatory bowel disease (IBD), psoriasis, Guillain-Barre syndrome, chronic inflammatory demyelinating polyneuropathy (CIDP), Graves' disease, Hashimoto's thyroiditis, eczema, vasculitis, allergies, and asthma.

Other diseases and disorders include, but are not limited to, kidney disease; liver disease; heart disease; strokes; gastrointestinal disorders such as celiac disease, Crohn's disease, diverticular disease, irritable bowel syndrome (IBS), gastroesophageal reflux disease (GERD), and peptic ulcer; arthritis; sexually transmitted diseases; high blood pressure; bacterial and viral infections; parasitic infections; connective tissue diseases; celiac disease; osteoporosis; diabetes; lupus; diseases of the central and peripheral nervous systems, such as attention deficit/hyperactivity disorder (ADHD), catalepsy, encephalitis, epilepsy, and seizures; peripheral neuropathy; meningitis; migraine; myelopathy; autism; bipolar disorder; and depression.

IV.A. The Cloud-Based Application Enables User Devices to Broadcast Consult Requests to Other User Devices and Automatically Condenses Subject Records to Comply with Data-Privacy Rules

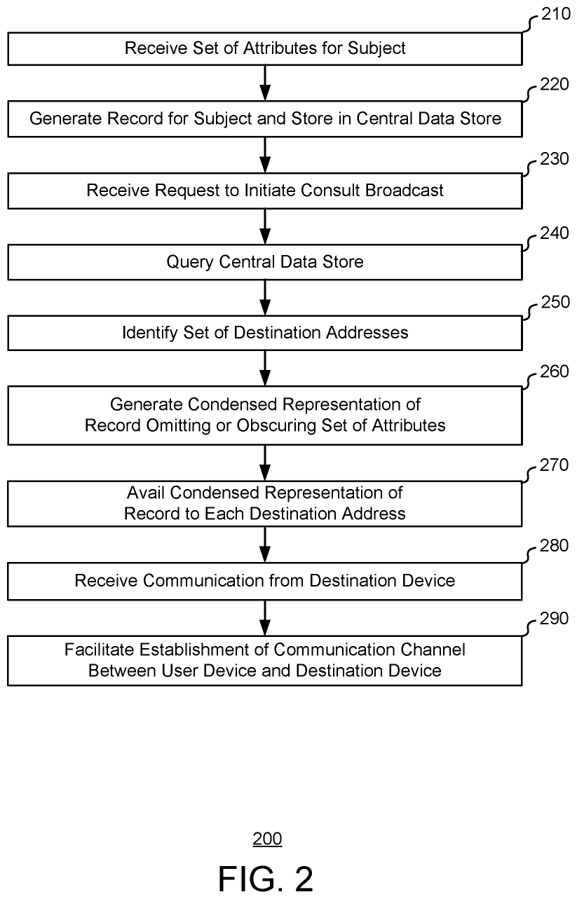

FIG. 2 is a flowchart illustrating process 200 performed by the cloud-based application to distribute condensed subject records to user devices in association with a consult broadcast requesting assistance with treating a subject. Process 200 may be performed by cloud server 135 to enable user devices associated with different entities (e.g., hospitals) to collaborate or consult regarding treatment for a subject, while complying with data-privacy rules.

Process 200 begins at block 210 where cloud server 135 receives a set of attributes from a user device. Each attribute of the set of attributes can represent any characteristic(s) of a subject (e.g., a patient). The set of attributes may be identified by a user using an interface provided by cloud server 135. For example, the set of attributes identifies demographic information of the subject and a recent symptom experienced by the subject. Non-limiting examples of demographic information include age, sex, ethnicity, state or city of residence, income range, education level, or any other suitable information. Non-limiting examples of a recent symptom include a subject who has currently or recently (e.g., at a last visit, at intake, within 24 hours, within a week) experienced a particular symptom (e.g., difficulty breathing, fever above a threshold temperature, blood pressures above a threshold blood pressure).

At block 220, cloud server 135 generates a record for the subject. The record may be a data element including one or more data fields. The record indicates each of the set of attributes associated with the subject. The record may be stored at a central data store, such as data registry 140 or any other cloud-based database. At block 230, cloud server 135 receives a request that was submitted by a user using the interface. The request may be to initiate a consult broadcast. For example, the user associated with an entity is a physician at a medical center treating a subject. The user can operate a user device to access the cloud-based application to broadcast a request for assistance with treating the subject. The broadcast may be transmitted to a set of other user devices associated with a different entity.

At block 240, cloud server 135 queries the central data store using the one or more recent symptoms included in the set of attributes associated with a subject. The query results include a set of other records. Each record of the set of other records is associated with another subject. In some instances, cloud server 135 may query the central data store to identify other subject records that are similar to the subject record. Similarity may be determined by comparing the transformed representation of the entire subject record to the transformed representation of each other subject record. The comparison of the transformed representations may result in a distance (e.g., a Euclidean distance) that represents a degree of similarity between the two subject records. In other instances, similarity may be determined based on values included in a data element. For example, a target subject record may include a target data element including text that represents symptoms experienced by a subject. Each other subject record stored in the central data store may also include a data element including text that represents the symptoms of the associated subject. Cloud server 135 can transform the text included in the target data element into a numerical representation using techniques described above (e.g., a trained convolution neural network, a text vectorization technique such as Word2Vec). The numerical representation of the text included in the target data element may be compared against the numerical representation of the text included in the matching data element of each other subject record. The result of the comparison (e.g., in a domain space, such as a Euclidean space) between two numerical representations may indicate a degree to which the text included in the target data element is similar to the text included in the data element of another subject record. At block 250, cloud server 135 identifies a set of destination addresses (e.g., other user devices associated with a different entity). Each destination address of the set of destination addresses is associated with a care provider for another subject associated with one or more other records of the set of other records identified at block 240. At block 260, cloud server 135 generates a condensed representation of the record for the subject. The condensed representation of the record omits, obscures, or obfuscates at least a portion of the record. The condensed representation of the record can be exchanged between external systems without violating data-privacy rules because the condensed representation of the record cannot be used to uniquely identify the subject associated with the record. Cloud server 135 can execute any masking or obfuscation techniques to generate the condensed representation of the record.

At block 270, cloud server 135 avails the condensed representation of the record with a connection input component (e.g., a selectable link, such as a hyperlink, that causes a communication channel to be established) to each destination address of the set of destination addresses. The connection input component may be a selectable element presented to each destination address. Non-limiting examples of the connection input component include a button, a link, an input element, and other suitable selectable elements. At block 280, cloud server 135 receives a communication from a destination device associated with a destination address. The communication includes an indication that the user operating the destination device selected the connection input component associated with the condensed representation of the record. At block 290, cloud server 135 establishes a communication channel between the user device and the destination device at which the connection input component was selected. The communication channel enables the user operating the user device (e.g., the physician treating the subject) to exchange messages or other data (e.g., a video feed) with the destination device associated with the destination address at which the connection input component was selected (e.g., a physician at another hospital who agreed to assist with the treatment of the patient).

In some embodiments, cloud server 135 is configured to automatically determine a location of the user device and a location of the destination device at which the connection input component was selected. Cloud server 135 can also compare the locations to determine whether to generate the condensed representation of the record. For example, at block 260, cloud server 135 may generate the condensed representation of the record because cloud server 135 determines that each destination address of the set of destination addresses is not collocated with the user device that initiated the consult broadcast. In this case, cloud server 135 may automatically determine to generate the condensed representation of the record to comply with data-privacy rules. As another example, if the set of destination addresses is associated with the same entity as the user device that initiated the consult broadcast, then cloud server 135 can transmit the record in full (e.g., without obfuscating a portion of the record) to a destination device associated with a destination address while still complying with the data-privacy rules.

In some embodiments, cloud server 135 generates a plurality of other condensed record representations. Each of the plurality of other condensed record representations is associated with another subject. Cloud server 135 transmits the plurality of other condensed record representations to the user device and receives, from the user device, a communication identifying selections of a subset of the plurality of other condensed record representations. Each of the set of destination addresses is represented by one of the condensed record representations. For example, generating a condensed record representation includes determining a jurisdiction of another subject associated with the condensed record representation, determining a data-privacy rule governing the exchange of subject records within the jurisdiction, and generating the condensed record representation to comply with the data-privacy rule. A first other condensed record representation of the plurality of other condensed record representations may include data of a particular type. A second other condensed record representation of the plurality of other condensed record representations may omit or obscure data of the particular type. For example, data of the particular type may be contact information, identifying information such as name and Social Security number, and other suitable information that can be used to uniquely identify the other subject.

In some implementations, a communication may be received at the central data store. The communication may be transmitted by a user device operated by a user and may include an identifier of a target subject record of a target subject. The communication, when received at the central data store, may cause the central data store to query the stored set of subject records to identify an incomplete subset of the set of subject records. Each subject record of the incomplete subset may be identified and included in the incomplete subset because the subject record is determined to be similar to the target subject record along at least one dimension. Similarity between two subject records along a dimension may represent similarity with respect to a data element of the subject records, such as similarity with respect to symptoms, diagnoses, treatments, or any other suitable data elements. The one or more dimensions, along which similarity or dissimilarity is determined, may be defined automatically or may be user defined. Determining a similarity or dissimilarity between the target subject record and each subject record of the set of subject records stored in the central data store may include at least the following operations: retrieving the target subject record based on the identifier included in the communication, generating a transformed representation of the target subject record (or retrieving the existing transformed representation of the target subject record), and performing a clustering operation using the transformed representation of the target subject record and the transformed representation of each subject record of the set of subject records. The clustering operation may be performed with respect to one or more dimensions (e.g., one or more features of a subject record). For example, the clustering operation may cluster the set of subject records stored in the central data store based on the data element that contains values representing a subject's symptoms. The transformed representation of the target subject record may include a vector representation of the data element that contains values representing the subject's symptoms. The vector representation of this data element of the target subject record and the vector representations of the corresponding data element in each subject record of the set of subject records may be compared to define clusters of subject records. Each cluster of subject records may define a group of one or more subject records that share a common characteristic associated with the data element selected as the dimension of similarity. In each cluster of subject records, a Euclidean distance may be computed between the transformed representation of the target subject record and the other transformed representations of the set of subject records. A subject record may be determined to be similar to the target subject record when, for example, the Euclidean distance between the transformed representation of the subject record and the transformed representation of the target subject record is within a threshold value.

IV.B. Updating Shareable Treatment-Plan Definitions Based on Aggregated User Integration

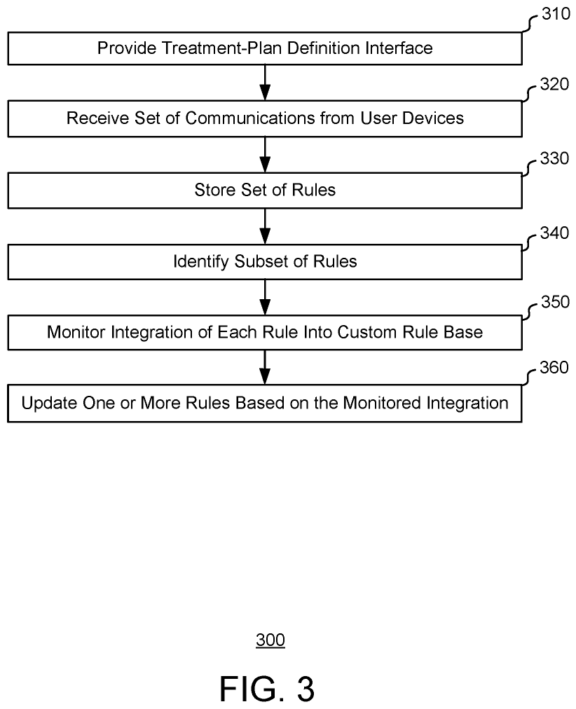

FIG. 3 is a flowchart illustrating process 300 for monitoring the user integration of treatment-plan definitions (e.g., decision trees or treatment workflows) and automatically updating the treatment-plan definitions based on a result of the monitoring. Process 300 may be performed by cloud server 135 to enable a user device to define a treatment plan for treating a population of subjects with a condition. The user device may distribute the treatment-plan definition to user devices connected to internal or external networks. The user devices receiving the treatment-plan definition can determine whether to integrate the treatment-plan definition into a custom rule base. The integration into the custom rule base can be monitored and used to automatically modify the treatment-plan definition.

At block 310, cloud server 135 stores interface data that causes a treatment-plan definition interface to be displayed when a user device loads the interface data. The treatment-plan definition interface is provided to each user device of a set of user devices when the user devices accesses cloud server 135 to navigate to the treatment-plan definition interface. In some embodiments, the treatment-plan definition interface enables a user to define a treatment plan for treating a population of subjects that have a condition (e.g., lymphoma).

At block 320, cloud server 135 receives a set of communications. Each communication of the set of communications is received from a user device of the set of user devices and was generated in response to an interaction between the user device and the treatment-plan definition interface. In some embodiments, the communication includes one or more criteria, for example, for defining a population of subject records. Each criteria may be represented by a variable type. For example, variable type may be a value or variable used as the condition of a criterion. The variable type of a criterion of a rule may also be any value of a condition that constrains the population of subjects to an incomplete sub-group. For example, the variable type of a rule that defines a population of pregnant women is “IF ‘subject is pregnant.’” A criterion may be a filter condition for filtering a pool of subject records. For example, a criterion for defining a population of subject records associated with subjects who may develop a lymphoma may include a filter condition of “abnormality in ALK” AND “over 60 years old.” The communication may also include a particular type of treatment for the condition. The particular type of treatment may be associated with performing a certain action (e.g., undergo surgery) or refraining from a certain action (e.g., reduce salt intake) that is proposed to treat the condition associated with the subjects represented by the population of subject records.

At block 330, cloud server 135 stores a set of rules in a central data store, such as data registry 140 or any other centralized server within cloud network 130. Each rule of the set of rules includes the one or more criteria and the particular treatment type included in the communication from a user device. As an illustrative example, a rule represents a treatment workflow for treating lymphoma in a subject. The rule includes the following criteria (e.g., the conditions following the “IF” statement) and a next action (e.g., the particular treatment type defined or selected by the user, and which follows the “THEN” statement): “IF ‘biopsy of lymph nodes indicates lymphoma cells are present’ AND ‘blood test reveals lymphoma cells present’ THEN ‘treat with chemotherapy’ AND ‘active surveillance.’” Additionally, each rule of the set of rules is stored in association with an identifier corresponding to the user device from which the communication was received.

At block 340, cloud server 135 identifies a subset of the set of rules that are available across entities via the treatment-plan definition interface. A subset of rules may include the subset of the set of rules associated with a condition and that are distributed to external systems, such as other medical centers, for evaluation. For example, a rule can be selected for including in the subset of rules by evaluating a characteristic of the rule or the identifier associated with the rule. The characteristic of the rule can include a code or flag stored or appended to the stored rule. The code or flag indicates the rule is generally available to external systems (e.g., availed to entities).

At block 350, for each rule of the subset of rules identified at block 340, cloud server 135 monitors interactions with the rule. An interaction may include an external entity (e.g., external to the entity associated with the user who defined the treatment plan associated with the rule) integrating the rule into a custom rule base. For example, a user device associated with an external entity (e.g., a different hospital) evaluates the rule availed to the external entity. The evaluation includes determining whether the rule is suitable for integrating into a rule set defined by the external entity. The rule may be suitable when the user device associated with the external entity indicates that the treatment workflow that is defined using the rule is suitable to treat the condition corresponding to the rule. Continuing with the illustrative example above, the rule for treating lymphoma may be availed to an external medical center. A user associated with the external medical center determines that the rule for treating lymphoma is suitable for integrating into the rule set defined by the external medical center. Thus, after the rule is integrated into a custom rule base defined by the external medical center, other users associated with the external medical center will be able to execute the integrated rule by selecting the integrated rule from the custom rule base. Additionally, cloud server 135 monitors integration of the availed rule by detecting a signal generated or caused to be generated when the treatment-plan definition interface receives input corresponding to an integration of the rule into the custom rule base from the user device associated with the external entity.

As another illustrative example, the user device associated with the external entity uses the treatment-plan definition to integrate an interaction-specified modified version of the rule into the custom rule base. The interaction-specified modified version of the rule is a portion of the rule selected for integration into the custom rule base. Selecting a portion of the rule for integration includes selecting less than all criteria included in the rule for integration into the custom rule base. Continuing with the illustrative example above, the user device associated with the external entity selects the criteria of “IF ‘biopsy of lymph nodes indicates lymphoma cells are present’” for integration into the custom rule base, but the user device does not select the criteria of “blood test reveals lymphoma cells present” for integration into the custom rule base. Thus, the interaction-specific modified version of the rule integrated into the custom rule base is “IF ‘biopsy of lymph nodes indicates lymphoma cells are present’ THEN ‘treat with chemotherapy’ AND ‘active surveillance.’” The criterion of “blood test reveals lymphoma cells present” is removed from the rule to create the interaction-specified modified version of the rule, which is integrated into the custom rule base.

At block 360, cloud server 135 may detect that the interaction-specified modified version of the rule was integrated into the custom rule base defined by the external entity. Once detected, cloud server 135 may update the rule stored at the central data store of cloud network 130. The rule may be updated based on the monitored interaction(s). The term “based on” in this example corresponds to “after evaluating” or “using a result of an evaluation of” the monitored interaction(s). For example, cloud server 135 detects that the user device associated with the external entity integrated the interaction-specified modified version of the rule. In response to detecting the interaction-specified modified version of the rule, cloud server 135 may update the rule stored in the central data store from the existing rule to the interaction-specified modified version of the rule.

In some embodiments, cloud server 135 updates the rule by generating an updated version that is to be availed across external entities. Another original version may remain un-updated and is availed to a user associated with the user device from which the one or more communications that identified the criteria and particular type of treatment were received. For example, cloud server 135 updates the rule stored at the central data store, but cloud server 135 does not update another rule of the set of rules stored at the central data store.

In some embodiments, cloud server 135 may update the rule when an update condition has been satisfied. An update condition may be a threshold value. For example, the threshold value may be a number or percentage of external entities that have integrated a modified version of the rule into their custom rule bases. As another example, the update condition may be determined using an output of a trained machine-learning model. To illustrate, cloud server 135 may input the detected signals received from external entities into a multi-armed bandit model that automatically determines whether and/or when to avail the rule and/or whether and when to avail an updated version of the rule. To illustrate and only as a non-limiting example, a rule may be defined as executable code, such that the rule upon execution automatically queries the central data store to identify a subset of the set of subject records to further analyze. Additionally, the rule may include one or more treatment protocols for treating the subjects associated with the identified subset of subject records. The rule may be defined as a workflow for defining a subset of the set of subject records and treating the subset associated with the subset of subject records. For example, the rule may include one or more criteria for filtering subject records out of the set of subject records, and for performing certain treatment protocols on the subjects associated with the remaining subject records (e.g., the subject records remaining after the filtering has been performed on the set of subject records). While the rule is defined by a user of a first entity, the rule may be accepted (e.g., integrated into a rule base of the second entity), modified, or entirely rejected by an external user (e.g., a doctor who works at a different hospital) of a second entity (e.g., the first and second entities being two different medical facilities). In some examples, each time an external user of the second entity accepts the rule and thus fully integrates the rule into its codebase, then a feedback signal may be transmitted to the cloud server 135. In other examples, each time a user of the second entity modifies the rule, then a feedback signal may be transmitted to the cloud server 135. In other examples, each time a user of the second entity entirely rejects the rule, then a feedback signal may be transmitted to the cloud server 135. In each example above, the feedback signal may include data indicating the rule (e.g., a rule identifier) and whether the rule was accepted, modified, or rejected. A multi-armed bandit model (executable by cloud server 135) can be configured to intelligently select one of the original rule, the modified rule, or an entirely different rule for broadcasting to external users of other entities. The selection of the original rule, the modified rule, or the different rule may be based at least in part on the configuration of the multi-armed bandit. In some examples, the multi-armed bandit may be configured with an epsilon greedy search technique. In an epsilon greedy search technique, the multi-armed bandit model may select the original rule for broadcasting to external users of other entities with a probability of “1−epsilon,” where epsilon represents a probability of exploring a new or modified rule. Thus, the multi-armed bandit model may select a modified version of the original rule or a completely new rule with a probability of the defined epsilon. The multi-armed bandit model may change the epsilon based on the feedback signals received from the other entities. For example, if the feedback signals indicate that the rule has been modified in a specific manner by different external users over a threshold number of times, then the multi-armed bandit model may learn to select the rule, as modified in the specific manner, to broadcast to external users, instead of broadcasting the original rule.

In some embodiments, cloud server 135 identifies multiple rules of the set of rules that include criteria corresponding to the same variable type and that identify same or similar types of treatment. A variable type may be a value or variable used as the condition of a criterion. The variable type of a criterion of a rule may also be any value of a condition that constrains the population of subjects to a sub-group. For example, the variable type of a rule that defines a population of pregnant women is “IF ‘subject is pregnant.’” Cloud server 135 determines a new rule that is a condensed representation of the multiple rules when the new rule is generally transmitted to the servers operated by other entities.

In some embodiments, cloud server 135 provides another interface configured to receive a set of attributes of a subject, for example, a user operating a user device to access the other interface and select a subject record that includes a set of attributes using the other interface. The selection of the subject record may cause cloud server 135 to receive the set of attributes of the subject. Cloud server 135 identifies (e.g., determines) a particular rule for which the criteria are satisfied based on the set of attributes of the subject. For example, the cloud server 135 evaluates the set of attributes of the subject record against the criteria of the rules stored in the central data store. To illustrate, if the set of attributes includes a data field containing the value “pregnant,” and if a rule includes a single criteria of “IF ‘subject is pregnant,” then cloud server 135 identifies this rule. Cloud server 135 updates the other interface to present the particular rule and each particular type of treatment associated with the particular rule.

In some embodiments, a criterion of a rule is a variable type that relates to a particular demographic variable and/or a particular symptom-type variable. Non-limiting examples of a demographic variable include any item of information that characterizes a demographic of the subject, such as age, sex, ethnicity, race, income level, education level, location, and other suitable items of demographic information. Non-limiting examples of a symptom-type variable indicate whether a subject currently or recently (e.g., at a last visit, at intake, within 24 hours, within a week) experienced a particular symptom (e.g., difficulty breathing, fainting, fever above a threshold temperature, blood pressures above a threshold blood pressure).

In some embodiments, cloud server 135 monitors data in a registry of subject records, such as the subject records stored in data registry 140. Cloud server 135 monitors the data in the registry of subject records for each rule of the subset of rules (identified at block 340). Cloud server 135 identifies a set of subjects for which the criteria of the rule were satisfied and for which the particular treatment was previously prescribed to the subject. Cloud server 135 identifies, for each of the set of subjects, a reported state of the subject as indicated from or using assessment or testing. For example, the reported state is any information characterizing a state of the subject in an aspect, such as whether the subject has been discharged, whether the subject is alive, measurements of the subject's blood pressure, the number of times the subject wakes up during a sleep stage, and other suitable states. Cloud server 135 determines an estimated responsiveness metric of the set of subjects to the particular treatment based on the reported states. For example, if the particular treatment of a rule is to prescribe a medication, the estimated responsiveness metric is a representation of the extent to which the medication addressed a symptom or condition experienced by the subject. As a non-limiting example, the estimated responsiveness metric of the set of subjects may be an average, a weighted average, or any summation of a score assigned to each subject of the set of subjects. The score can represent or measure the effectiveness of the subject's responsiveness to the treatment. In some instances, cloud server 135 may generate the score that represents the effectiveness of the subject's responsiveness to the treatment by using a clustering technique. To illustrate and as only a non-limiting example, a set of subject records may represent subjects who previously underwent a particular treatment protocol for treating a condition. Each subject record of the set of subject records may be labeled (e.g., by a user) as having one of a positive responsiveness to the particular treatment protocol, a neutral responsiveness to the particular treatment protocol, or a negative responsiveness to the particular treatment protocol. The set of subject records may then be divided into three subsets (e.g., clusters): a first subset of subject records may correspond to subjects who had a positive responsiveness to the particular treatment protocol, a second subset of subject records may correspond to subjects who had a neutral responsiveness to the particular treatment protocol, and a third subset of subject records may correspond to subjects who had a neutral responsiveness to the particular treatment protocol. Cloud server 135 may transform each subject record of the first subset of subject records into a transformed representation, according to implementations described above. Cloud server 135 may also transform each subject record of the second subset of subject records into a transformed representation, using techniques described above. Lastly, cloud server 135 may transform each subject record of the third subject of subject records into a transformed representation, using the techniques described above. In some implementations, determining a predicted responsiveness of a new subject to the particular treatment protocol may include transforming the new subject record of the new subject into a new transformed representation. The new transformed representation may be compared in a domain space (e.g., a Euclidean space) with the transformed representations of each cluster or subset of subject records. If the new transformed representation is closest to a centroid of the transformed representations associated with the first subset, then the new subject is predicted to have a positive responsiveness to the particular treatment. If the new transformed representation is closest to a centroid of the transformed representations of the second subset, then the new subject is predicted to have a neutral responsiveness to the particular treatment. Lastly, if the new transformed representation is closest to a centroid of the transformed representations of the third subset, then the new subject is predicted to have a negative responsiveness to the particular treatment protocol. A centroid may be a multidimensional average of the transformed representations associated with a subset. Cloud server 135 can cause the subset of the set of rules and the estimated responsiveness metrics of the set of subjects to be displayed or otherwise presented in the treatment-plan definition interface.

IV.C. Presenting Treatment Recommendations with Associated Efficacy Using Treatments Prescribed to Similar Subjects

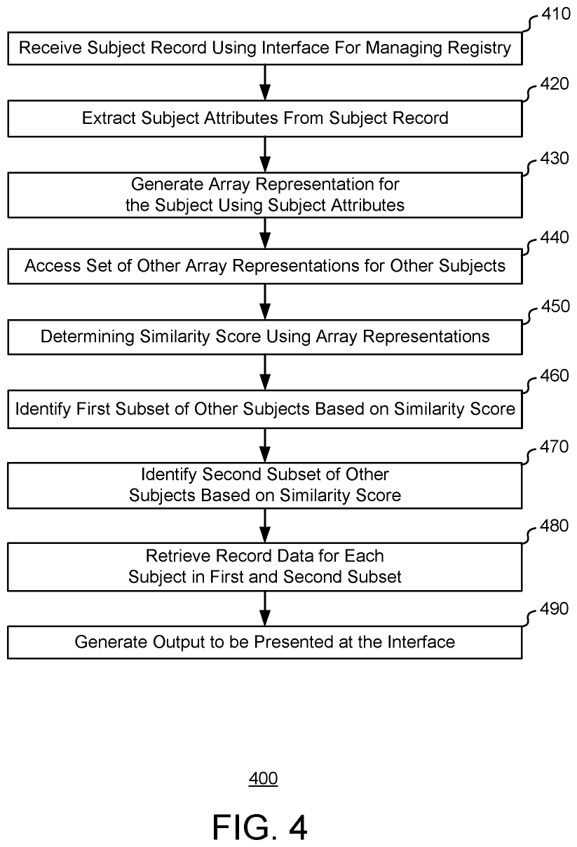

FIG. 4 is a flowchart illustrating process 400 for recommending treatments for a subject. Process 400 can be performed by cloud server 135 to display to a user device associated with a medical entity recommended treatments for a subject and the efficacy of each recommended treatment. The recommended treatments can be identified using a result of evaluating efficacies of treatments previously prescribed to similar subjects.

At block 410, cloud server 135 receives input corresponding to a subject record that characterizes aspects of a subject. The input is received from a user device associated with an entity. Further, the input is received in response to the user device selecting or otherwise identifying the subject record using an interface associated with an instance of a platform configured to manage a registry of subject records. User devices may access the interface by loading interface data stored at a web server (not shown) connected within cloud network 130. The web server may be included or executed on cloud server 135.

At block 420, cloud server 135 extracts a set of subject attributes from the subject record received at block 410. A subject attribute characterizes an aspect of the subject. Non-limiting examples of subject attributes include any information found in an electronic health record, any demographic information, an age, a sex, an ethnicity, a recent or historical symptom, a condition, a severity of the condition, and any other suitable information that characterizes the subject.

At block 430, cloud server 135 generates an array representation of the subject record using the set of subject attributes. For example, the array representation is a vector representation of the values included in the subject record. The vector representation may be a vector in a domain space, such as a Euclidean space. The array representation, however, can be any numerical representation of a value of a data field of the subject record. In some embodiments, cloud server 135 can perform feature decomposition techniques, such as SVD, to generate the values representing the set of subject attributes of the array representation of the subject record.

At block 440, cloud server 135 accesses a set of other array representations characterizing multiple other subjects. An array representation included in the set of other array representations may be a vector representation of a subject record that characterizes another subject (e.g., one of the multiple other subjects).

At block 450, cloud server 135 determines a similarity score representing a similarity between the array representation representing the subject and the array representation of each of the other subjects. For example, the similarity score is calculated using a function of a distance (in the domain space) between the array representation representing the subject and the array representation representing the other subject. To illustrate and only as a non-limiting example, the similarity score may be calculated using a range of “0” to “1,” with “0” representing a distance beyond a defined threshold and “1” representing that the array representations have no distance between them. To illustrate and only as a non-limiting example, the similarity score may be based on the Euclidean distance between two array representations (e.g., vectors).

At block 460, cloud server 135 identifies a first subset of the multiple other subjects. Subjects may be included in the first subset when the similarity score associated with a subject is within a predetermined absolute or relative range. Similarly, at block 470, cloud server 135 identifies a second subset of the multiple other subjects. However, subjects may be included in the second subset when the similarity score of this subject is within another predetermined range.

At block 480, cloud server 135 retrieves record data for each subject in the first subset and in the second subset of the multiple other subjects. The record data includes the attributes that are included in a subject record characterizing a subject. For example, the subject record data identifies a treatment received by the subject and the subject's responsiveness to the treatment. The responsiveness to the treatment may be represented by text (e.g., “subject responded positively to treatment”) or a score indicating an extent to which the subject responded positively or negatively to the treatment (e.g., a score from “0” to “1,” with “0” indicating a negative responsiveness and “1” indicating a positive responsiveness). In some instances, a treatment responsiveness may indicate a degree to which a subject responded positively to a treatment that was previously performed on the subject. For example, the treatment responsiveness may be a numerical value (e.g., a score from “0” to “10”) or non-numerical value (e.g., a word assigned to represent the responsiveness, such as “positive,” “neutral,” or “negative”). In some examples, the treatment responsiveness for previously treated subjects may be user defined. In other examples, the treatment responsiveness may be determined automatically based on a result of a test or a measurement taken from the user. For example, the treatment responsiveness may be determined automatically based on values included in a blood test performed on the subject.

At block 490, cloud server 135 generates an output to be presented at the interface on the user device. The output may indicate, for example, a recommendation of one or more treatments for the subject. The recommendation of one or more treatments may be determined based on, for example, the treatments received by the other subjects in the first and second subsets, the treatment responsiveness of subjects in the first and second subsets, and the differences between the subject attributes of subjects in the second subset and subject attributes of the subject.

In some embodiments, cloud server 135 determines that the subject and one of the subjects from the first or second subset are being treated or were treated by the same medical entities. Cloud server 135 determines that the subject and another subject of the first or second subset are being treated or were treated by different medical entities. Cloud server 135 may avail differentially obfuscated versions of records of the subjects via the interface. The cloud-based application can automatically provide differently obfuscated versions of records to entities based on varying constraints imposed on data sharing by the data-privacy rules of different jurisdictions. In some embodiments, cloud server 135 identifies the first subset and the second subset of subject records by performing a clustering operation on the transformed representations of a set of subject records.

IV.D. Automatically Obfuscating Query Results from External Entities

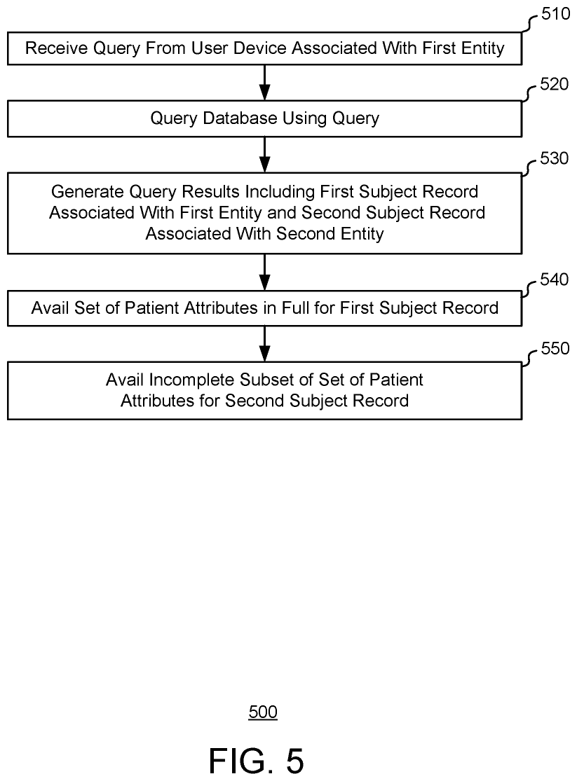

FIG. 5 is a flowchart illustrating process 500 for obfuscating query results to comply with data-privacy rules. Process 500 may be performed by cloud server 135 as an executing rule that ensures that data sharing of subject records with external entities complies with data-privacy rules. The cloud-based application may enable a user device to query data registry 140 for subject records that satisfy a query constraint. The query results, however, may include data records originating from external entities. Thus, process 500 enables cloud server 135 to provide user devices with additional information on treatments from external entities, while complying with data-privacy rules.

At block 510, cloud server 135 receives a query from a user device associated with a first entity. For example, the first entity is a medical center associated with a first set of subject records. The query may include a set of symptoms associated with a medical condition or any other information constraining a query search of data registry 140.

At block 520, cloud server 135 queries a database using the query received from the user device. At block 530, cloud server 135 generates a data set of query results that correspond to the set of symptoms and are associated with the medical conditions. For example, the user device transmits a query for subject records of subjects who have been diagnosed with lymphoma. The query results include at least one subject record from the first set of subject records (which originate or were created at the first entity) and at least one subject record from a second set of subject records associated with a second entity (e.g., a medical center different from the first entity). Each of the subject record from the first set of subject records and the subject record from the second set of subject records may include a set of subject attributes. A subject attribute can characterize any aspect of a subject.

At block 540, cloud server 135 presents (e.g., avails or otherwise makes available) to the user device the set of subject attributes in full for subject records included in the first set of subject records because these records originate from the first entity. Presenting a subject record in full includes making the set of attributes included in a subject record available to the user device for evaluation or interaction using the interface. At block 550, cloud server 135 also or alternatively avails to the user device an incomplete subset of the set of subject attributes for each subject record included in the second set of subject records. Providing an incomplete subset of the set of subject attributes provides anonymity to subjects because the incomplete subset of subject attributes cannot be used to uniquely identify a subject. For example, providing an incomplete subset may include available four of ten subject attributes to anonymize the subject associated with the ten subject attributes. In some embodiments, at block 550, cloud server 135 avails an obfuscated set of subject attributes for each subject record included in the second subject. Obfuscating the set of attributes includes reducing the granularity of information provided. For example, instead of availing the subject attribute of a subject's address, the obfuscated attribute may be a zip code or a state in which the subject lives. Whether an incomplete subject or an obfuscated subset is availed, cloud server 135 anonymizes a subject associated with the subject record.

IV.E. Chatbot Integration with Self-Learning Knowledge Base

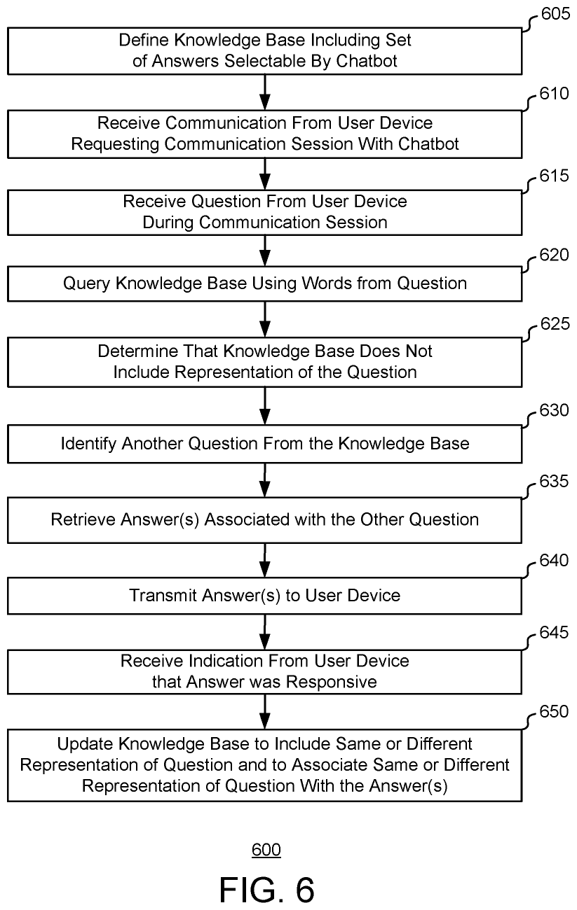

FIG. 6 is a flowchart illustrating process 600 for communicating with users using bot scripts such as a chatbot. Process 600 may be performed by cloud server 135 for automatically linking new questions provided by users to existing questions in a knowledge base to provide a response to the new question. A chatbot may be configured to provide answers to questions associated with a condition.

At block 605, cloud server 135 defines a knowledge base, which includes a set of answers. The knowledge base may be a data structure stored in memory. The data structure stores text representing the set of answers to defined questions. Each answer may be selectable by a chatbot in response to a question received from a user device during a communication session. The knowledge base may be automatically defined (e.g., by retrieving text from a data source and parsing through the text using NLP techniques) or user defined (e.g., by a researcher or physician).

At block 610, cloud server 135 receives a communication from a particular user device. The communication corresponds to a request to initiate a communication session with a particular chatbot. For example, a physician or subject may operate a user device to communicate with a chatbot in a chat session. Cloud server 135 (or a module stored within cloud server 135) may manage or establish communication sessions between user devices and chatbots. At block 615, cloud server 135 receives a particular question from the particular user device during the communication session. The question can be a string of text that is processed using NLP techniques.

At block 620, cloud server 135 queries the knowledge base using at least some words extracted from the particular question. The words may be extracted from the string of text representing the particular question using NLP techniques. At block 625, cloud server 135 determines that the knowledge base does not include a representation of the particular question. In this case, the question received may be newly posed to a chatbot. At block 630, cloud server 135 identifies another question representation from the knowledge base. Cloud server 135 may identify another question representation by comparing the question received from the user device to the other question representations stored in the knowledge base. If a similarity is determined, for example, based on an analysis of the question representations using NLP techniques, then cloud server 135 identifies the other question representation.

At block 635, cloud server 135 retrieves an answer of the set of answers associated in the knowledge base with the other question representation. At block 640, the answer retrieved at block 635 is transmitted to the particular user device as an answer to the question received, even though the knowledge base did not include a representation of the question received. At block 645, cloud server 135 receives an indication from the particular user device. For example, the indication may be received in response to the user device indicating that the answer provided by the chatbot was responsive to the particular question.

At block 650, cloud server 135 updates the knowledge base to include the representation of the particular question or different representation of the particular question. For example, storing a representation of a question includes storing keywords included in the question in a data structure. Cloud server 135 may also associate the same or different representation of the particular question with the more appropriate answer transmitted to the particular user device.

In some embodiments, cloud server 135 accesses a subject record associated with the particular user device. Cloud server 135 determines a plurality of answers to the particular question. Cloud server 135 then selects an answer from the set of answers. The selection of the answer, however, is based at least in part on one or more values included in the subject record associated with the particular user device. For example, a value included in the subject record may represent a symptom recently experienced by the subject. The chatbot may be configured to select an answer that is dependent on the symptom recently experienced by the subject. In some instances, cloud server 135 may access a learn-to-rank machine-learning model that has been trained to predict an order for each answer in a set of answers. The learn-to-rank machine-learning model may be trained using a training set of answers. Each answer of the training set of answers may be labeled with one or more symptoms and a relevance score for that symptom. The relevance score may represent a relevance of the associated answer to a given symptom of the one or more symptoms. The relevance score may be user defined or automatically determined based on certain factors, such as frequency of a word (e.g., the word(s) for the symptom) in a training answer. The training set of answers may be different from the set of answers used when the chatbot is operational in a production environment. The learn-to-rank machine-learning model may learn how to order the set of answers (used in the production environment) in terms of relevance to a symptom (which is detected from the subject profile) based on the patterns learned by the learn-to-rank model (e.g., the patterns between the labeled training set of answers and the associated relevance scores for each symptom of one or more symptoms). The chatbot may select an answer from the set of answers used in the production environment based on the predicted ordering of the set of answers. In some instances, each answer of the set of answers may be associated with a tag or code indicating one or more symptoms that are associated with the answer. Cloud server 135 may compare the value that represents the symptom recently experienced by the subject with the tag or code associated with each answer.

### V. A Network Environment Configured to Provide an Oncology Application that Facilitates Intelligent Clinical Decisions for Subjects Diagnosed with Cancer

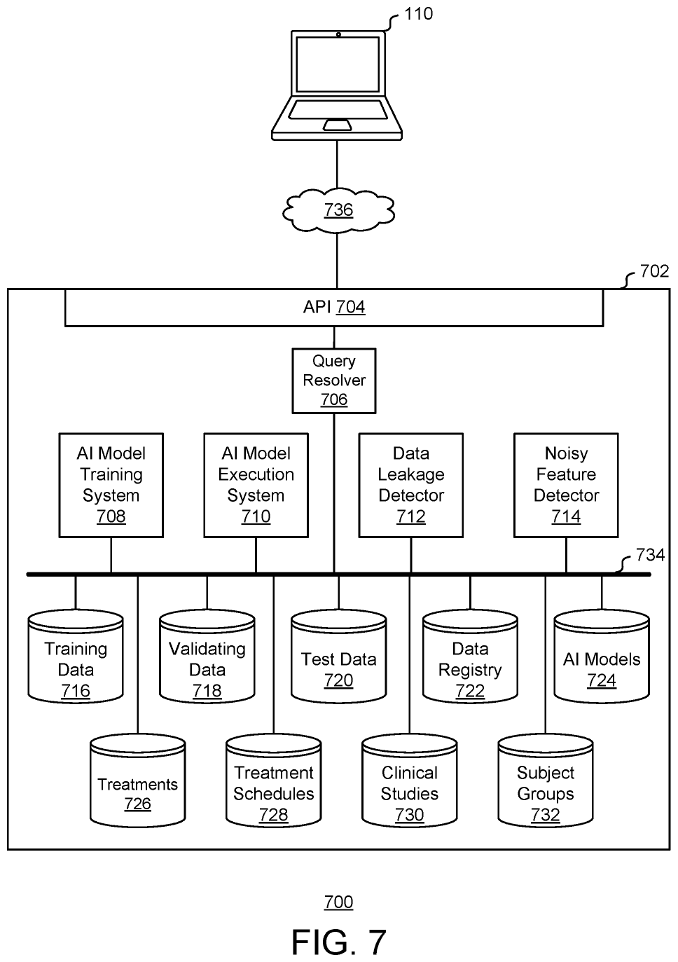

FIG. 7 is a block diagram illustrating an example of a network environment for deploying trained AI models to facilitate the subject-specific identification of treatments and treatment schedules for subjects diagnosed with cancer, according to some aspects of the present disclosure. Network environment 700 can include user device 110 and AI system 702. User device 110 can interact with AI system 702 using network 736 (e.g., any public or private network), which facilitates the exchange of communications between user device 110 and AI system 702. AI system 702 may be another implementation of AI system 145, which is described with respect to FIG. 1. User device 110 can be operated by a user, such as a physician or other medical professional who is treating a subject diagnosed with cancer. User device 110 can transmit requests to AI system 702 using application programming interface (API) 704 for triggering certain functionality (e.g., cloud-based services).

In some implementations, a physician treating a particular subject can operate user device 110 to access an oncology application (e.g., module) that is available using a cloud-based network, such as cloud network 130. The oncology application can be configured to execute certain predictive functionality that is performed using AI system 702. Non-limiting examples of predictive functionality include predicting therapeutic outcomes and subsequent cancer evolution for an individual patient based on mutation order in patients across cancer types, creating enriched patient data and predicting a progression-free survival associated with a candidate line of therapy, or automatically validating whether the reasons certain treatments on subjects were selected follow medical facility guidelines and potentially proposing new guidelines for cancer treatments based on validated treatments. While FIG. 7 illustrates a single user device 110, it will be appreciated that any number of user devices or other computing devices, such as cloud-based servers, may interact with AI system 702.

AI system 702 can perform the predictive functionality using, for example, query resolver 706, AI model training system 708, and AI model execution system 710. Query resolver 706 can include executable code that, when executed using one or more cloud-based servers of AI system 702, causes a workflow to be performed, including receiving a query from user device 110, processing the query by relaying the query to other components of AI system 702, and resolving the query by transmitting a query response to user device 110 to complete performance of the predictive functionality. A number of data structures (e.g., databases) for storing data can facilitate the predictive functionality that AI system 702 can perform. In some implementation, the data structures can store training data 716, validating data 718, test data 720, subject records from data registry 722, AI models 724, treatments 726, treatment schedules 728, clinical studies 730, and subject group identifiers 732. The various components of AI system 702 can communicate with each other using a communication network 734.

AI model training system 708 can facilitate the training of AI models using training data 716. For example, AI model training system 708 can execute code (e.g., executed by a processor, such as a physical or virtual central processing unit (CPU) of a cloud-based server), which causes training data 716 to be inputted into learning algorithms. Learning algorithms can be executed to detect patterns or correlations between data points included in training data 716. The detected patterns or correlations can be stored as an AI model, which is trained to generate an output predictive of an outcome based on the stored patterns or correlations in response to receiving an input (e.g., of new, previously unseen input data, such as a subject record for a subject not included in the training data 716).

In some implementations, as described in greater detail with respect to FIGS. 8 and 11, AI model training system 708 can facilitate the training of an unsupervised learning model that is used to cluster treatment outcomes of certain treatments. In other implementations, as described in greater detail with respect to FIGS. 9 and 12, AI model training system 708 can facilitate the training of a knowledge graph (or knowledge model) that is used to predict the progression-free survival of a particular treatment for a particular subject with a specific cancer type. In other implementations, as described in greater detail with respect to FIGS. 10 and 13, AI model training system 708 can facilitate the training of a neural network model that automatically classifies the reasons that contributed to the selection of a proposed or predicted treatment as compliant with guidelines or not compliant with guidelines.

The learning algorithms executed by AI system 702 may include any supervised, unsupervised, semi-supervised, reinforcement, and/or ensemble learning algorithms. Non-limiting examples of learning algorithms that can be executed by AI system 702 are included in Table 1 below. The selection of a learning algorithm by AI system 702 for training an AI model can be based on, for example, the type and size of at least a portion of training data 716 and the target predictive outcomes intended for the predictive functionality that AI system 702 can perform. The various learning algorithms provided in Table 1 can be used as a learning algorithm for training any of the AI-based models described herein.

TABLE 1

| Model Type | Learning Algorithm |
|---|---|
| Text Analysis | N-Gram Extraction |
| | Word-to-Vector |
| | Preprocessing Text |
| | Feature Hashing |
| Regression Analysis | Neural Network Regression |
| | Decision Tree Regression |
| | Boosted Decision Tree |
| | Fast Forest Quantile |
| | Poisson Regression |
| | Linear Regression |
| | Bayesian Linear Regression |
| Image Classification | Convolutional Neural Network |
| | Generative Adversarial Network |
| | DenseNet |
| Clustering | K-Means |
| | Mean-Shift |
| | Density-Based Spatial Clustering of |
| | Applications With Noise |
| | Expectation-Maximization (EM) Clustering |
| | Using Gaussian Mixture Models (GMM) |
| | Agglomerative Hierarchical Clustering |
| Multiclass Classification | Multiclass Logistic Regression |
| | Multiclass Boosted Decision Tree |
| | Multiclass Decision Forest |
| | Multiclass Neural Network |
| | One-vs-All Multiclass |
| Recommendation Models | Two-Class Support Vector Machine |
| | Two-Class Averaged Perceptron |
| | Two-Class Decision Forest |
| | Two-Class Logistic Regression |
| Anomaly Detection | Principal Component Analysis (PCA)-Based |
| | Anomaly Detection |
| | Support Vector Machine |

In addition, during the process of training the various AI models, AI model training system 708 can interact with training data 716, validating data 718, and test data 720. Training data 716 is the data set that is inputted into the learning algorithm. The learning algorithm detects patterns, correlations, or relationships between data points within training data 716. However, the patterns, correlations, or relationships (e.g., the parameters) detected by the learning algorithm can overfit training data 716. Overfitting occurs when the analysis executed by the learning algorithm (e.g., which generated the patterns, correlations, or relationships) corresponds exactly or substantially exactly to training data 716. In this case, the analysis executed by the learning algorithms may not accurately serve as the basis of predicting new, previously unseen input data. Therefore, validating data 718 is a different data set from training data 716 and is used to modify the patterns, correlations, or relationships to prevent overfitting the training data 716. In cases where multiple learning algorithms are executed on training data 716, validating data 718 can be used to identify the learning algorithm with the highest performance on new input data (e.g., input data that is not included in training data 716). Validating data 718 can be used to generate an error function that can be evaluated to determine the performance of each learning algorithm on new input data. For example, the patterns, correlations, or relationships detected within training data 716 by each of the various learning algorithms can be stored in various AI models. The error function of each AI model on new input data can be evaluated using validating data 718. The AI model with the lowest error function can be selected. Lastly, test data 720 is another data set which is independent from each of training data 716 and validating data 718. Test data 720 can be inputted into the selected AI model to test the overall performance of the selected AI model.

In some implementations, training data 716, validating data 718, and test data 720 can be segments across a single larger data set. For example, a data set can be segmented into three data subsets. The training data 716 can be one of the three data subsets, validating data 718 can be another one of the three data subsets, and test data 720 can be the last of the three data subsets. In some implementations, the data set that is segmented into three or more subsets can include any data or data type. Non-limiting examples of data or data types that can be included in the data set from which training data 716, validating data 718, and/or test data 720 are generated include radiological image data, MRI data, genomic profile data, clinical data (e.g., measurements, treatments, treatment responses, diagnoses, severity, medical history), subject-generated data (e.g., notes inputted by a subject with breast cancer), physician- or medical professional-generated data (e.g., physician notes), audio data representing phone recordings between a patient and a physician or other medical professional, administrative data, claims data, health surveys (e.g., Health Risk Assessment (HRS) Survey), third-party or vendor information (e.g., out-of-network lab results), public databases relevant to the subject (e.g., medical journals relevant to a subject's condition), subject demographics, immunizations, radiology reports, pathology reports, utilization information, metadata representing biological samples, social data (e.g., education level, employment status), community specifications, and so on. In some instances, at least some of the subject record can initially be identified via a communication (e.g., received at a care-provider device and/or remote server) from a device operated by the subject. In some implementations, at least some features of the subject record include or are based on one or more photographs (e.g., collected at a device of the subject or collected by a medical professional operating an imaging device). In some instances, at least some of the subject-specific data was initially identified via and/or was received from an electronic medical record corresponding to the subject.

AI model execution system 710 can be implemented using executable code that when executed by a processor (e.g., a physical or virtual CPU of a cloud-based network, such as cloud network 130) executes an instance of a specific trained AI model to generate an output. The output can be predictive of certain clinical decisions relating to oncology or other specific cancers, such as breast cancer, lung cancer, colon cancer, and hematological cancer.

To illustrate and only as a non-limiting example, AI model execution system 710 receives a request from query resolver 706 (e.g., the request originated from user device 110 operated by a user, such as a physician evaluating different options of lines of therapy to perform on a particular subject). The request from user device 110 is for AI system 702 to predict a therapeutic outcome of giving alpelisib (a chemotherapy drug) to a particular subject who has breast cancer with a PIK3CA mutation. A PIK3CA mutation is involved in many types of cancer, including breast cancer, lung cancer, colon cancer, ovary cancer, brain cancer, and stomach cancer. The PIK3CA mutation produces an altered p110α subunit, allowing PI3K to signal without stopping. Unconstrained signaling, however, may cause cells to divide in an uncontrolled manner, potentially leading to cancer. The alpelisib chemotherapy treatment inhibits PI3K, which reduces the chances of tumor growth by imposing a constraint on PI3K signaling. However, alpelisib can have various side effects on a scale of severity. Query resolver 706 processes the request and identifies which trained AI model to select for performing the prediction. In response to receiving the request, AI system 702 generates a prediction of the treatment outcome of giving alpelisib to the particular subject using the selected AI model and the subject record characterizing features of the particular subject. The selected trained AI model generates an output predicting that alpelisib will have low efficacy due to a feature of the particular subject, such as a high insulin resistance also detected in the particular subject. The predictive functionality described in this example is further described with respect to FIGS. 8 and 11.

As another illustration and only as a non-limiting example, a physician evaluates whether to perform the target therapy treatment of tumor necrosis factor (TNF)-related apoptosis inducing ligand (TRAIL) on a particular user. While there is a wide range of possible side effects of varying severity of the TRAIL treatment, the TRAIL treatment is generally intended to reduce tumor growth. AI system 702 is configured to generate predictive outputs to assist the physician in determining the likely side effects of giving the particular subject the TRAIL treatment. Accordingly, user device 110, which is operated by the physician, transmits a request to AI system 702 to generate predictions of side effects that the particular subject is likely to experience in response to receiving the TRAIL treatment. AI system 702 retrieves or accesses a knowledge graph, which is a graph of nodes that represent the various relationships between treatments and side effects of those treatments. The knowledge graph includes a set of triplet statements: the treatment, the relationship to a side effect, and the side effect. Each triplet statement represents a treatment to side effect association. A learning algorithm can be executed on the entire set of triplet statements of the knowledge graph to learn the various relationships between treatments, subject features (e.g., gene mutations), and side effects. The TRAIL treatment and the subject record for the particular subject are inputted into the AI model trained using the knowledge graph. The output is that the side effects of giving the TRAIL treatment to the particular subject are predicted to be the rare negative side effect of conditions that promote tumor growth. The predictive functionality described in this example is further described with respect to FIGS. 9 and 12.

As yet another illustration and only as a non-limiting example, user device 110 transmits a request to AI system 702 to predict whether a physician's reasons for performing a treatment on a particular subject are compliant with the oncological guidelines. For example, guidelines include the NCCN Guidelines for Clinical Practice in Oncology. Before performing the treatment, the physician can receive an automated assessment of whether the physician's reasons for selecting a specific treatment are compliant with existing treatment guidelines. AI system 702 can select a neural network trained in classifying whether a list of reasons and a proposed treatment are compliant with existing oncological guidelines. The predictive functionality described in this example is further described with respect to FIGS. 10 and 13.

Certain AI models can exhibit a technical problem of memorizing a portion of training data 716 during the training process. Memorizing a portion of training data 716 can occur when the trained AI model outputs a data element included in training data 716 as is in response to receiving input data. Data leakage refers to an AI model outputting data elements as is from the training data in response to an input of new, previously unseen data. In some cases, AI models memorize training data when the AI model is overfitted to the training data. An overfitted AI model memorizes noise contained in the training data (e.g., memorizes data elements from the training data that are not relevant to the task of learning). Thus, the AI model does not generalize predictions on new, previously unseen input data when the AI model exhibits data leakage.

Data leakage can violate privacy regulations if the training data includes sensitive or private data about subjects. To illustrate and as only a non-limiting example, training data 716 includes a subject record containing a value representing that the subject (who is characterized by the subject record) has a gene mutation linked with the early onset of Alzheimer's disease. The value representing the presence of the gene mutation for Alzheimer's disease is sensitive or private data. Therefore, various privacy laws and regulations prohibit the unauthorized disclosure of the subject's sensitive or private data (e.g., the Health Insurance Portability and Accountability Act (HIPAA)). If the trained AI model is overfitted to training data 716, however, a technical challenge arises in that the trained AI model is capable of leaking (e.g., unintentionally disclosing externally or to unauthorized users) the value representing that the subject has the gene mutation for Alzheimer's disease. In some scenarios, a privacy violation may occur if an adversary user device (e.g., operated by a user who is intentionally seeking to extract sensitive information from the AI model) can transmit inputs into the trained AI model and receive the corresponding outputs generated by the AI model. For example, if an adversary user device accesses the trained AI model using a public API, then the adversary user device can transmit inputs into the trained AI model and receive the outputs generated by the trained AI model. The adversary user device can then evaluate the various outputs received from the trained AI model to infer sensitive or private data about the training data used to train the AI model. Non-limiting examples of the sensitive or private data that can be inferred include the values indicating the presence of certain genetic mutations in a particular subject; the presence or absence of a subject record in the training data; the presence or absence of a particular subject in a particular clinical study; a correlation between the phenotypes presented by a particular subject and the genetic predisposition of the particular subject to developing a particular disease, such as breast cancer; characteristics of a particular subject's genetic profile; and any other sensitive or private data.

To solve the technical challenges with respect to data leakage as described above, certain aspects and features of the present disclosure relate to configuring a data leakage detector 712 to detect and also to prevent data leakage when AI model execution system 710 executes any of the trained AI models stored in AI models data store 724. In some implementations, data leakage detector 712 can perform certain data-leakage prevention protocols on training data 716, validating data 718, test data 720, and/or AI models 724. Performing data-leakage prevention protocols on training data 716, validating data 718, test data 720, and/or AI models 724 can inhibit or prevent the leakage of sensitive data by trained AI models. Non-limiting examples of data-leakage prevention protocols performed on data include encrypting sensitive or private data contained in subject records, data sanitization, data regularization, robust statistics, adversarial training, differential privacy, federated learning, homomorphic encryption, and other suitable techniques for inhibiting or preventing the leakage of sensitive data characterizing subjects.

Referring again to FIG. 7, a subject record can include data elements that characterize a subject feature using a large number of dimensions (e.g., hundreds or thousands of feature dimensions). Certain feature dimensions in a subject record may be useful for a target task, while other feature dimensions in the subject record may represent noisy data (e.g., features that are not useful for the target task). The high-dimensionality of subject records creates a technical challenge with respect to inputting the subject records (or the numerical representations thereof) as part of the predictive functionality provided by the various AI models associated with AI system 702. Certain aspects and features of the present disclosure relate to a noisy feature detector 714, which provides a solution to the technical challenges described above. In some implementations, noisy feature detector 714 can be configured to transform high-dimensionality subject records into reduced-dimensionality subject records by classifying a subset of subject features of the set of subject features contained in a subject record as noise. For example, the noisy feature detector 714 may execute a two-class classification model that is trained to classify subject features as either predictive for a target task or as noise. It will be appreciated that noisy feature detector 714 can also be a multi-class classification model that can classify subject features of a subject record into one or more of multiple classes (e.g., noise data, useful but not predictive for target task, and useful and predictive for target task). The reduction in dimensionality of subject records improves the computational efficiency of AI system 702 by reducing the number of feature dimensions of the subject records that AI model execution system 710 processes when providing the predictive functionality. Non-limiting examples of techniques for reducing the dimensionality of subject records include reducing features based on a criterion, reducing features based on feature category, feature selection techniques, eliminating features classified as noise by a trained classifier model, and other suitable techniques.

### VI. A Network Environment Configured to Provide an Oncology Application that Predicts Therapeutic Outcomes and Cancer Evolution Using Artificial-Intelligence Techniques

A cancerous primary mutation can be preferentially associated with secondary or tertiary mutations that cause cancer to further develop in subjects. For example, certain gene mutations that are often linked to cancer may not cause cancer on their own, but rather, the existence of a mix of several preferentially associated mutations together, and which are activated in a particular order, may trigger cancerous cell growth. In certain cancers, for example, tumors may only develop when a secondary mutation is activated after a primary mutation is activated. Therefore, selecting target therapy treatments is a challenge because targeting (e.g., inhibiting) one gene mutation may activate a secondary or tertiary gene mutation, further complicating the subject's cancer. Identifying the effects of certain target therapy treatments for a given gene mutation and across different cancer types can benefit physicians.

FIG. 8 is a block diagram illustrating an example of a network environment for deploying a trained AI model to predict the treatment outcomes and cancer evolution for subjects diagnosed with cancer, according to some aspects of the present disclosure. Network environment 800 can include user device 110 and AI system 802. AI system 802 may be similar to AI system 702 illustrated in FIG. 7; however, the components of AI system 802 may differ from the components of AI system 702.

AI system 802 can be configured to identify subjects who are similar to a particular subject in terms of mutation order. AI system 802 can be configured to filter, cluster, and generate similarity measures using AI models and subject records. In some implementations, AI system 802 can be configured to train a neural network to learn how to detect similar subjects across cancer types, such that the similarity is based on patterns detected in mutational profiles of subjects. The mutational profiles, such as the mutation order indicated by a mutational profile, do not need to be exactly the same between two subjects for the subjects to be considered similar. In other implementations, AI system 802 can be configured to train a dynamic neural network to learn aspects of similarity between two or more subject records, such that the similarity is based on, for example, mutation order or other molecular characteristics indicated by a mutational profile. As only a non-limiting example, dynamic neural networks are configured with input-dependent neurons, which allows the dynamic neural network to adaptively modify to address varying inputs. In some implementations, AI system 802 can be configured to learn similarity between two or more subject records using meta-learning techniques. For instance, meta learning may involve learning to update certain parameters of meta learning model. A meta-learning model may be based on any similarity-learning techniques, such as initialization-based techniques, hallucination-based techniques, and metric learning-based techniques.

In some implementations, training the neural networks of AI system 802 to learn how to detect similar subject records based on mutation order can include creating a data set of pairs of subject records. The pairs of subject records may not have the same mutation order; however, the mutation orders between the two subject records may differ slightly in some cases and may differ greatly in other cases. In some examples, the pairs of subject records that differ slightly can be labeled as similar subject records, whereas the pairs of subject records that differ greatly in mutation order can be labeled as dissimilar subject records. The neural network can execute learning algorithms to learn the combinations and sequences of mutation orders that exist when two mutation orders are different but similar. Likewise, the neural network can execute learning algorithms to learn the combinations and sequences of mutation orders that exist when two mutation orders are different and not similar.

To illustrate and as only a non-limiting example, a particular subject has breast cancer. User device 110 can operate the cloud-based oncology application to cause the application to access the subject record 804 characterizing the particular subject. For instance, the particular subject has an ID # of 4123; a mutation order of PTEN, TP53, BRCA1, and PIK3CA; and a cancer classification of Stage I breast cancer. Subject record 806 has an ID # of 5316; a mutation order of TP53, BCL2, and BRCA2; and a cancer classification of Stage II breast cancer. Subject record 808 has an ID # of 3142; a mutation order of TP53, KRAS, and EGFR; and a cancer classification of Stage IIIA lung cancer. Subject record 810 has an ID # of 2551; a mutation order of TP53, BRCA1, KRAS, and PIK3CA; and a cancer classification of Stage 0 colon cancer. Lastly, subject record 812 has an ID # of 5456; a mutation order of PTEN, TP53, BCL10, and GSTT1; and a cancer classification of Stage IV blood cancer. The mutation orders for each of subject records 804 through 812 are summarized in Table 2 below.

TABLE 2

| Anonymous | | |
|---|---|---|
| Subject ID | Mutation Order | Cancer Type |
| 4123 | [PTEN] → [TP53] → | Breast cancer |
| | [BRCA1] → [PIK3CA] | |
| 5316 | [TP53] → [BCL2] → | Breast cancer |
| | [BRCA2] → [N/A] | |
| 3142 | [TP53] → [KRAS] → | Lung cancer |
| | [EGFR] → [N/A] | |
| 2551 | [TP53] → [BRCA1] → | Colon cancer |
| | [KRAS] → [PIK3CA] | |
| 5456 | [PTEN] → [TP53] → | Blood cancer |
| | [BCL10] → [GSTT1] | |

The treating physician is evaluating potential treatments to give to the particular subject. The physician can operate user device 110 to cause the user device 110 to generate a request (using the cloud-based oncology application) for identifying subjects across different cancer types who have similar gene mutation order. Querying or filtering subject records may not identify all similar subject records due to a slight difference in mutation order, such as intervening mutations in a chain of mutations. AI system 802 can output a prediction that subject record 804 and subject record 810 are similar in terms of mutation order. Both subject record 804 and subject record 810 share the mutation order sequence of TP53, BRCA1, and PIK3CA, although subject record 810 has an intervening mutation of KRAS.

AI system 802 can transmit a response to the request received from user device 110. The response may indicate that subject record 810 (which is anonymized) matches closely (while not exactly) to the mutation order of subject record 804. Once the similar subject based on mutation order (and potentially other factors) is identified, the physician can evaluate the treatments given to that similar subject to determine the predicted efficacy of those treatments on the particular subject.

As an advantage, AI system 802 can identify subject records that are similar to a given subject record, even when the similar subject records are associated with different cancer types. As illustrated in FIG. 8, the subject associated with subject record 810 was treated with alpelisib to target the PIK3CA gene mutation, and the treatment outcome was effective. Therefore, the physician can select alpelisib for treating the subject associated with subject record 804 because the subject also has the PIK3CA mutation in a similar mutational order as does subject record 810.

Additionally, the cancer evolution of the subject associated with subject record 810 may be informative in the prediction of the cancer evolution for the subject associated with subject record 804, even though the subjects have different types of cancer. The fact that the two subjects have similar mutation order indicates that the two subjects are likely to experience a similar cancer evolution despite the cancers being of different types.

As yet another illustration and only as a non-limiting example, the cloud-based oncology application can identify the primary mutations, secondary mutations, tertiary mutations, and so on, detected from the genomic profile of the particular subject. The cloud-based oncology application can be configured to detect other breast cancer subjects who have the same mutation order. If another breast cancer subject has the same mutation order, then the physician can assess the breast-cancer-specific treatments given to the other subject. However, it may be possible that other subjects within the same cancer type may not have the same mutation order as the subject associated with subject record 804. In this case, certain implementations of the present disclosure include continuing to search for subject records with a similar mutation order but across different cancer types.

The cloud-based oncology application can also evaluate the clinical outcomes of a given target therapy treatment performed on the other breast cancer patients with the same mutation order to predict the therapeutic outcomes of performing the treatment on the particular patient, and the likely evolution of the breast cancer mutation for that particular patient after the target therapy treatment is performed. When the oncology application cannot find other breast cancer patients with the same mutation order as the particular patient, then the oncology application can look at patients with other cancer types, such as lung cancer. For example, the oncology application can identify a group of lung cancer patients with the same mutation order as the particular patient or a group of lung cancer patients with at least the same secondary or tertiary mutation as the particular breast cancer patient. The oncology application can then assess the clinical outcomes of the given target therapy treatment performed on the identified group of lung cancer patients to predict the therapeutic outcome of the treatment on the particular breast cancer patient.

### VII. A Network Environment Configured to Predict the Specific Side Effects of Oncological Lines of Therapy Using Artificial-Intelligence Techniques

FIG. 9 is a block diagram illustrating an example of a network environment for deploying a trained AI model to predict the subject-specific side effects of oncological treatments, according to some aspects of the present disclosure. Network environment 900 can include AI system 902 and data stores 910 through 922 for storing various contextual information relating to subjects, for example, subjects being treated at a medical facility. While FIG. 9 illustrates seven data stores (e.g., data stores 910 through 922), it will be appreciated that FIG. 9 is exemplary, and thus, any number of data stores can be included in network environment 900. AI system 902 may be similar to AI system 702 illustrated in FIG. 7; however, the components of AI system 902 may differ from the components of AI system 702. The components of AI system 902 illustrated in FIG. 9 may be in addition to, in lieu of, or a part of any components of AI system 702 illustrated in FIG. 7.

In some implementations, AI system 902 can be configured to automatically predict the specific side effects that a particular subject is likely to experience in response to receiving an oncological treatment, such as target therapy. AI system 902 can include knowledge graph 904, enriched subject record generator 906, and enriched subject records data store 908.

In some implementations, knowledge graph 904 may include a graphical representation of nodes and edges that map treatments to related side effects, and it integrates the mapping into an ontology. For example, knowledge graph 904 can be trained using a large set of triplet statements. The first word or phrase of a given triplet is a treatment, such as alpelisib. The second word or phrase of the given triplet is a relationship between the treatment and a side effect, such as “30% or less exhibit this side effect.” The third word or phrase of the given triplet is the side effect. As an illustrative example, a triplet includes [alpelisib, 10%-30% of subjects, low blood count]. A triplet can be created connecting a treatment to each one of its side effects individually. In some implementations, knowledge graph 904 can be trained based on treatment side effect ontology 922. An ontology may be a set of nodes that connects treatments to their side effects. The edge connecting two nodes represents the relationship between the treatment and the side effect (e.g., the percentage of subjects who experience the side effect or a characteristic of a subject who typically experiences the side effect). Treatment side effect ontology 922 can be created using any medical journal or drug specifications.

Further, knowledge graph 904 includes a reasoning engine that is trained to generate outputs based on the relationships between treatments and side effects captured in the knowledge graph 904. In some implementations, the reasoning engine may be trained to output logical inferences based on the knowledge graph 904 and input data (e.g., a proposed treatment to be performed on a subject). The reasoning engine makes an inference of which information to extract from the knowledge graph 904 based on the interference generated by the reasoning module. The inferences may be used to evaluate the input or to recommend actions or update the rules, for example, if the proposed treatment is the target therapy of alpelisib, and if the knowledge graph 904 includes a connection between a first node representing alpelisib and a second node representing lung problems. In this example, if the subject has asthma, the reasoning engine can automatically render a logical inference that the particular subject is likely to experience lung problems.

Enriched subject record generator 906 can extract contextual information about a particular subject from data stores 910 through 920. For example, enriched subject record generator 906 can query each data store 910 through 920 using a unique subject identifier to retrieve contextual information about the subject. The contextual information retrieved for a given subject can be appended together in an enriched subject record and stored in enriched subject records data store 908. For example, the enriched subject record for a given subject may include a subject-specific data set that is more robust than the original subject record (e.g., an electronic health record). Genomic profiles data store 910 can store the various genomic profiles of subjects. Radiological images 912 can store the various images captures by or in association with the radiology department of a hospital, for example. Medical research data store 914 can include medical journals or publications that contain data points relevant to a condition associated with the subject. For example, if the original subject record includes a data element indicating that the subject was diagnosed with breast cancer, the enriched subject record generator 906 can retrieve information relating to breast cancer stages from medical research data store 914 for inclusion in the enriched subject record associated with the subject. Clinical information data store 916 can store the clinical information characterizing the subject, such as third-party lab work, emergency room visits, measurements taken from subject, and so on. Claims data 918 can include the historical health insurance information relating to the subject, such as the explanation of benefits, the costs covered by insurer versus the costs covered by the subject, the copays, and so on. Lastly, subject-provided input data store 920 stores the data received directly from interactions with the subject. For example, the subject can maintain a journal of side effects after receiving chemotherapy. The subject's notes would be stored at subject-provided input 920.

### VIII. The Cloud-Based Application is Configured to Detect the Reasons Underlying Treatment Selections and to Automatically Classify the Detected Reasons as Guideline Compliant or not

FIG. 10 is a block diagram illustrating an example of a network environment for deploying a trained reinforcement learner to select treatments, according to some aspects of the present disclosure. Network environment 1000 can include AI system 1002. AI system 1002 may be similar to AI system 702 illustrated in FIG. 7; however, the components of AI system 1002 may differ from the components of AI system 702. The components of AI system 1002 illustrated in FIG. 10 may be in addition to, in lieu of, or a part of any components of AI system 702 illustrated in FIG. 7.

There are several clinical practice guidelines in the field of oncology. Guidelines are defined by medical authorities, such as NCCN, ASCO, and others. For example, NCCN publishes guidelines for treating various cancer types. The reasons underlying the selection of a treatment often depend largely on the experience and expertise of the treating physician. Thus, determining whether the reasons for selecting or proposing a treatment are compliant with oncological treatment guidelines is a difficult and manual task. Certain implementations of the present disclosure relate to automated, AI-based techniques for verifying whether the reasons for predicting a treatment for a particular subject with cancer comply with existing guidelines.

In some implementations, AI system 1002 can be configured to include AI model execution system 1004 and treatment guidelines verification system 1006. Further, for example, AI system 1002 can be configured to generate predictive outputs, such as a predicting the treatment outcome of a given target therapy (as in FIGS. 8 and 11) and predicting the specific side effects that a particular subject will likely experience in response to a given treatment (as in FIGS. 9 and 12). AI model execution system 1004 may be similar to AI model execution system 710, in that AI model execution system 1004 can execute any AI model stored in AI model data store 724.

In some implementations, AI model execution system 1004 can be configured to detect feature importance at each instance that an AI model is executed and a prediction is generated. Feature importance refers to a category of algorithms that assign scores to input features of a predictive AI model. A score assigned to an input feature represents the importance or degree of contribution that the input feature imposed on the output of the AI model. Using the scores, AI model execution system 1004 can also generate a second output (e.g., secondary to the predictive output, such as the prediction of a treatment selection). The second output represents the one or more input features that contributed to generating the predictive output. The input features that contributed to generating the output can represent the reasons for a treatment being proposed or predicted for selection by an AI model.

As an illustrative example, a subject has the TP53 mutation and breast cancer. Inputting the subject record 1008 for the subject into a predictive AI model predicts the treatment 1010 of “target therapy proposed=reintroduce p53 using replication-defective adenovirus (Ad-p53).” While the predictive AI model predicted treatment 1010 indicating a proposed or predicted treatment for the subject, the reason for why this treatment was proposed is unclear. Therefore, according to certain implementations described herein, the AI model execution system 1004 can be configured to perform feature importance techniques to generate a second output representing the one or more input features that serve as the reason for why the treatment was proposed. Continuing with the illustrative example, the feature importance techniques are executed and detect that the Ad-p53 treatment was proposed because the particular subject had the TP53 mutation. The Ad-p53 treatment serves as a TP53 inhibitor, which can improve progression-free survival of the subject. Non-limiting examples of feature importance techniques include linear regression feature importance, logistic regression feature importance, decision tree feature importance, random forest feature importance, XGBoost feature importance, permutation feature importance, feature selection with importance, and any other suitable feature importance techniques.

In some implementations, the input of the subject record 1008 is also inputted into treatment guidelines verification system 1006. Additionally, the treatment 1010, which indicated the proposed treatment of Ad-p53 for inhibiting the TP53 mutation or replacing the wild-type p53 protein, can be inputted into treatment guidelines verification system 1006. Lastly, the features identified as contributing to the output of the predictive AI model are also inputted into treatment guidelines verification system 1006. The output of treatment guidelines verification system 1006 may be a classification of the reasons why the predicted treatment was selected into one of several categories called compliance classes. To illustrate and only as a non-limiting example, compliance classes may include “compliant with guidelines,” “not compliant with guidelines,” or “recommended to create new guidelines for treatment.” In the example above, the reason for proposing the Ad-p53 treatment (e.g., the detection of a TP53 mutation in the subject's genomic profile) can be inputted into treatment guidelines verification system 1006, which then outputs the guideline classification of “meets guideline” 1012.

In some implementations, the treatment guidelines verification system 1006 can be a neural network classifier model having been trained to classify subject records, predicted treatments, and the features that contributed to the predicted treatments as, for example, “compliant with guidelines,” “not compliant with guidelines,” or “create new guidelines.” The training data set may include a labeled data set of data records. Each record may include one or more features of a subject, the disease the subject was diagnosed with, the treatment performed on the subject, and the features that led to the treating physician's decision to perform the treatment. Further, each record may be labeled as “compliant with guidelines,” “not compliant with guidelines,” or “create new guidelines.” Supervised machine-learning algorithms may be executed on the training data set to learn the correlations in the training data. In some implementations, the treatment guidelines verification system 1012 can be a reasoning engine that generates inferences on whether the input “reasons” for selecting a cancer treatment logically reflect the existing guidelines. Further, in some examples, the compliance class of “create new guidelines” is invoked to classify a proposed treatment selection when the reasons for selecting treatment, the treatment itself, and the guidelines result in an inconclusive output.

### IX. The Cloud-Based Application can Predict a Therapeutic Outcome for a Particular Subject Using Artificial-Intelligence Techniques

FIG. 11 is a flowchart illustrating an example of a process for predicting the treatment outcomes and cancer evolution for subjects diagnosed with cancer, according to some aspects of the present disclosure. Process 1100 can be performed by any components illustrated in FIGS. 1 and 7-10. For example, process 1100 can be performed by AI system 802. Further, process 1100 can be performed to execute an AI model that generates output predictive of the therapeutic outcome of a particular treatment proposed to be performed on a particular subject.

Process 1100 begins at block 1105, where AI system 802, for example, accesses or retrieves a subject record corresponding to a particular subject (e.g., a subject being treated at a hospital). The subject record (e.g., an electronic medical record or an electronic health record) can include any number of features (e.g., data elements containing values, such as immunizations, history of medication, age, demographics) collected from or on behalf of the subject. The subject record can include a set of features that characterize aspects of the subject. For example, the subject record can include, among a multitude of other features, a feature indicating that the subject has been diagnosed with Stage I breast cancer.

In some examples, a genomic profile is associated with the subject record. For example, the subject associated with the subject record may have undergone genetic testing for various purposes, for example, to confirm a disease diagnosis or to identify the efficacy of certain treatments. A genomic profile of the particular subject may provide the results of the genetic testing. For example, the genomic profile of the particular subject can include information about specific genes (e.g., any detected genetic mutations, levels of gene expression). The genomic profile may be helpful for various purposes, such as diagnosing a disease, selecting a treatment to perform on the subject, or assessing the side effects of a proposed treatment, such as certain drugs. In some implementations, AI system 802 retrieves the genomic profile associated with the subject record accessed at block 1105. Further, AI system 802 can extract the subject's mutation order from the genomic profile. AI system 802 can also identify the type of cancer that the subject has been diagnosed with and the proposed or predicted treatment, using the genomic profile or the subject record. For example, as illustrated in FIG. 8, the mutation order represented in the subject's genomic profile may be [mutation #1=PTEN], [mutation #2=TP53], [mutation #3=BRCA1], and [mutation #4=PIK3CA].

Non-limiting examples of features that can be contained in a subject record include radiological image data, MRI data, genomic profile data, clinical data (e.g., measurements, treatments, treatment responses, diagnoses, severity, medical history), subject-generated data (e.g., notes inputted by a subject undergoing chemotherapy), physician- or medical professional-generated data (e.g., physician notes), audio data representing phone recordings between a patient and a physician or other medical professional, administrative data, claims data, health surveys (e.g., HRS Survey), third-party or vendor information (e.g., out-of-network lab results), public databases relevant to the subject (e.g., medical journals relevant to a subject's condition), subject demographics, immunizations, radiology reports, pathology reports, utilization information, metadata representing biological samples, social data (e.g., education level, employment status), community specifications, and so on.

At block 1110, AI system 802 can identify a group of other subject records (e.g., the other anonymized subject records associated with a medical facility). AI system 802 can also filter the group of subject records by the same cancer type (e.g., to form a smaller sub-group of only subject records associated with a breast cancer diagnosis). The sub-group of subject records may also be further filtered by a proposed treatment (e.g., a combination therapy treatment).

At block 1115, AI system 802 can also perform a clustering operation on the vectorized subject records included in the sub-group based on the treatment outcome of the proposed treatment. For example, the clustering operation can be any density-based technique, hierarchical-based technique, partitioning technique, or grid-based technique for clustering data points. The clustering operation can cluster the vectorized subject records of the sub-group by treatment outcome. Non-limiting examples of proposed or predicted treatments may be chemotherapy generally, specific chemotherapy drugs, radiotherapy, combination therapy, surgery, and other suitable treatment for treating cancer. Additionally, non-limiting examples of treatment outcomes can be any outcome after performing a treatment that causes a modification in a subject's condition (e.g., change in psychological condition, change in somatic condition, change in physical condition, change in social condition) that has positive or adverse effects on the health of the subject. In some implementations, the treatment outcomes can be segmented into, for example, categories, thresholds, or ranges, such as a percentage range of increase or decrease in gene expression value after a target therapy treatment is performed. The clustering operation at block 1120 results in one or more clusters of subject records for the subjects in the sub-group. The subject records included in each cluster may be associated with the same or similar treatment and treatment outcome.

At block 1120, AI system 802 can perform a mutation-order similarity determination between the particular subject record and each other record in each cluster. For example, AI system 802 can include a neural network that has been trained to learn how to detect similar subject records based on mutation order. The training data can include a data set of pairs of subject records. The pairs of subject records may not have the same mutation order; however, the mutation orders between the two subject records may differ slightly in some cases and may differ greatly in other cases. In some examples, the pairs of subject records that differ slightly can be labeled as similar subject records, whereas the pairs of subject records that differ greatly in mutation order can be labeled as dissimilar subject records. The neural network can execute learning algorithms to learn the combinations and sequences of mutation orders that exist when two mutation orders are different but similar. Likewise, the neural network can execute learning algorithms to learn the combinations and sequences of mutation orders that exist when two mutation orders are different and not similar.

At block 1125, AI system 802 can generate a similarity measure between the vector representation of the subject record characterizing the particular subject and the vector representation of each other subject record that was determined to be similar to the particular subject record at block 1120. Non-limiting examples of techniques for generating the similarity measure include a Euclidean distance, Manhattan distance, Minkowski distance, cosine similarity, Jaccard similarity, and other suitable techniques.

At decision block 1130, AI system 802 can determine whether any of the similarity measures generated at block 1125 fall with a distance range associated with a cluster. For example, if a similarity measure between the vector representation of the subject record of the particular subject and the vector representation of another subject record is within a threshold distance of a cluster, then the similarity measure may fall within the range of that cluster. When the output of decision block 1130 is “yes,” then process 1100 proceeds to block 1135, where AI system 802 uses the treatment outcome associated with the cluster (identified or selected at decision block 1130) to generate the prediction of the treatment outcome for the particular subject.

When the output of decision block 1130 is “no,” then process 1100 proceeds to block 1140. At block 1140, AI system 802 can refilter the group of other subject records by the same mutation order, but not by cancer type. Therefore, unlike the filtered sub-group formed at block 1120, the new filtered sub-group formed at block 1140 includes subject records with the same mutation order as the particular subject, but with various cancer types that may differ from the cancer type associated with the particular subject. AI system 802 can also re-perform the clustering operation on the new filtered sub-group by treatment outcome. Lastly, AI system 802 can regenerate a similarity measure between the vectorized subject record of the particular subject and each other subject record.

At decision block 1145, AI system 802 can determine whether any of the similarity measures generated at block 1140 fall with a distance range (e.g., a Euclidean distance) associated with a cluster. For example, if a similarity measure between the vector representation of the subject record of the particular subject is within a threshold distance of a cluster, then the similarity measure may fall within the range of that cluster. When the output of decision block 1145 is “yes,” then process 1100 proceeds to block 1150, where AI system 802 uses the treatment outcome associated with the cluster (identified or selected at decision block 1145) to generate the prediction of the treatment outcome for the particular subject. When the output of decision block 1145 is “no,” then process 1100 proceeds back to block 1140 to refilter the other subject records by a different cancer type.

### X. The Cloud-Based Application can Automatically Predict the Outcome of Mutation-Targeting Treatments for a Particular Subject

FIG. 12 is a flowchart illustrating an example of a process for predicting the subject-specific treatment outcomes of mutation-targeting treatments, according to some aspects of the present disclosure. Process 1200 can be performed by any components illustrated in FIGS. 1 and 7-10. For example, process 1200 can be performed by AI system 902. Further, process 1200 can be performed to execute AI models that generate outputs predictive of the survival advantage of proposed treatments for a subject diagnosed with cancer.

Process 1200 begins at block 1210, where AI system 902 identifies a particular subject and retrieves the subject record that characterizes the particular subject. For example, the subject record can be retrieved from a data registry, such as data registry 722. The subject records can be accessed automatically on a regular or irregular time interval or in response to a user input triggering the predictive functionality described in greater detail herein. As an illustrative example, AI system 902 can identify the particular subject based on an input received from a user device (e.g., user device 110). AI system 902 can detect a unique subject identifier (e.g., a patient code) that uniquely identifies the particular subject from the input received from the user device. AI system 902 can then query a data registry using the unique subject identifier.

At block 1220, AI system 902 (e.g., via enriched subject record generator 906) can also query other databases for contextual information characterizing the particular subject. Non-limiting examples of other databases that AI system 902 can query include genomic profile data store 910, radiological images data store 912, medical research data store 914, clinical data store 916, claims data store 918, and subject-provided input data store 920. In some examples, AI system 902 can query a genomic profile data store 910 using the unique subject identifier for results of genomic tests performed on the particular subject. To illustrate, a gene panel may have been sequenced for the particular subject, and the results of the genetic sequencing may be stored in a genomic profile at genomic profile data store 910. In some examples, AI system 902 can query claims data store 918 to retrieve health insurance claims submitted by or on behalf of the particular subject.

At block 1230, AI system 902 (e.g., via enriched subject record generator 906) can generate an enriched subject record for the particular subject. The enriched subject record for the particular subject can include the original subject record characterizing the particular subject (retrieved at block 1210) and the contextual information characterizing the particular subject (retrieved at block 1220). For example, all or part of the contextual information for the particular subject can be appended to the original subject record retrieved at block 1210. In some implementations, the enriched subject profile can include at least a part of the genomic profile of the particular subject. For example, the subject profile can include a known genetic mutation detected from a gene panel performed for the particular subject. The genomic profile of the particular subject is often stored separately or independently from the subject record characterizing the particular subject. Therefore, as a technical advantage, the enriched subject record generator 906 can store or append to the subject record at least part of the genomic profile of the particular subject. The enriched subject record can then be processed using AI system 902 to perform certain predictive functionality.

At block 1240, AI system 902 can transform the enriched subject record into a query for the knowledge model (e.g., knowledge graph 904). In some implementations, transforming the enriched subject record into a query can include transforming each data element of the enriched subject record into a numerical representation (e.g., vector), and then combining (e.g., using addition, averaging, or concatenation) the numerical representation of each data element into a single numerical representation that represents the entire enriched subject record. In some implementations, transforming the enriched subject record into a query can include generating an array of vectors, such that each element of the array represents a value of a data element of the enriched subject record. In some implementations, transforming the enriched subject model into a query may include extracting values from the enriched subject model and forming an input graph of the extracted values. The input graph may serve as an input to the knowledge model. For example, AI system 902 can extract a detected mutation from the genomic profile and a proposed treatment included in the enriched subject record. AI system 902 can transform the extracted mutation and the proposed treatment into an input graph, in which the detected mutation is a node connected to another node representing the subject's disease or health condition, which is then connected to yet another node representing the proposed treatment. The input graph can be used to query the knowledge model to predict the specific survival advantage of the proposed treatment for the particular subject.

Further, at block 1240, the input graph may or may not include the proposed treatment for treating the subject. When the input graph includes a specific proposed treatment for the particular subject, process 1200 can proceed to block 1250. At block 1250, AI system 902 can query the knowledge model using the input graph, which includes a node representing a specific proposed treatment for the subject. In response to the query, the knowledge model can generate an output representing the contextual survival advantage of the proposed treatment specifically for the particular subject. However, at block 1240, the knowledge model can also receive as input the input graph without a node representing the proposed treatment. In this situation, process 1200 proceeds to block 1270, where the knowledge model is queried using the input graph (e.g., which does not include a proposed treatment). For example, at block 1270, the knowledge model can be queried to identify the candidate treatments that are available, given the contextual information included in the enriched subject record. Further, the knowledge model can also store several potential survival advantages for each candidate treatment. Then, at block 1280, the knowledge model can also output the subject-specific survival advantage for each candidate treatment.

### XI. The Cloud-Based Application can Automatically Predict the Subject Features that Contributed to a Treatment Prediction and Determine Whether the Predicted Subject Features Comply with Hospital Guidelines

FIG. 13 is a flowchart illustrating an example of a process for deploying AI models to identify the factors (e.g., the features relating to a subject) that contributed to the prediction of a given treatment outputted by the AI system, according to some aspects of the present disclosure. Process 1300 can be performed by any components illustrated in FIGS. 1 and 7-10. For example, process 1300 can be performed by AI system 1002. Further, process 1300 can be performed to execute and automatically validate whether the subject features that contributed to a treatment prediction by the AI system comply with existing guidelines (e.g., guidelines established by a medical facility).

Process 1300 begins at block 1310, where AI system 1002 accesses or retrieves a subject record stored in the data registry, for example, data registry 722. The subject record may characterize a particular subject who has been diagnosed with cancer, such as breast cancer. At block 1320, the subject record accessed or retrieved at block 1310 can be transformed into numerical representations (e.g., vector representations) using various implementations described herein (e.g., described with respect to FIGS. 1-6). The subject records may be transformed or vectorized into numerical representations in advance or in real time or substantially real time with the performance of block 1310.

At block 1330, the numerical representation can be inputted into a trained AI model for processing, for example, using AI model execution system 710. While block 1330 can be performed using any AI model, such as the AI models described with respect to FIG. 7, for purposes of illustration, the trained AI model can output a prediction of a treatment to perform on a subject. It will be appreciated that the trained AI model executed at block 1330 can also be any of the AI models described with respect to FIGS. 12 and 13. Whichever AI model is executed in block 1330, the AI model can be trained to generate two outputs. For example, at block 1340, the AI model outputs a prediction of a treatment to perform on the particular subject, and at block 1350, the AI model also outputs the features (e.g., the data elements of the particular subject record that drove or contributed to predicting the selected treatment). As an illustrative example, a subject has Stage I breast cancer. The subject's genomic profile indicates that the subject has the PIK3CA mutation in addition to PTEN, TP53, and BRCA1. PIK3CA mutations can lead to hyperactivation of PI3Kα, a major upstream component of the PI3K pathway. The trained AI model has learned from the training data that there is a high correlation between subjects with breast cancer who have the PIK3CA mutation and subjects who are treated with alpelisib. The alpelisib treatment inhibits both the PI3K and ER pathways. Therefore, when the AI model detects that the particular subject has the PIK3CA mutation and has been diagnosed with breast cancer, the AI model generates an output selecting alpelisib as the optimal treatment for the particular subject. The trained AI model also detects that the feature of the PIK3CA mutation and the feature of the breast cancer diagnosis contributed to the prediction of alpelisib as the optimal treatment for the particular subject.

At block 1360, a treatment guidelines verification system can receive, as input, the treatment prediction (generated at block 1340) and the features predicted to have contributed to the treatment prediction (generated at block 1350). In some implementations, the treatment guidelines verification system can be a neural network classifier model having been trained to classify subject records, predicted treatments, and the features that contributed to the predicted treatments as, for example, “compliant with guidelines,” “not compliant with guidelines,” or “create new guidelines.” The training data set may include a labeled data set of data records. Each record may include one or more features of a subject, the disease the subject was diagnosed with, the treatment performed on the subject, and the features that led to the treating physician's decision to perform the treatment. Further, each record may be labeled as “compliant with guidelines,” “not compliant with guidelines,” or “create new guidelines.” Supervised machine-learning algorithms may be executed on the training data set to learn the correlations in the training data. At block 1370, once trained, the treatment guidelines verification system can classify a proposed treatment and the reasons for selecting the proposed treatment as “compliant with guidelines” (block 1372), “treatment not compliant with guidelines” (block 1374), or “create new guidelines for treatment” (block 1376).

### XII. Additional Considerations

Some embodiments of the present disclosure include a system including one or more data processors. In some embodiments, the system includes a non-transitory computer-readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions that have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

The ensuing description provides preferred exemplary embodiments only and is not intended to limit the scope, applicability, or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form not to obscure the embodiments in unnecessary detail. In other instances, well-known circuits, processes, algorithms, structures, and techniques may be shown without unnecessary detail in order to avoid obscuring the embodiments.

### XIII. Additional Examples

As used below, any reference to a series of examples is to be understood as a reference to each of those examples disjunctively (e.g., “Examples 1-4” is to be understood as “Examples 1, 2, 3, or 4”).

Example 1 is a computer-implemented method for predicting subject-specific outcomes of oncological lines of therapy, the method comprising: identifying a particular subject having been diagnosed with a type of cancer, wherein a line of therapy is proposed to be performed on the particular subject; retrieving a genomic data set corresponding to the particular subject, the genomic data set including a mutation order, and the mutation order including a series of multiple genetic mutations that mutated at different times; identifying a set of other subjects having been diagnosed with the same type of cancer as the subject, and each other subject having undergone the line of therapy and being associated with a treatment outcome; retrieving another genomic data set for each other subject of the set of other subjects, the other genomic data set including another mutation order; inputting, for each other subject of the set of other subjects, the mutation order of the particular subject and the other mutation order of the other subject into a trained similarity model, the trained similarity model having been trained to generate a similarity weight representing a predicted degree to which the mutation order of the particular subject is similar to the other mutation order of the other subject; determining, based on the similarity weights outputted by the trained similarity model, a predicted treatment outcome of performing the line of therapy on the particular subject, wherein upon determining that at least one of the similarity weights outputted by the similarity model is within a threshold, identifying one of the other subjects based on the determination and assigning the treatment outcome of the identified other subject as the predicted treatment outcome for the particular subject; and/or upon determining that none of the similarity weights outputted by the similarity model are within the threshold, identifying another set of subjects having been diagnosed with a different type of cancer than the particular subject to search for a mutation order that is similar to the mutation order of the particular subject.

Example 2 is the computer-implemented method for predicting subject-specific outcomes of oncological lines of therapy, as recited in example 1, further comprising: retrieving yet another mutation order for each other subject of the other set of other subjects, each other subject of the other set having a different type of cancer than the particular subject; inputting, for each other subject of the other set of other subjects, the mutation order of the particular subject and the other mutation order of the other subject of the other set into the trained similarity model; determining, based on the similarity weights outputted by the trained similarity model, that at least one of the similarity weights outputted by the similarity model is within the threshold; and identifying one of the other subjects of the other set based on the determination and assigning of the treatment outcome of the identified other subject of the other set as the predicted treatment outcome for the particular subject.

Example 3 is the computer-implemented method for predicting subject-specific outcomes of oncological lines of therapy, as recited in examples 1-2, further comprising performing a clustering operation on a set of other subject records, the clustering operation being based on one or more outcomes of the line of therapy and forming one or more clusters.

Example 4 is the computer-implemented method for predicting subject-specific outcomes of oncological lines of therapy, as recited in examples 1-3, wherein the similarity model is trained using a training data set, wherein the training data set includes pairs of mutation orders labeled as being similar or not similar.

Example 5 is the computer-implemented method for predicting subject-specific outcomes of oncological lines of therapy, as recited in examples 1-4, wherein the predicted treatment outcome includes one or more subject-specific side effects or a progression-free survival specific to characteristics of the particular subject.

Example 6 is the computer-implemented method for predicting subject-specific outcomes of oncological lines of therapy, as recited in examples 1-5, wherein contextual information associated with the particular subject includes the genomic profile associated with the subject.

Example 7 is the computer-implemented method for predicting subject-specific outcomes of oncological lines of therapy, as recited in examples 1-6, further comprising generating the contextual information associated with the particular subject by: querying a genomic profile data store for the genomic profile associated with the particular subject; querying a radiological images data store for one or more radiological images associated with the particular subject; querying a medical research data store for content data relating to at least one feature attributed to particular the subject; querying a clinical information data store for clinical information associated with the particular subject; querying a claims data store for one or more health insurance claims submitted by or on behalf of the particular subject; and/or querying a subject-provided input data store for subject data provided by the particular subject, wherein the subject data is in one or more data formats.

Example 8 is the computer-implemented method for predicting subject-specific outcomes of oncological lines of therapy, as recited in examples 1-7, wherein the treatment outcome includes one or more subject-specific side effects, which are outputted at a computing device of the subject using a chatbot.

Example 9 is the computer-implemented method for predicting subject-specific outcomes of oncological lines of therapy, as recited in examples 1-8, wherein the subject record includes data identified in an electronic medical record corresponding to the subject.

Example 10 is the computer-implemented method for predicting subject-specific outcomes of oncological lines of therapy, as recited in examples 1-9, wherein the type of cancer with which the subject is diagnosed includes at least one or more of breast cancer, lung cancer, colon cancer, or hematological cancer.

Example 11 is the computer-implemented method for predicting subject-specific outcomes of oncological lines of therapy, as recited in examples 1-10, wherein a knowledge graph is accessible using a cloud-based oncological application configured to provide predictive functionality relating to clinical decision making.

Example 12 is the computer-implemented method for predicting subject-specific outcomes of oncological lines of therapy, as recited in examples 1-11, further comprising detecting data leakage associated with the reasoning module, the data leakage exposing a feature of the set of features included in the subject record or exposing an item of the contextual information associated with the subject; and in response to detecting data leakage associated with the reasoning module, executing a data leakage prevention protocol that prevents or blocks exposure of the feature of the set of features included in the subject record.

Example 13 is the computer-implemented method for predicting subject-specific outcomes of oncological lines of therapy, as recited in examples 1-12, further comprising generating, using a feature-selection model, a reduced-dimensionality subject record characterizing the subject, the reduced-dimensionality subject record removing one or more features from the set of features included in the subject record, the one or more features being characterized as noise.

Example 14 is a system comprising one or more processors; and a non-transitory computer-readable storage medium containing instructions which, when executed on the one or more processors, cause the one or more processors to perform part or all of one or more computer-implemented methods disclosed herein.

Example 15 is a computer-program product tangibly embodied in a non-transitory, machine-readable storage medium, including instructions configured to cause one or more data processors to perform part or all of one or more computer-implemented methods disclosed herein.

Example 16 is a computer-implemented method for predicting subject-specific side effects of oncological lines of therapy, the method comprising: accessing a knowledge graph representing an ontology for mapping side effects to lines of therapy for treating cancer; retrieving a subject record associated with a subject, the subject record including a set of features characterizing the subject, the subject having been diagnosed with a type of cancer, and the subject record including a candidate line of therapy for the subject; querying one or more data stores for contextual information that uniquely characterizes the subject; generating an enriched subject record by appending the contextual information to the subject record; transforming the enriched subject record into input data for the knowledge graph; inputting the input data into the knowledge graph; and generating, based on an output of the knowledge graph, a prediction of one or more subject-specific side effects for the candidate line of therapy, the one or more subject-specific side effects being identified based on the mapping of the side effects to the lines of therapy.

Example 17 is the computer-implemented method for predicting subject-specific side effects of oncological lines of therapy, as recited in example 16, wherein the knowledge graph is defined based on a set of triplet statements, wherein each triplet statement of the set of triplet statements includes three data elements, wherein the three data elements include: a line of therapy for treating cancer, a side effect of the line of therapy, and a relationship between the line of therapy and the side effect; and wherein the mapping of side effects to lines of therapy is based on the set of triplet statements.

Example 18 is the computer-implemented method for predicting subject-specific side effects of oncological lines of therapy, as recited in examples 16-17, wherein the knowledge graph further comprises a reasoning module configured to generate a logical inference based on the candidate line of therapy included in the input data and the mapping of side effects to lines of therapy defined by the knowledge graph.

Example 19 is the computer-implemented method for predicting subject-specific side effects of oncological lines of therapy, as recited in examples 16-18, wherein the logical inference generated by the reasoning module identifies an incomplete subset of side effects from a set of side effects included in the knowledge graph, and wherein the incomplete subset of side effects corresponding to the one or more subject-specific side effects that are predicted to occur after the candidate line of therapy is performed on the subject.

Example 20 is the computer-implemented method for predicting subject-specific side effects of oncological lines of therapy, as recited in examples 16-19, wherein the set of triplet statements that defines the knowledge graph is based on medical research, and/or wherein the one or more subject-specific side effects includes a progression-free survival specific to characteristics of the subject.

Example 21 is the computer-implemented method for predicting subject-specific side effects of oncological lines of therapy, as recited in examples 16-20, wherein the contextual information includes a genomic profile associated with the subject.

Example 22 is the computer-implemented method for predicting subject-specific side effects of oncological lines of therapy, as recited in examples 16-21, wherein the querying of the one or more data stores further comprises: querying a genomic profile data store for a genomic profile associated with the subject; querying a radiological images data store for one or more radiological images associated with the subject; querying a medical research data store for content data relating to at least one feature attributed to the subject; querying a clinical information data store for clinical information associated with the subject; querying a claims data store for one or more health insurance claims submitted by or on behalf of the subject; and/or querying a subject-provided input data store for subject data provided by the subject, wherein the subject data is in one or more data formats.

Example 23 is the computer-implemented method for predicting subject-specific side effects of oncological lines of therapy, as recited in examples 16-22, wherein the one or more subject-specific side effects are outputted at a computing device of the subject using a chatbot.

Example 24 is the computer-implemented method for predicting subject-specific side effects of oncological lines of therapy, as recited in examples 16-23, wherein the subject record includes data identified in an electronic medical record corresponding to the subject.

Example 25 is the computer-implemented method for predicting subject-specific side effects of oncological lines of therapy, as recited in examples 16-24, wherein the type of cancer with which the subject is diagnosed includes at least one or more of breast cancer, lung cancer, colon cancer, or hematological cancer.

Example 26 is the computer-implemented method for predicting subject-specific side effects of oncological lines of therapy, as recited in examples 16-25, wherein the knowledge graph is accessible using a cloud-based oncological application configured to provide predictive functionality relating to clinical decision making.

Example 27 is the computer-implemented method for predicting subject-specific side effects of oncological lines of therapy, as recited in examples 16-26, further comprising: detecting data leakage associated with the reasoning module, the data leakage exposing a feature of the set of features included in the subject record or exposing an item of the contextual information associated with the subject; and in response to detecting data leakage associated with the reasoning module, executing a data-leakage prevention protocol that prevents or blocks exposure of the feature of the set of features included in the subject record.

Example 28 is the computer-implemented method for predicting subject-specific side effects of oncological lines of therapy, as recited in examples 16-27, further comprising: generating, using a feature-selection model, a reduced-dimensionality subject record characterizing the subject, the reduced-dimensionality subject record removing one or more features from the set of features included in the subject record, the one or more features being characterized as noise.

Example 29 is a system comprising: one or more processors, and a non-transitory computer-readable storage medium containing instructions which, when executed on the one or more processors, cause the one or more processors to perform part or all of one or more computer-implemented methods disclosed herein.

Example 30 is a computer-program product tangibly embodied in a non-transitory machine-readable storage medium, including instructions configured to cause one or more data processors to perform part or all of one or more computer-implemented methods disclosed herein.

1. A computer-implemented method for predicting subject-specific outcomes of oncological lines of therapy, the method comprising:
identifying a particular subject having been diagnosed with a type of cancer, wherein a line of therapy is proposed to be performed on the particular subject;
retrieving a genomic data set corresponding to the particular subject, the genomic data set including a mutational profile indicating one or more molecular characteristics of the particular subject;
identifying a set of other subjects having been diagnosed with the same type of cancer as the subject, and each other subject having undergone the line of therapy and being associated with a treatment outcome;
retrieving another genomic data set for each other subject of the set of other subjects, the other genomic data set including another mutational profile;
inputting, for each other subject of the set of other subjects, the mutational profile of the particular subject and the other mutational profile of the other subject into a trained similarity model, the trained similarity model having been trained to generate a similarity weight representing a predicted degree to which the mutational profile of the particular subject is similar to the other mutational profile of the other subject;
determining, based on the similarity weights outputted by the trained similarity model, a predicted treatment outcome of performing the line of therapy on the particular subject, wherein:
upon determining that at least one of the similarity weights outputted by the similarity model is within a threshold, identifying one of the other subjects based on the determination and assigning the treatment outcome of the identified other subject as the predicted treatment outcome for the particular subject; and/or
upon determining that none of the similarity weights outputted by the similarity model are within the threshold, identifying another set of subjects having been diagnosed with a different type of cancer than the particular subject to search for a mutational profile that is similar to the mutational profile of the particular subject.

2. The computer-implemented method for predicting subject-specific outcomes of oncological lines of therapy, as recited in claim 1, further comprising:
retrieving yet another mutational profile for each other subject of the other set of other subjects, each other subject of the other set having a different type of cancer than the particular subject;
inputting, for each other subject of the other set of other subjects, the mutational profile of the particular subject and the other mutational profile of the other subject of the other set into the trained similarity model;
determining, based on the similarity weights outputted by the trained similarity model, that at least one of the similarity weights outputted by the similarity model is within the threshold; and
identifying one of the other subjects of the other set based on the determination and assigning the treatment outcome of the identified other subject of the other set as the predicted treatment outcome for the particular subject; and/or
wherein the mutational profile includes a mutational profile associated with the particular subject, wherein the mutation order represents a series of multiple genetic mutations that mutated at different times.

3. The computer-implemented method for predicting subject-specific outcomes of oncological lines of therapy, as recited in claim 1, further comprising:
performing a clustering operation on a set of other subject records, the clustering operation being based on one or more outcomes of the line of therapy and forming one or more clusters.

4. The computer-implemented method for predicting subject-specific outcomes of oncological lines of therapy, as recited in claim 1, wherein the similarity model is trained using a training data set, wherein the training data set includes pairs of mutational profiles labeled as being similar or not similar.

5. The computer-implemented method for predicting subject-specific outcomes of oncological lines of therapy, as recited in claim 1, wherein the predicted treatment outcome includes one or more subject-specific side effects or a progression-free survival specific to characteristics of the particular subject.

6. The computer-implemented method for predicting subject-specific outcomes of oncological lines of therapy, as recited in claim 1, wherein contextual information associated with the particular subject includes the genomic profile associated with the subject.

7. The computer-implemented method for predicting subject-specific outcomes of oncological lines of therapy, as recited in claim 1, further comprising:
generating the contextual information associated with the particular subject by:
querying a genomic profile data store for the genomic profile associated with the particular subject;
querying a radiological images data store for one or more radiological images associated with the particular subject;
querying a medical research data store for content data relating to at least one feature attributed to particular the subject;
querying a clinical information data store for clinical information associated with the particular subject;
querying a claims data store for one or more health insurance claims submitted by or on behalf of the particular subject; and/or
querying a subject-provided input data store for subject data provided by the particular subject, wherein the subject data is in one or more data formats.

8. The computer-implemented method for predicting subject-specific outcomes of oncological lines of therapy, as recited in claim 1, wherein the treatment outcome includes one or more subject-specific side effects, which are outputted at a computing device of the subject using a chatbot.

9. The computer-implemented method for predicting subject-specific outcomes of oncological lines of therapy, as recited in claim 1, wherein the subject record includes data identified in an electronic medical record corresponding to the subject.

10. The computer-implemented method for predicting subject-specific outcomes of oncological lines of therapy, as recited in claim 1, wherein the type of cancer with which the subject is diagnosed includes at least one or more of breast cancer, lung cancer, colon cancer, or hematological cancer.

11. The computer-implemented method for predicting subject-specific outcomes of oncological lines of therapy, as recited in claim 1, wherein a knowledge graph is accessible using a cloud-based oncological application configured to provide predictive functionality relating to clinical decision-making.

12. The computer-implemented method for predicting subject-specific outcomes of oncological lines of therapy, as recited in claim 1, further comprising:
detecting data leakage associated with the reasoning module, the data leakage exposing a feature of the set of features included in the subject record or exposing an item of the contextual information associated with the subject; and
in response to detecting data leakage associated with the reasoning module, executing a data-leakage prevention protocol that prevents or blocks exposure of the feature of the set of features included in the subject record.

13. The computer-implemented method for predicting subject-specific outcomes of oncological lines of therapy, as recited in claim 1, further comprising:
generating, using a feature-selection model, a reduced-dimensionality subject record characterizing the subject, the reduced-dimensionality subject record removing one or more features from the set of features included in the subject record, the one or more features being characterized as noise.

14. A system comprising:
one or more processors; and
a non-transitory computer-readable storage medium containing instructions which, when executed on the one or more processors, cause the one or more processors to perform the following operations:
identifying a particular subject having been diagnosed with a type of cancer, wherein a line of therapy is proposed to be performed on the particular subject;
retrieving a genomic data set corresponding to the particular subject, the genomic data set including a mutational profile indicating one or more molecular characteristics of the particular subject;
identifying a set of other subjects having been diagnosed with the same type of cancer as the subject, and each other subject having undergone the line of therapy and being associated with a treatment outcome;
retrieving another genomic data set for each other subject of the set of other subjects, the other genomic data set including another mutational profile;
inputting, for each other subject of the set of other subjects, the mutational profile of the particular subject and the other mutational profile of the other subject into a trained similarity model, the trained similarity model having been trained to generate a similarity weight representing a predicted degree to which the mutational profile of the particular subject is similar to the other mutational profile of the other subject;
determining, based on the similarity weights outputted by the trained similarity model, a predicted treatment outcome of performing the line of therapy on the particular subject, wherein:
upon determining that at least one of the similarity weights outputted by the similarity model is within a threshold, identifying one of the other subjects based on the determination and assigning the treatment outcome of the identified other subject as the predicted treatment outcome for the particular subject; and/or
upon determining that none of the similarity weights outputted by the similarity model are within the threshold, identifying another set of subjects having been diagnosed with a different type of cancer than the particular subject to search for a mutational profile that is similar to the mutational profile of the particular subject.

15. A computer-program product tangibly embodied in a non-transitory machine-readable storage medium, including instructions configured to cause one or more data processors to perform the following operations:
identifying a particular subject having been diagnosed with a type of cancer, wherein a line of therapy is proposed to be performed on the particular subject;
retrieving a genomic data set corresponding to the particular subject, the genomic data set including a mutational profile indicating one or more molecular characteristics of the particular subject;
identifying a set of other subjects having been diagnosed with the same type of cancer as the subject, and each other subject having undergone the line of therapy and being associated with a treatment outcome;
retrieving another genomic data set for each other subject of the set of other subjects, the other genomic data set including another mutational profile;
inputting, for each other subject of the set of other subjects, the mutational profile of the particular subject and the other mutational profile of the other subject into a trained similarity model, the trained similarity model having been trained to generate a similarity weight representing a predicted degree to which the mutational profile of the particular subject is similar to the other mutational profile of the other subject;
determining, based on the similarity weights outputted by the trained similarity model, a predicted treatment outcome of performing the line of therapy on the particular subject, wherein:
upon determining that at least one of the similarity weights outputted by the similarity model is within a threshold, identifying one of the other subjects based on the determination and assigning the treatment outcome of the identified other subject as the predicted treatment outcome for the particular subject; and/or
upon determining that none of the similarity weights outputted by the similarity model are within the threshold, identifying another set of subjects having been diagnosed with a different type of cancer than the particular subject to search for a mutational profile that is similar to the mutational profile of the particular subject.

16. The computer-program product, as recited in claim 15, wherein the operations further comprise:
retrieving yet another mutational profile for each other subject of the other set of other subjects, each other subject of the other set having a different type of cancer than the particular subject;
inputting, for each other subject of the other set of other subjects, the mutational profile of the particular subject and the other mutational profile of the other subject of the other set into the trained similarity model;
determining, based on the similarity weights outputted by the trained similarity model, that at least one of the similarity weights outputted by the similarity model is within the threshold; and
identifying one of the other subjects of the other set based on the determination and assigning the treatment outcome of the identified other subject of the other set as the predicted treatment outcome for the particular subject; and/or
wherein the mutational profile includes a mutational profile associated with the particular subject, wherein the mutation order represents a series of multiple genetic mutations that mutated at different times.

17. The computer-program product, as recited in claim 15, wherein the operations further comprise:
performing a clustering operation on a set of other subject records, the clustering operation being based on one or more outcomes of the line of therapy and forming one or more clusters.

18. The computer-program product, as recited in claim 15, wherein the similarity model is trained using a training data set, wherein the training data set includes pairs of mutational profiles labeled as being similar or not similar.

19. The computer-program product, as recited in claim 15, wherein the predicted treatment outcome includes one or more subject-specific side effects or a progression-free survival specific to characteristics of the particular subject.

20. The computer-program product, as recited in claim 15, wherein contextual information associated with the particular subject includes the genomic profile associated with the subject.
